(19) United States
(12) Patent Application Publication
(10) Pub. No.: US 2026/0102502 A1
(43) Pub. Date: Apr. 16, 2026

(54) CANNABINOID DENDRIMER COMPOSITIONS FOR TARGETED DELIVERY

(71) Applicant: The Johns Hopkins University, Baltimore, MD (US)

(72) Inventors: Kannan Rangaramanujam, Highland, MD (US); Sujatha Kannan, Highland, MD (US); Kunal Parikh, Reynoldsburg, OH (US); Anjali Sharma, Baltimore, MD (US); Wathsala Liyanage, Ellicott City, MD (US)

(21) Appl. No.: 19/106,535

(22) PCT Filed: Aug. 25, 2023
(86) PCT No.: PCT/US2023/072955

§ 371 Date: Feb. 25, 2025

Related U.S. Application Data
(60) Provisional application No. 63/401,464, filed on Aug. 26, 2022.

(51) Int. Cl. A61K 47/59; A61K 47/61; A61P 25/02

(52) U.S. Cl. CPC A61K 47/595; A61K 47/61; A61P 25/02

(57) ABSTRACT

Dendrimer formulations including one or more cannabinoids and/or cannabinoid derivatives, and methods of use thereof are used for targeted delivery, especially to neurons and activated microglia, or other cells expressing CB1 or CB2 receptors thereon. The cannabinoid-dendrimer conjugate can be administered to patients in need of treatment for a variety of conditions including central and peripheral nerve disorders, chronic pain, neuropathic pain, neurodegeneration (aging, dementia, Alzheimer's), traumatic brain injury, seizures, Huntington's disease and multiple sclerosis, epilepsy, inflammatory disorders, and pain.

### FIELD OF THE INVENTION

This invention is generally in the field of cannabinoid drug formulations, specifically dendrimer-cannabinoid conjugates for selective delivery to the cannabinoid family of receptors in the CNS and other sites of disease.

### CROSS-REFERENCE TO RELATED APPLICATIONS

This application claims benefit of U.S. Provisional Application No. 63/401,464 filed on Aug. 26, 2022 entitled “CANNABINOID DENDRIMER COMPOSITIONS FOR TARGETED DELIVERY” by The Johns Hopkins University, listing inventors Kannan Rangaramanujam, Sujatha Kannan, Kunal Parikh, and Anjali Sharma, hereby incorporated herein by reference in its entirety.

### STATEMENT REGARDING FEDERALLY SPONSORED RESEARCH

None.

### BACKGROUND OF THE INVENTION

Cannabinoids are beneficial for a range of clinical conditions, including pain, inflammation, epilepsy, sleep disorders, the symptoms of multiple sclerosis, anorexia, schizophrenia and other conditions. The transformation of cannabinoids from herbal preparations into highly regulated prescription drugs is therefore progressing rapidly. Drugs, such as cannabinoids, that are metabolized by liver and gut enzymes (first-pass hepatic metabolism), have specific pharmacokinetic requirements, demonstrate poor gastrointestinal permeability and cause irritation and therefore require alternatives to systemic oral delivery. The low oral bioavailability and highly lipophilic nature of the cannabinoids makes delivery to selected targets difficult. Oils and capsules currently allow for more convenient and accurate dosing than juices or teas from the raw plant but are not well suited for pharmacologic applications.

Cannabinoids are highly lipophilic molecules (log P 6-7) with very low aqueous solubility (2-10 g/mL), that are susceptible to degradation, especially in solution, via the action of light and temperature as well as via auto-oxidation. Formulation plays a crucial role in increasing the solubility and physicochemical stability of the drugs. Commonly used strategies in marketed products include salt formation (i.e., pH adjustment), cosolvency (e.g., ethanol, propylene glycol, PEG400 etc.), micellization (e.g., polysorbate 80, cremophor ELP etc.), (nano)-(micro)-emulsification, complexation (e.g., cyclodextrins), and encapsulation in lipid-based formulations (e.g., liposomes) and nanoparticles. Formulations for the transdermal route, intranasal administration and transmucosal adsorption have been proposed. However, absorption is slow, erratic and variable. Maximal plasma concentrations are usually achieved after 60-120 min, although this can take even longer (up to 6 h) and can be delayed. Furthermore, metabolism produces psychoactive metabolites. Extensive first-pass liver metabolism further reduces the oral bioavailability of THC, while effect duration varies from 8 to 20 h.

The psychotropic effects of cannabis are principally mediated by CB1, which is widely distributed throughout the brain, but mainly in the frontal cortex, basal ganglia and cerebellum. CB1 is also present in several tissues and organs, including adipose tissue, the gastrointestinal tract, the spinal cord, the adrenal and thyroid glands, liver, reproductive organs and immune cells. The presence of CB1 receptors on chondrocytes and osteocytes, as well as evidence for their presence on fibroblast-like synoviocytes, makes CB1 targeting particularly valuable in rheumatic diseases. CB1 activation inhibits adenylate cyclase and reduces cAMP levels and protein kinase A (PKA) activity, resulting in the activation of the A-type potassium channels and decreased cellular potassium levels.

CB2 is principally expressed in immune cells, but can also be found in various other cell types. It is also present in some nervous tissues, such as dorsal root ganglia and microglial cells. CB2 shows 44% amino acid similarity with CB1, and similarly inhibits adenylate cyclase as well as activating mitogen-activated protein kinase. CB2 activation can increase intracellular calcium levels via phospholipase C. While both CB1 and CB2 are coupled to G-proteins, the transduction pathways that they activate can be different, for example, in their interactions with ion channels. A variant of CB2, known as Q63R, is associated with coeliac disease, immune thrombocytopenic purpura and juvenile idiopathic arthritis. Production and release of endocannabinoids are mediated, during inflammatory-joint disease, by the generation of pro inflammatory cytokines such as interferon [IFN]-c, interleukin (IL-12, IL-15, IL-17, IL-18), chemokines, chemical mediators, such as nitric oxide synthetase (NOS)-2, cyclooxygenase-2 (COX-2), matrix metalloproteinases (MMPs) and various other arachidonic acid metabolic by-products.

There is no effective treatment with which to prevent or reverse neuropathic pain, thus current treatment is only directed at reducing symptoms. The treatment of chronic pain is still an unmet clinical need, where adequate pain relief is obtained using drugs with adverse effects on central nervous system side. The quality of life of neuropathic pain patients is often aggravated by comorbidities such as sleep disorders, depression and anxiety compromise. Preclinical studies have shown that cannabinoid receptor agonists block pain in various acute and chronic pain models and that inflammation is attenuated. Both CB1 and CB2 receptor agonists demonstrate anti-nociceptive activity, whether used singly or in combination, with CB2 activity believed to affect microglial cells and thereby reduce neuro-inflammatory mechanisms. The CB2 receptor is thought to be particularly important in central neuronal pain circuits, as agonist activity induces dopamine release in mid-brain areas, contributing to descending pain control.

THC is the primary psychoactive component of cannabis and works primarily as a partial agonist of CB1 (Ki=53 nM) and CB2 (Ki=40 nM) receptors and has well-known effects on pain, appetite enhancement, digestion, emotions and processes that are mediated through the endocannabinoid system Production and release of endocannabinoids are mediated, during inflammatory-joint disease, by the generation of pro inflammatory cytokines (interferon [IFN]-c, interleukin (IL-12, IL-15, IL-17, IL-18), chemokines, chemical mediators, such as nitric oxide synthetase (NOS)-2, cyclooxygenase-2 (COX-2), matrix metalloproteinases (MMPs) and various other arachidonic acid metabolic by-products. Adverse psychoactive events can be caused by THC, depending on dose and previous patient tolerance. By contrast CBD, which is the major non-psychoactive phytocannabinoid component of C. sativa, has little affinity for these receptors, (Ki for human CB1 and CB2 of 1.5 and 0.37 μM, respectively), and acts as a partial antagonist CB1 and as a weak inverse CB2 agonist.

It is an object of the present invention to provide formulations with improved bioavailability, pharmacokinetics, and increased selectivity of delivery, and reduced side effects.

### SUMMARY OF THE INVENTION

Cannabinoid-dendrimer conjugates have been developed. The compositions may include one or more endogenous cannabinoids or endocannabinoids. Endocannabinoids are lipid-signaling molecules that are produced from within the body that activate cannabinoid receptors and mimic the activity of Δ9-tetrahydrocannabinol, the main psychotropic constituent of cannabis. Exemplary endocannabinoids and endocannabinoid analogs that may be included in the compositions include, but are not limited to, N-arachidonoylethanolamide (anandamide), 2-arachidonoylglycerol (2-AG), 2-Arachidonyl glyceryl ether (noladin ether), N-Arachidonoyl dopamine (NADA), Virodhamine (OAE), Lysophosphatidylinositol (LPI), 7,10,13,16-docosatetraenoylethanolamide and homo-γ-linolenoylethanolamine. Phytocannabinoids or phytocides are a structurally diverse class of naturally occurring chemical constituents derived from plants. In some forms, the phytocannabinoids or phytocannabinoid derivatives used in the compositions may be derived from various sources, including but not limited to hemp, cannabis, Echinacea, Acmella oleracea, Helichrysum umbraculigerum, Radula marginata, kava, black truffle, Syzygium aromaticum (cloves), Rosmarinus oficinalis, basil, oregano, black pepper, lavender, true cinnamon, malabathrum. cananga odorata, copaifera spp., and hops. Examples include THC, Δ9-tetrahydrocannabinol; Δ9-THCV, Δ9-tetrahydrocannabivarin; CBN, cannabinol; CBDV, cannabidivarin; CBG, cannabigerol; CBC, and cannabichromene.

The compositions may contain one or more phytocannabinoids or phytocannabinoid derivatives belonging to one or more of the following subclasses: cannabidiols (CBDs), tetrahydrocannabinols (THCs), cannabigerols (CBGs), cannabinols (CBNs), cannabichromenes (CBCs), cannabielsoins (CBEs), cannabicyclols (CBLs), cannabifurans (CBFs), cannabitran (CBTs), Cannabinodivarins (CBVs), cannabiripsol (CBR), isocannabinoids and/or derivatives thereof.

The dendrimers are preferably glucose dendrimers, hydroxyl-terminated PAMAM dendrimers, or sugar-modified dendrimers, most preferably glucose dendrimers. Preferred glucose dendrimers include G1, G2, and G3 glucose dendrimers, while preferred PAMAM dendrimers include G3, G4, G5, and G6 hydroxyl-terminated PAMAM dendrimers. The density and loading of cannabinoid on the dendrimer, the means of attachment of the cannabinoids to the dendrimer, the size and chemical composition of the dendrimer as well as the size and composition of linker to the cannabinoid, if any, govern the rate, selectivity, and activity of the cannabinoid to the site of delivery where the cannabinoid binds to receptor. In a preferred embodiment, a cleavable linker binds the cannabinoid to the dendrimer. In the most preferred embodiment, the cleavable linker is an ester. In some forms, the linker contain a triazole moiety.

Route of administration, typically oral, intranasal or applied to other mucosal surface, affects the rate and dosage of the dendrimer conjugate to the brain. The dendrimer increases the brain uptake, solubility, target engagement, PK, and helps confine the drug to the right compartment. In some embodiments, the dendrimer is used to retain the cannabinoid in the peripheral circulation rather than the central circulation.

The cannabinoid-dendrimer conjugate can be administered to patients in need of treatment for a variety of conditions including neurodegeneration (aging, dementia, Alzheimer's), psychosis, acute, chronic and neuropathic pain, cancer-related pain, traumatic brain injury, seizures, Huntington's disease and multiple sclerosis, epilepsy, inflammatory disorders, and control of nausea in chemotherapy.

### BRIEF DESCRIPTION OF THE DRAWINGS

FIGS. 1A and 1B are schematics of how the differential cannabinoid (CB) receptor signaling modalities can impact neuromodulation in health and disease in specific ways. FIG. 1A shows the key enzymes such as diacylglycerol lipase (dglα) and phospholipase d (PLD) that produce the endogenous ligands arachidonylethanolamine (AEA) and 2-arachidonvlglvcerol (2-AG). These activate the cannabinoid 1 receptor (CB₁) receptor in the central nervous system (CNS). The result can include modulation of adenylate cyclase activity to inhibit cAMP accumulation, voltage-gated calcium channels (VGCC), K+ channels and neurotransmitter release in presynaptic excitatory and inhibitory synapses. FIG. 1B shows that, following activation of the CB₁ receptor by ligand binding, signaling via G protein and/or 0-arrestin may occur at the plasma membrane, in endocytic pits or in endosomes after internalization of the receptor. G proteins usually bind the unphosphorylated receptor while β-arrestin binds the receptor phosphorylated by G protein receptor kinases. FIG. 1C is a schematic of key receptors and cells in the brain where dendrimer-cannabinoid conjugates could exert selective action.

FIG. 2A is a schematic of a stepwise synthetic route for producing an exemplary dendrimer-cannabidiol conjugate via click chemistry with an enzyme sensitive ester linkage. FIG. 2B is a schematic of a stepwise synthetic route for conjugating an exemplary cannabinoid, Cannabidiol, to the dendrimer.

FIGS. 3A and 3B are schematics showing molecular structures in a stepwise synthetic route for producing an exemplary dendrimer-cannabidiol conjugate via click chemistry with a non-cleavable ether linkage (FIG. 3A), and a stepwise synthetic route for conjugating an exemplary cannabinoid, Cannabidiol, to the dendrimer (FIG. 3B).

FIGS. 4A and 4B are schematics showing the stepwise synthetic route for producing an exemplary dendrimer-HU-308 conjugate via an amidation reaction with a phosphatase-cleavable phosphodiester linkage (FIG. 4A), and a stepwise synthetic route for conjugating an exemplary cannabinoid, HU-308, to the dendrimer (FIG. 4B).

FIG. 5A is a schematic of a stepwise synthetic route for producing an exemplary dendrimer-HU-308 conjugate via a click reaction with an ester linkage. FIG. 5B is a schematic of a stepwise synthetic route for conjugating an exemplary cannabinoid, HU-308, to the dendrimer. FIG. 5C is a schematic of the conjugation of the HU-308 to a PAMAM-G4-OH dendrimer.

FIGS. 6A and 6B are schematics showing molecular structures in a stepwise synthetic route for producing an exemplary dendrimer-HU-308 conjugate via click reaction with glutathione sensitive disulfide linkage (FIG. 6A), and a stepwise synthetic route for conjugating an exemplary cannabinoid, HU-308, to the dendrimer to produce D-HU-308 conjugate with a disulfide linker (FIG. 61B).

FIG. 7A is a schematic of a stepwise synthetic route for producing an exemplary dendrimer-tetrahydrocannabinol (D-THC) conjugate via a click reaction with an esterase sensitive ester linkage. FIG. 7B is a schematic of a stepwise synthetic route for conjugating an exemplary cannabinoid, THC, to the dendrimer to produce a D-THC conjugate.

FIGS. 8A and 8B are schematics showing molecular structures in a stepwise synthetic route for producing an exemplary dendrimer-THC conjugate via click reaction with a triglycyl peptide linker suitable for lysosomal release (FIG. 8A), and a stepwise synthetic route for conjugating an exemplary cannabinoid, THC, to the dendrimer to produce a D-THC conjugate with a triglycyl peptide linker (FIG. 8B).

FIG. 9A is a schematic of a stepwise synthetic route for producing an exemplary dendrimer-anandamide (D-AEA) conjugate via a click reaction.

FIG. 9B is a schematic of a stepwise synthetic route for conjugating an exemplary cannabinoid, anandamide, to the dendrimer to produce a D-AEA conjugate.

FIGS. 10A and 10B are schematics of a stepwise synthetic route for producing an exemplary dendrimer-AEA, with a non-cleavable ether linkage (FIG. 10A), and a stepwise synthetic route for conjugating an exemplary cannabinoid, AEA, to the dendrimer to produce a D-AEA conjugate with a non-cleavable ether linker (FIG. 10B).

FIG. 11A is a schematic of a stepwise synthetic route for producing an exemplary dendrimer-2-arachidonoylglycerol (D-2-AG) conjugate via a click reaction. FIG. 11B is a schematic of a stepwise synthetic route for conjugating an exemplary cannabinoid, D-2-AG, to the dendrimer to produce a D-2-AG conjugate.

FIG. 12 is a table of the structures of Hu308 and cannabidiol and conjugatable analogs thereof. FIGS. 13A-13B show the synthesis and structures shown in FIG. 12.

FIG. 13C show the binding and release of the compounds.

FIG. 14 is a graph of % efficiency of binding in the 5-HT1A human serotonin GPCR cell based agonist cAMP assay versus log of tryptamine in μM.

### DETAILED DESCRIPTION OF THE INVENTION

### I. Definitions

“Cannabinoid”, as used herein, refers to a lipid-based compound that has direct or indirect activity at a cannabinoid receptor. “Endocannabinoids” are endogenous cannabinoids generated naturally inside the body and are found in humans and other animals. “Exogenous cannabinoids” include phytocannabinoids which are cannabinoids derived from the Cannabis plants and related synthetic derivatives thereof.

The terms “active agent” or “biologically active agent” are used interchangeably to refer to a chemical or biological compound that induces a desired pharmacological and/or physiological effect, which may be prophylactic, therapeutic, or diagnostic. These may be a nucleic acid, a nucleic acid analog, a small molecule having a molecular weight less than 2 kD, more typically less than 1 kD, a peptidomimetic, a protein or peptide, carbohydrate or sugar, lipid, or a combination thereof. The terms also encompass pharmaceutically acceptable, pharmacologically active derivatives of agents, including, but not limited to, salts, esters, amides, prodrugs, active metabolites, and analogs. An analog is a chemically modified active compound derived from the parent. The term “therapeutic agent” refers to an agent that can be administered to treat one or more symptoms of a disease or disorder. The term “diagnostic agent” generally refers to an agent that can be administered to reveal, pinpoint, and define the localization of a pathological process. The diagnostic agents can label target cells that allow subsequent detection or imaging of these labeled target cells.

The term “therapeutically effective amount” refers to an amount of the therapeutic agent that, when incorporated into and/or onto dendrimers, produces some desired effect at a reasonable benefit/risk ratio applicable to any medical treatment. The effective amount may vary depending on such factors as the disease or condition being treated, the particular targeted construct being administered, the size of the subject, or the severity of the disease or condition. One of ordinary skill in the art may empirically determine the effective amount of a particular compound without necessitating undue experimentation. In some embodiments, the term “effective amount” refers to an amount of a therapeutic agent or prophylactic agent to reduce or diminish the symptoms of one or more diseases.

The terms “inhibit” or “reduce” in the context of inhibition, mean to reduce, or decrease in activity and quantity. This can be a complete inhibition or reduction in activity or quantity, or a partial inhibition or reduction. Inhibition or reduction can be compared to a control or to a standard level. Inhibition can be 5, 10, 25, 50, 75, 80, 85, 90, 95, 99, or 100%. For example, dendrimer compositions including one or more inhibitors may inhibit or reduce the activity and/or quantity of diseased neurons by about 10%, 20%, 30%, 40%, 50%, 75%, 85%, 90%, 95%, or 99% from the activity and/or quantity of the same cells in equivalent tissues of subjects that did not receive, or were not treated with the dendrimer compositions. In some embodiments, the inhibition and reduction are compared at levels of mRNAs, proteins, cells, tissues, and organs. For example, an inhibition and reduction in the rate of neural loss, in the rate of decrease of brain weight, or in the rate of decrease of hippocampal volume, as compared to an untreated control subject.

The term “treating” or “preventing” mean to ameliorate, reduce or otherwise stop a disease, disorder or condition from occurring or progressing in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating, or palliating the disease state, and remission or improved prognosis. For example, an individual is successfully “treated” if one or more symptoms associated with depression are mitigated or eliminated, including, but are not limited to, reducing the level of anxiety, agitation, or restlessness, improving feelings of sadness, tearfulness, emptiness or hopelessness, increasing the quality of life of those suffering from the disease, decreasing the dose of other medications required to treat the disease, delaying the progression of the disease.

The phrase “pharmaceutically acceptable” or “biocompatible” refers to compositions, polymers, and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions, or vehicles, such as a liquid or solid filler, diluent, solvent, or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

The term “biodegradable” generally refers to a material that will degrade or erode under physiologic conditions to smaller units or chemical species that are capable of being metabolized, eliminated, or excreted in vivo. The degradation time is a function of composition and morphology.

The term “dendrimer” includes, but is not limited to, a molecular architecture with an interior core, interior layers, or “generations” of repeating units regularly attached to this initiator core, and an exterior surface of terminal groups attached to the outermost generation.

The term “functionalize” means to modify a compound or molecule in a manner that results in the attachment of a functional group or moiety. For example, a molecule may be functionalized by the introduction of a molecule that makes the molecule a strong nucleophile or strong electrophile.

The term “targeting moiety” refers to a moiety that localizes to or away from a specific location. The moiety may be, for example, a protein, nucleic acid, nucleic acid analog, carbohydrate, or small molecule. The location may be a tissue, a particular cell type, a subcellular compartment, or a molecule such as a receptor.

The term “prolonged residence time” refers to an increase in the time required for an agent to be cleared from a patient's body, or organ or tissue of that patient. In certain embodiments, “prolonged residence time” refers to an agent that is cleared with a half-life that is 10%, 20%, 50% or 75% longer than a standard of comparison such as a comparable agent without conjugation to a delivery vehicle such as a dendrimer. In certain embodiments, “prolonged residence time” refers to an agent that is cleared with a half-life of 2, 5, 10, 20, 50, 100, 200, 500, 1000, 2000, 5000, or 10000 times longer than a standard of comparison such as a comparable agent without a dendrimer that specifically target specific cell types.

The terms “incorporated” and “encapsulated” refer to incorporating, formulating, or otherwise including an agent into and/or onto a composition that allows for release, such as sustained release, of such agent in the desired application. The agent or other material can be incorporated into a dendrimer, by binding to one or more surface functional groups of such dendrimer (by covalent, ionic, or other binding interaction), by physical admixture, by enveloping the agent within the dendritic structure, and/or by encapsulating the agent inside the dendritic structure.

As used herein, central nervous system (“CNS”) includes the brain and spinal cord. As used herein, peripheral nervous system (“PNS”) refers to the nerves other than in the brain and spinal cord.

“Hydroxyl-terminated,” as relates to dendrimers, refers to dendrimers that have a hydroxyl group on their surface. These hydroxyl groups are not attached to the termini of the dendrimers via a sugar moiety (such as a saccharide moiety).

“Sugar-terminated,” as relates to dendrimers, refers to dendrimers that contain a sugar moiety (such as a saccharide moiety) on their surface and not in their core.

“Sugar-based,” as relates to dendrimers, refers to dendrimers that contain a sugar moiety (such as a saccharide moiety) in their core, or their core and on their surface.

“Analog” as relates to a given compound, refers to another compound that is structurally similar, functionally similar, or both, to the specified compound. Structural similarity can be determined using any criterion known in the art, such as the Tanimoto coefficient that provides a quantitative measure of similarity between two compounds based on their molecular descriptors. Preferably, the molecular descriptors are 2D properties such as fingerprints, topological indices, and maximum common substructures, or 3D properties such as overall shape, and molecular fields. Tanimoto coefficients range between zero and one, inclusive, for dissimilar and identical pairs of molecules, respectively. A compound can be considered an analog of a specified compound, if it has a Tanimoto coefficient with the specified compound between 0.5 and 1.0, inclusive, preferably between 0.7 and 1.0, inclusive, most preferably between 0.85 and 1.0, inclusive. A compound is functionally similar to a specified compound, if it induces the same pharmacological effect, physiological effect, or both, as the specified compound. “Analog” can also refer to a modification including, but not limited to, hydrolysis, reduction, or oxidation products, of the compounds. Hydrolysis, reduction, and oxidation reactions are known in the art.

### II. Compositions

Dendrimer-active agent conjugates suitable for delivering one or more cannabinoids and/or cannabinoid derivatives to one or more target cells expressing receptors therefor, such as neural and/or glial cells, have been developed. Generally, the cannabinoids and/or cannabinoid derivatives bind to a receptor on the surface of the target cells and/or a receptor inside the target cells. Exemplary target cells include, but not limited to, brain cells such as microglia, astrocytes, and/or neurons, for example, those within the site of pathology in the brain or the CNS; and/or cells in the peripheral nervous system, such as peripheral neurons and glia. The microglia and/or astrocytes to which the cannabinoids and/or cannabinoid derivatives are delivered may be activated or inactive microglia and/or astrocytes.

The cannabinoids and/or cannabinoid derivatives of the dendrimer-active agent conjugate binds to a target receptor on the surface of the target cell or inside the target cell. In some embodiments, when the cannabinoids and/or cannabinoid derivatives binds to the target receptor, the agent remains conjugated to the dendrimer. In these embodiments, following binding, the agent may be released from the dendrimer or remain conjugated to the dendrimer as an intact dendrimer-active agent conjugate. In some embodiments, the cannabinoids and/or cannabinoid derivatives are released from the dendrimer at close proximity to the target receptor and then binds to the target receptor on the target neural and/or glial cell.

### A. Dendrimers

Dendrimers are three-dimensional, hyperbranched, monodispersed, globular and polyvalent macromolecules including surface end groups (Tomalia, D. A., et al., Biochemical Society Transactions, 35, 61 (2007); and Sharma, A., et al., ACS Macro Letters, 3, 1079 (2014)).

The term “dendrimer” includes, but is not limited to, a molecular architecture with an interior core (“G0”) and layers (or “generations”) of repeating units which are attached to and extend from this interior core, each layer having one or more branching points, and an exterior surface of terminal groups attached to the outermost generation. In some embodiments, dendrimers have regular dendrimeric molecular structures, in other cases they can be hyperbranched structures with irregular branch lengths.

Generally, the dendrimers have a diameter between about 1 nm and about 60 nm, more preferably between about 1 nm and about 50 nm, between about 1 nm and about 40 nm, between about 1 nm and about 30 nm, between about 1 nm and about 20 nm, between about 1 nm and about 10 nm, or between about 1 nm and about 5 nm. In some embodiments, the diameter is between about 1 nm to about 2 nm. The preferred size of the dendrimers for crossing the blood brain barrier (“BBB”) is less than 5 nm, whereas those for not crossing the BBB and staying in the peripheral circulation are greater than 5 nm. In some embodiments, the dendrimers have a diameter effective to penetrate the BBB and to be retained close to or within target neural and/or glial cells for delivery of the agents conjugated thereto. In some embodiments, the dendrimers have a diameter effective to penetrate a BBB and to be internalized into target neural and/or glial cells for delivery of the agents conjugated thereto, such as for example, neurons, oligodendrocytes, astrocytes, microglial, and neuroglial support cells. In some embodiments, the dendrimers have a diameter effective to penetrate a barrier interface, such as a blood nerve barrier (“BNB”), and to be intemalized into neural and glial cells of the peripheral nervous system for delivery of the agents conjugated thereto such as for example, neurons, Schwann cells, satellite cells, and neuroglial support cells. In some embodiments, the dendrimers have a diameter effective to be retained in the peripheral circulation for delivery of the agents conjugated thereto to target cells of the peripheral nervous system.

In some embodiments, dendrimers have a molecular weight between about 500 Daltons and about 100,000 Daltons inclusive, between about 500 Daltons and about 50,000 Daltons inclusive, or between about 1,000 Daltons and about 20,000 Daltons inclusive. Dendrimer sizes <30,000 Da are preferred for transport across the BBB, and sizes of >50,000 Da are preferred for confinement to the periphery.

In some embodiments, the dendrimers have a hypercore (e.g., dipentaerythritol) and one or more monosaccharide branching units. In some embodiments, the monosaccharide branching units are conjugated to the core or the prior layer of monomers via linkers such as polyethylene glycol chains. In preferred embodiments, the hypercore is dipentaerythritol and the monosaccharide branching unit is glucose-based branching unit such as shown in Structures II-IV. In the most preferred embodiment, the dendrimers are made entirely of glucose building blocks. PAMAM dendrimers modified by sugar may also work, but dendrimers made of sugars, especially glucose, are most preferred. Particularly preferred glucose dendrimers are G1 to G3 glucose dendrimers, such as G1, G2, and/or G3 glucose dendrimers.

Suitable dendrimers scaffolds for use in the conjugates include, but are not limited to, poly(amidoamine), also known as PAMAM, or STARBURST™ dendrimers; polypropylamine (POPAM), polyethylenimine, polylysine, polyester, iptycene, aliphatic poly(ether), aromatic polyether dendrimers, dendrimer of a sugar (e.g., glucose, galactose, mannose, fructose, etc.), and copolymers thereof, such as a copolymer of a sugar and an alkylene glycol (e.g., a dendrimer formed by glucose and ethylene glycol building blocks). The dendrimers can have a plurality of surface functional groups, such as carboxylic, amine, hydroxyl, and/or acetamide. The terms “surface functional groups” and “terminal groups” are used interchangeably herein. In some embodiments, the dendrimers have surface hydroxyl groups. In some embodiments, one or more of these surface functional groups are further modified with other molecules, such as further modified with a sugar (e.g., glucose, galactose, mannose, fructose, etc.) and/or a polyalkylene glycol, for example, polyethylene glycol, and thus have sugar molecules and/or polyalkylene glycols as terminal moieties/molecules. Preferred PAMAM dendrimers include hydroxyl-terminated PAMAM dendrimers, particularly G3 to G6 hydroxyl-terminated PAMAM dendrimers, such as G3, G4, G5, and G6 hydroxyl-terminated PAMAM dendrimers. Dendrimers can be any generation including, but not limited to, generation 1, generation 2, generation 3, generation 4, generation 5, generation 6, generation 7, generation 8, generation 9, or generation 10. In some embodiments, the dendrimers are PAMAM dendrimers used as a platform and modified with functional groups for increased number of surface hydroxyl groups.

In some embodiments, the dendrimer-active agent conjugates can be confined to the peripheral circulation and specifically target a particular tissue region and/or cell type, such as peripheral neural and macrophage cells, by using higher generation dendrimer (such as generation 4, 5, or 6 PAMAM dendrimer, generation 2, 3, or higher glucose-based dendrimers). Additionally, or alternatively, the dendrimer-active agent conjugates can be confined to the peripheral circulation by appropriate functionalization of the dendrimer (such as PEGylation).

In some embodiments, the dendrimers can specifically target a particular tissue region and/or cell type of the central nervous system (CNS), the peripheral nervous system (PNS), and/or the eye, such as neurons and glia of the CNS, and/or PNS, by using dendrimers of a certain generation, such as PAMAM dendrimers and/or glucose dendrimers of generation 2 (G2), G3, G4, and G5. In the most preferred embodiment, the dendrimers are made entirely of glucose building blocks. PAMAM dendrimers modified by sugar may also work, but dendrimers made of sugars, especially glucose, are most preferred.

### Monosaccharide-Based Dendrimers

In some embodiments, the branching units include monosaccharides. In some embodiments, the monosaccharide branching units are conjugated to the core or the prior layer of monomers via linkers such as polyethylene glycol chains. In some embodiments, the monosaccharide branching units are glucose-based branching units. In some embodiments, the branching units can include PEG and/or alkyl chain linkers between different dendrimer generations. For example, the glucose layers are connected via PEG linkers and triazole rings. In some embodiments, the branching units are the same for each generation of dendrimers generated from the core. Therefore, for example, the branching units are glucose-based branching units for generating generation 1 dendrimers, for generating generation 2 dendrimers, and for generating generation 3 dendrimers.

In some embodiments, the dendrimers have a hypercore (e.g., dipentaerythritol) and one or more monosaccharide branching units. In some embodiments, the hypercore is dipentaerythritol and the monosaccharide branching unit is glucose-based branching unit. In further embodiments, spacer molecules can also be alkyl (CH₂)ₙ-hydrocarbon-like units.

In some embodiments, dendrimers synthesized using glucose building blocks, with a surface made predominantly of glucose moieties, allow specific targeting in cells including injured neurons, ganglion cells, and other neuronal cells in the brain, the eye, and/or in peripheral nervous system. In some embodiments, the glucose-based dendrimer selectively targets or is enriched inside target neural and/or glial cells. In some embodiments, the glucose-based dendrimer selectively targets or enriched on the surface of target neural and/or glial cells. In some embodiments, the glucose-based dendrimer selectively targets or enriched inside target neuronal cells and on the surface of the target neural and/or glial cells. In some embodiments, the glucose-based dendrimer selectively targets or enriched inside and/or on the surface of injured, diseased, and/or hyperactive neurons and/or glial cells.

In some cases, the dendrimers include an effective number of sugar molecules and terminal groups, for example, glucose and/or hydroxyl groups, for targeting to one or more neurons and/or glia of the CNS, PNS, and/or the eye. The terminal hydroxyl groups of these dendrimers may be part of terminal glucose molecules or extra hydroxyl groups that are not part of the glucose molecules, or a combination thereof. In some embodiments, all the terminal hydroxyl groups are part of the terminal glucose molecules. In some embodiments, the number of sugar molecules on the termination of dendrimer is determined by the generation number.

In some embodiments, dendrimers are made of glucose and oligoethylene glycol building blocks. Exemplary glucose dendrimers are shown in Structures V and VII.

Some exemplary glucose dendrimers include a generation 1 glucose dendrimer having 24 hydroxyl (—OH) end groups, a generation 2 glucose dendrimer having 96 hydroxyl (—OH) end groups, a generation 3 glucose dendrimer having 396 hydroxyl (—OH) end groups, and generation 4 glucose dendrimer having 1584 hydroxyl (—OH) end groups. For example, the glucose dendrimer is a generation 2 glucose-based dendrimer that has 24 glucose molecules at the periphery and 6 embedded glucose molecules in the backbone held together by PEG segments.

Dendrimer compositions that can selectively accumulate inside neurons, particularly in the nucleus of injured and/or hyperactive neurons, referred to as “glucose dendrimers” have been developed. These dendrimers can also accumulate at a high level inside activated microglia. However, compared to hydroxyl dendrimers which primarily accumulate in microglia, these dendrimers primarily go to neurons. Glucose dendrimer are described in U.S. Ser. No. 63/327,610 “Dendrimer Compositions for Targeted Delivery of Therapeutics to Neurons” by The Johns Hopkins University, inventors Kannan Rangaramanujam, Rishi Sharma, Anjali Sharma, Sujatha Kannan, Nimath Sah, Mira Sachdeva, and Siva P. Kambhampati filed Apr. 5, 2022.

Glucose dendrimers include (a) a central core, (b) one or more branching units, wherein the branching units are monosaccharide glucose-based branching units, optionally with a linker conjugated thereto; and optionally (c) one or more therapeutic, prophylactic and/or diagnostic agents. Generally, the one or more branching units are conjugated to the central core, and the surface groups of the dendrimer are monosaccharide glucose molecules. In some embodiments, the central core is dipentaerythritol, or a hexa-propargylated derivative thereof. In some embodiments, the branching unit is conjugated to the central core via a linker such as a hydrocarbon or an oligoethylene glycol chain. In a preferred embodiment, the branching units are β-D-Glucopyranoside tetraethylene glycol azide having the following structure,

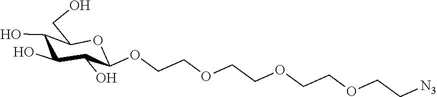

or peracetylated derivatives thereof

In some embodiments, the glucose dendrimer is a generation 1, generation 2, generation 3, generation 4, generation 5, or generation 6 dendrimer. In one embodiment, the dendrimer is a generation 1 dendrimer having the following structure:

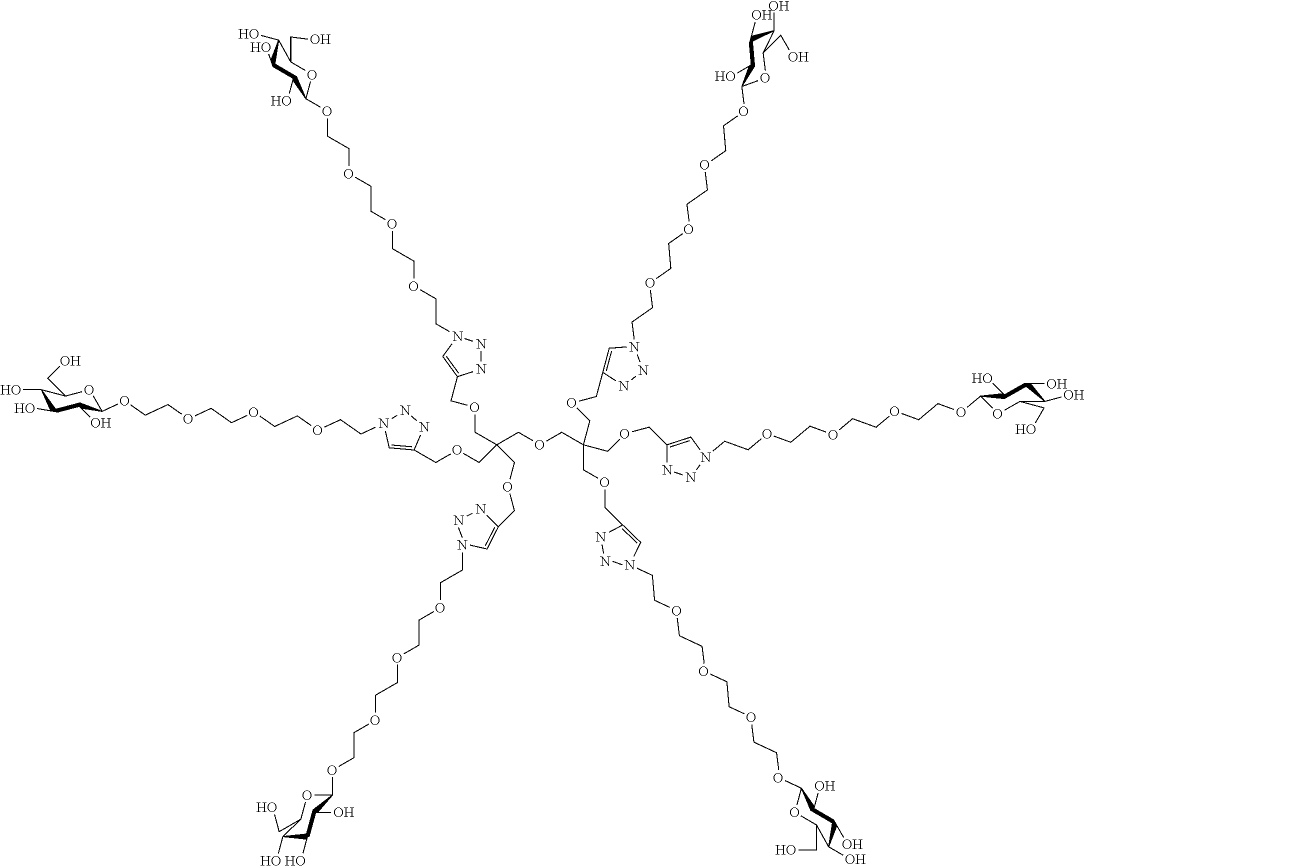

In a preferred embodiment, the dendrimer is a generation 2 dendrimer having the following structure:

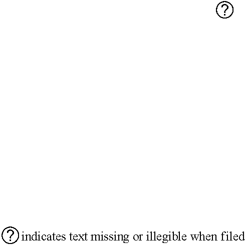

In some embodiments, the one or more therapeutic agents, prophylactic agents, and/or diagnostic agents are encapsulated, associated, and/or conjugated in the dendrimer, at a concentration of between about 0.01% to about 30%, preferably about 1% to about 20%, more preferably about 5% to about 20% by weight. In some embodiments, the dendrimer is conjugated to a small molecule, an antibody or antigen-binding fragment thereof, a nucleic acid, or a polypeptide. In some embodiments, the therapeutic agents conjugated to the dendrimer are anti-inflammatory agents, antioxidant agents, or immune-modulating agents. In other embodiments, the dendrimers are conjugated to one or more diagnostic agents such as fluorescent dyes, near infra-red dyes, SPECT imaging agents, PET imaging agents, and radioisotopes.

In some embodiments, the dendrimer and the therapeutic, prophylactic, or diagnostic agent(s) are conjugated via one or more linkers or coupling agents such as one or more hydrocarbon or oligoethylene glycol chains. Exemplary linkages are disulfide, ester, ether, thioester, and amide linkages.

### PAMAM Dendrimer

The term “PAMAM dendrimer” refers to poly(amidoamine) dendrimer, which may contain different cores, with amidoamine building blocks, and can have carboxylic, amine, acetamide, and/or hydroxyl terminations of any generation including, but not limited to, generation 1 PAMAM dendrimers, generation 2 PAMAM dendrimers, generation 3 PAMAM dendrimers, generation 4 PAMAM dendrimers, generation 5 PAMAM dendrimers, generation 6 PAMAM dendrimers, generation 7 PAMAM dendrimers, generation 8 PAMAM dendrimers, generation 9 PAMAM dendrimers, or generation 10 PAMAM dendrimers. In some embodiment, the dendrimers are generation (“G”) 4, 5 or 6 dendrimers. In some embodiments, the PAMAM dendrimers have hydroxyl terminations.

Generally, the complete architecture of dendrimers can be distinguished into the inner core moiety followed by radially attached branching units (i.e., generations) which are further decorated with chemical functional groups carrying desired terminal groups at the exterior surface of the dendrimers.

In some embodiments, the dendrimers are in nanoparticle form and are described in detail in U.S. Published Application Nos. US 2011/0034422, US 2012/0003155, and US 2013/0136697. For example, the molecular weight of the dendrimers can be varied to prepare polymeric nanoparticles that form particles having properties, such as drug release rate, optimized for specific applications. In general, conjugation to the dendrimer may further improve safety and efficacy of these agents. For example, dendrimer conjugation may change specific receptor activity and/or modify biodistribution. For example, use of higher generation dendrimers and/or dendrimers with molecular weights greater than 24 kDa can confine these agents to the peripheral nervous system in order to preclude their psychoactive effects.

In some embodiments, different variations of dendrimers may be used as a delivery vehicle to conjugate and deliver one or more active agents, including, but not limited to, dendrons and tectodendrimers. Dendrons are dendritic wedges that comprise one type of functionality at the core (functional groups, f=1) and another at the periphery (f=8, 16, 32, etc., . . . ). Tectodendrimers are generally composed of a central dendrimer with multiple dendrimers attached at its periphery.

### 1. Core

In some embodiments, dendrimers are prepared using methods in which the dendrimer is assembled from a multifunctional core, which is extended outward by a series of reactions. A multifunctional core moiety allows stepwise addition of branching units (i.e., generations) around the core.

Exemplary chemical structures suitable as core moieties include dipentaerythritol, pentaerythritol, 2-(aminomethyl)-2-(hydroxymethyl) propane-1,3-diol, 2-ethyl-2-(hydroxymethyl) propane-1,3-diol, 3,3′,3″,3′″-silanetetrayltetrakis (propane-1-thiol), 3,3-divinylpenta-1,4-diene, 3,3′,3″-nitrilotripropionic acid, 3,3′,3″-nitrilotris(N-(2-aminoethyl)propanamide), 3,3′,3″,3′″-(ethane-1,2-diylbis(azanetriyl)) tetrapropanamide, 3-(carboxymethyl)-3-hydroxypentanedioic acid, 2,2′-((2,2-bis((2-hydroxyethoxy)methyl) propane-1,3-diyl)bis(oxy))bis(ethan-1-ol), tetrakis(3-(trichlorosilyl) propyl)silane, 1-Thioglycerol, 2,2,4,4,6,6-hexachloro-1,3,5,215,415,615-triazatriphosphinine, 3-(hydroxymethyl)-5,5-dimethylhexane-2,4-diol, 4,4′,4″-(ethane-1,1,1-triyl)triphenol, 2,4,6-trichloro-1,3,5-triazine, 5-(hydroxymethyl) benzene-1,2,3-triol, 5-(hydroxymethyl)benzene-1,3-diol, 1,3,5-tris(dimethyl(vinyl)silyl)benzene, Carbosiloxane core, nitrilotrimethanol, ethylene diamine, propane-1,3-diamine, butane-1,4-diamine, 2.2′,2″-nitrilotris(ethan-1-ol), alpha cyclodextrin, beta cyclodextrin, gamma cyclodextrin, Cucurbituril, benzene-1,2,3,4,5,6-hexathiol, monosaccharide, disaccharides, trisaccharides, oligosaccharides, or azide-, alkyne-modified moieties thereof. In some embodiments, the core moiety is chitosan. Thus, azide-modified chitosan, or alkyne-modified chitosan are suitable for conjugating to branching units using click chemistry.

In some embodiments, the core moiety is ethylenediamine, or tetra(ethylene oxide). In some embodiments, the core moiety is dipentaerythritol. Exemplary chemical structures suitable for use as core moieties are shown in Table 1 below.

TABLE 1
Structural representation of various building blocks (cores,
branching units, surface functional groups, monomers) for the synthesis of
dendrimers.

| Building Blocks | Structure |
|---|---|
| Dipentaerythritol | 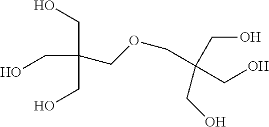 [SMILES: OCC(CO)(CO)COCC(CO)(CO)CO] |
| Pentaerythritol | 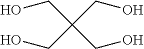 |
| 2-(aminomethyl)-2- (hydroxymethyl) propane-1,3-diol | 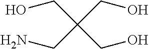 |
| 2-ethyl-2-(hydroxymethyl) propane-1,3-diol | 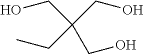 |
| 3,3′,3″,3′′′-silanetetrayltetrakis (propane-1-thiol) | 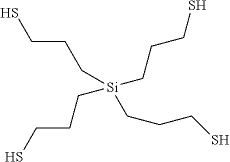 [SMILES: SCCC[Si](CCCS)(CCCS)CCCS] |
| 3,3-divinylpenta-1,4-diene | 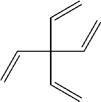 |
| 3,3′,3″-nitrilotripropionic acid | 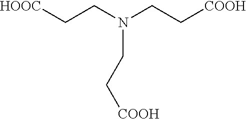 [SMILES: O=C(O)CCN(CCC(=O)O)CCC(=O)O] |
| 3,3′,3″-nitrilotris(N-(2- aminoethyl)propanamide) | 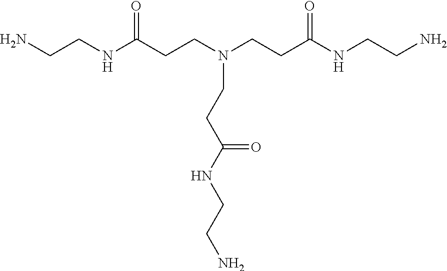 [SMILES: NCCNC(=O)CCN(CCC(=O)NCCN)CCC(=O)NCCN] |
| 3,3′,3″,3′′′-(ethane-1,2- diylbis(azanetriyl)) tetrapropanamide | 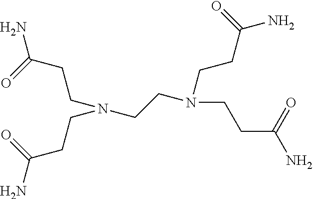 [SMILES: NC(=O)CCN(CCC(N)=O)CCN(CCC(N)=O)CCC(N)=O] |
| 3-(carboxymethyl)-3- hydroxypentanedioic acid | 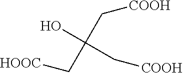 [SMILES: O=C(O)CC(O)(CC(=O)O)CC(=O)O] |
| 2,2′-((2,2-bis((2- hydroxyethoxy)methyl) propane-1,3- diyl)bis(oxy))bis(ethan-1-ol) | 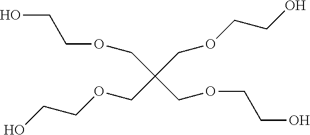 [SMILES: OCCOCC(COCCO)(COCCO)COCCO] |
| tetrakis(3-(trichlorosilyl) propyl)silane | 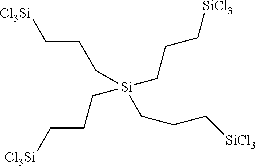 |
| 1-Thioglycerol | 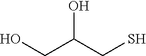 |
| All the sugar based scaffolds including mono, di, tri, or oligomers | 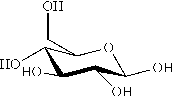 |
| 2,2,4,4,6,6-hexachloro-1,3,5,215, 4l5,6l5-triazatriphosphinine | 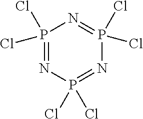 [SMILES: ClP1(Cl)=NP(Cl)(Cl)=NP(Cl)(Cl)=N1] |
| 3-(hydroxymethyl)-5,5- dimethylhexane-2,4-diol | 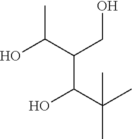 |
| 4,4′,4″-(ethane-1,1,1- triyl)triphenol | 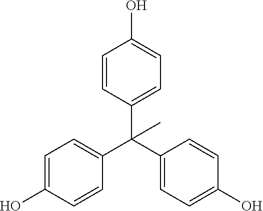 [SMILES: CC(c1ccc(O)cc1)(c1ccc(O)cc1)c1ccc(O)cc1] |
| 2,4,6-trichloro-1,3,5-triazine | 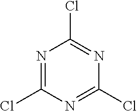 |
| 5-(hydroxymethyl) benzene-1,2,3-triol | 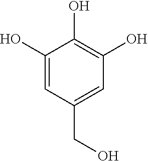 [SMILES: OCc1cc(O)c(O)c(O)c1] |
| 5-(hydroxymethyl)benzene-1,3- diol | 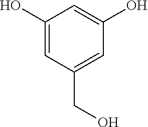 |
| 1,3,5- tris(dimethyl(vinyl)silyl)benzene | 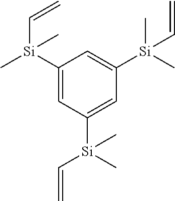 [SMILES: C=C[Si](C)(C)c1cc([Si](C)(C)C=C)cc([Si](C)(C)C=C)c1] |
| Carbosiloxane core | 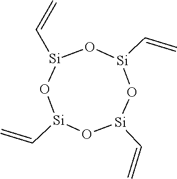 |
| nitrilotrimethanol | 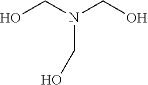 |
| ethylene diamine | 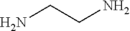 |
| propane-1,3-diamine | 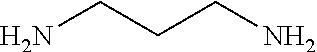 |
| butane-1,4-diamine | 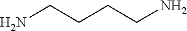 |
| 2,2′,2″-nitrilotris(ethan-1-ol) | 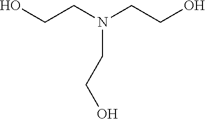 |
| | 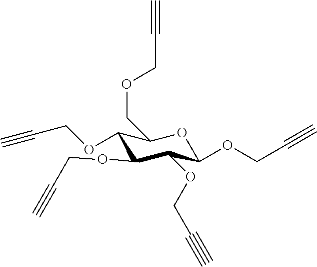 |
| | 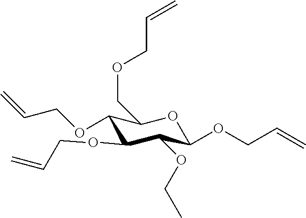 |
| | 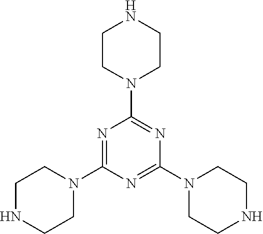 [SMILES: C1CN(c2nc(N3CCNCC3)nc(N3CCNCC3)n2)CCN1] |
| benzene-1,2,3,4,5,6-hexathiol | 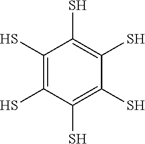 [SMILES: Sc1c(S)c(S)c(S)c(S)c1S] |
| alpha cyclodextrin | 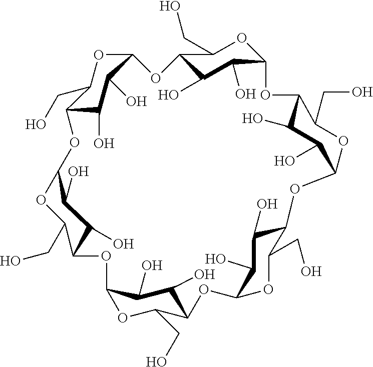 [SMILES: OCC1O[C@@H]2O[C@@H]3C(CO)O[C@H](O[C@@H]4C(CO)O[C@H](O[C@@H]5C(CO)O[C@H](O[C@@H]6C(CO)O[C@H](O[C@@H]7C(CO)O[C@H](O[C@H]1[C@H](O)C2O)C(O)[C@H]7O)C(O)[C@H]6O)C(O)[C@H]5O)C(O)[C@H]4O)C(O)[C@H]3O] |
| beta cyclodextrin | 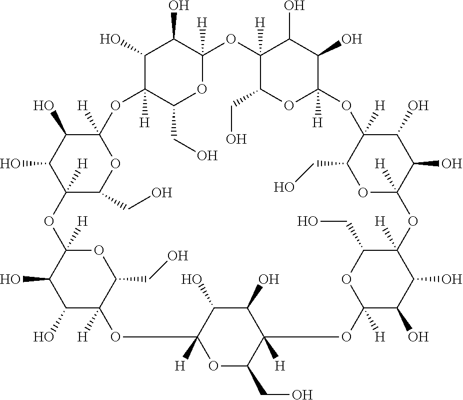 [SMILES: OC[C@H]1O[C@@H]2O[C@H]3C(O)[C@@H](O)[C@@H](O[C@H]4[C@H](O)[C@@H](O)[C@@H](O[C@H]5[C@H](O)[C@@H](O)[C@@H](O[C@H]6[C@H](O)[C@@H](O)[C@@H](O[C@H]7[C@H](O)[C@@H](O)[C@@H](O[C@H]8[C@H](O)[C@@H](O)[C@@H](O[C@H]1[C@H](O)[C@H]2O)O[C@@H]8CO)O[C@@H]7CO)O[C@@H]6CO)O[C@@H]5CO)O[C@@H]4CO)O[C@@H]3CO] |
| gamma cyclodextrin | 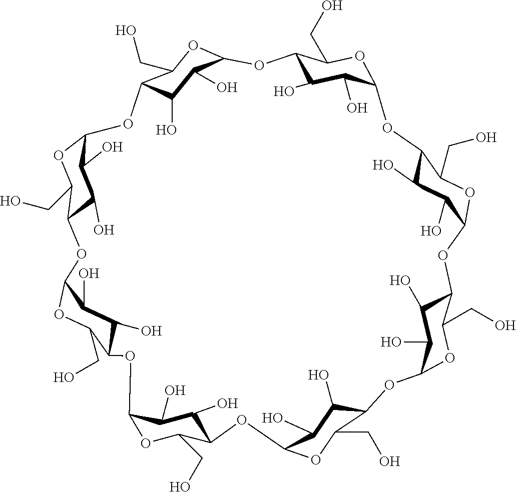 [SMILES: OCC1O[C@@H]2O[C@@H]3C(CO)O[C@H](O[C@@H]4C(CO)O[C@H](O[C@@H]5C(CO)O[C@H](O[C@@H]6C(CO)O[C@@H](O[C@@H]7C(CO)O[C@H](O[C@@H]8C(CO)O[C@H](O[C@@H]9C(CO)O[C@H](O[C@H]1[C@H](O)C2O)C(O)[C@H]9O)C(O)[C@H]8O)C(O)[C@H]7O)C(O)[C@H]6O)C(O)[C@H]5O)C(O)[C@H]4O)C(O)[C@H]3O] |
| Cucurbituril | 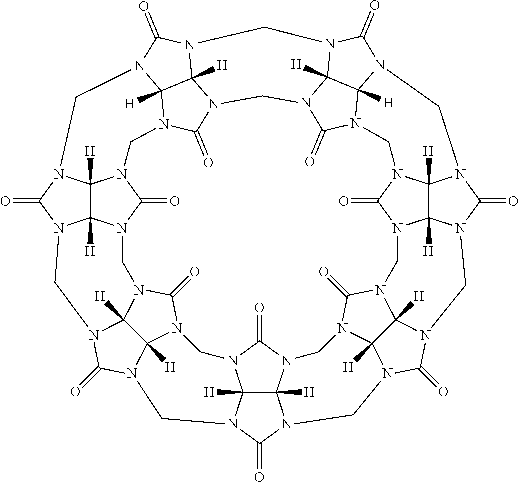 |

2. Branching Units Exemplary chemical structures suitable as branching units include monosaccharides. In some embodiments, the monosaccharide branching units are conjugated to the core or the prior layer of monomers via linkers such as polyethylene glycol chains. In preferred embodiments, the monosaccharide branching units are glucose-based branching units. Exemplary glucose-based branching units are shown in Structures II-IV. These are spacer molecules, so can also be alkyl (CH2)n-hydrocarbon-like units.

The branching units are the PEG or alkyl chain linkers between different dendrimer generations, for example, the glucose layers are connected via PEG linkers and triazole rings.

In preferred embodiments, the branching units are the same for each generation of dendrimers generated from the core. Therefore, in one embodiment, the branching units are glucose-based branching units for generating generation 1 dendrimers as shown in Structures V-VII.

In some embodiments, the branching units are hyper-monomers i.e., ABₙ building blocks. Exemplary hyper-monomers include AB₄, AB₅, AB₆, AB₇, AB₈ building blocks. Hyper-monomer strategy drastically increases the number of available end groups. An exemplary AB₄ hypermonomer is peracetylated β-D-Glucopyranoside tetraethylene glycol azide as shown in Structure III.

The chemical structures listed in Table 1, are also suitable as building blocks to form the branching units of the dendrimer. For example, the branching units of the dendrimers are formed by dipentaerythritol, pentaerythritol, 2-(aminomethyl)-2-(hydroxymethyl) propane-1,3-diol, 2-ethyl-2-(hydroxymethyl) propane-1,3-diol, 3,3′,3″,3′-silanetetrayltetrakis (propane-1-thiol), 3,3-divinylpenta-1,4-diene, 3,3′,3″-nitrilotripropionic acid, 3,3′,3″-nitrilotris(N-(2-aminoethyl)propanamide), 3,3′,3″,3′″-(ethane-1,2-diylbis(azanetriyl)) tetrapropanamide, 3-(carboxymethyl)-3-hydroxypentanedioic acid, 2,2′-((2,2-bis((2-hydroxyethoxy)methyl) propane-1,3-diyl)bis(oxy))bis(ethan-1-ol), tetrakis(3-(trichlorosilyl) propyl)silane, 1-Thioglycerol, 2,2,4,4,6,6-hexachloro-1,3,5,215,415,615-triazatriphosphinine, 3-(hydroxymethyl)-5,5-dimethylhexane-2,4-diol, 4,4′,4″-(ethane-1,1,1-triyl)triphenol, 2,4,6-trichloro-1,3,5-triazine, 5-(hydroxymethyl) benzene-1,2,3-triol, 5-(hydroxymethyl)benzene-1,3-diol, 1,3,5-tris(dimethyl(vinyl)silyl)benzene, Carbosiloxane core, nitrilotrimethanol, ethylene diamine, propane-1,3-diamine, butane-1,4-diamine, 2,2′,2″-nitrilotns(ethan-1-ol), alpha cyclodextrin, beta cyclodextrin, gamma cyclodextrin, Cucurbituril, benzene-1,2,3,4,5,6-hexathiol, monosaccharide, disaccharides, trisaccharides, oligosaccharides, or azide-, alkyne-modified moieties thereof, or a combination thereof.

Other examples of chemical structures that are suitable for forming the branching units of the dendrimers disclosed herein include, but are not limited to, sugar moieties, such as glucose, galactose, mannose, and fructose, and alkylene glycol, such as ethylene glycol, and combinations thereof. In some embodiments, the branching unit is chitosan. Thus, azide-modified chitosan, or alkyne-modified chitosan are suitable for conjugating to the core moiety or additional same or different branching units using click chemistry. In some embodiments, the branching unit is methyl acrylate or ethylenediamine, or a combination thereof. In some embodiments, the branching unit is polyethylene glycerol linear or branched. In some embodiments, the branching unit is a copolymer of an alkylene glycol (such as ethylene glycol) and a sugar moiety, such as glucose, galactose, mannose, and/or fructose.

### 3. Surface Functional Groups

Surface functional groups/molecules of the dendrimers are not limited to a primary amine end group, a hydroxyl end group, a carboxylic acid end group, an acetamide end group, a sugar molecule, an oligo- or poly-alkylene glycol, and/or a thiol end group. In some embodiments, the desired terminal functional groups can be added via one of the conjugation methods for the core and branching unit.

In some embodiments, the surface functional groups are hydroxyl groups, for example those of PAMAM dendrimers, of generation 2 PEG dendrimer as shown in Structure I, or of the terminal glucose of dendrimers prepared with glucose-based branching units as shown in Structures V and VII. In some embodiments, desired surface functional groups can be modified or added via one of the conjugation methods for the core and branching unit. Exemplary surface functional groups include hydroxyl end groups, amine end groups, carboxylic acid end groups, acetamide end group, and thiol end groups, and combinations thereof.

In some embodiments, the dendrimers can specifically target a particular tissue region and/or cell type, such as the cells and tissues of the central nervous system (CNS), the peripheral nervous system (PNS), and/or the eye. In some embodiments, the dendrimers specifically target neurons and/or glia of the CNS. In some embodiments, the dendrimers specifically target neurons and/or glia of the PNS. In some embodiments, the glucose dendrimers are those of generation 1 (G1), G2, G3, G4, and G5.

In some embodiments, the dendrimers include an effective number of terminal glucose and/or hydroxyl groups for targeting to one or more neurons and/or glia of the CNS, the PNS, and/or the eye.

Glucose dendrimers are preferred. In some embodiments, dendrimers are made of glucose and oligoethylene glycol building blocks. Exemplary generation 1 glucose dendrimer is shown in Structure VI, and generation 2 glucose dendrimers is shown in Structure VIII.

In some embodiments, the dendrimers have a plurality of surface functional groups, such as hydroxyl (—OH) groups, amine groups, acetamide groups, and/or carboxyl groups on the periphery of the dendrimers (also referred to herein as surface functional groups or peripheral functional groups). In some embodiments, the surface density of such peripheral functional groups is at least 1 group/nm² (number of the surface functional groups/surface area in nm²). For example, in some embodiments, the surface density of the surface functional groups, such as hydroxyl groups, is more than 0.5, 1, 2, 3, 4, 5, 6, 7, 8, 9, 10 OH groups/nm² such as at least 10, 15, 20, 25, 30, 35, 40, 45, 50, or more than 50 OH group/nm². In some embodiments, the volumetric density of surface functional groups, such as hydroxyl groups, is between about 1 and about 50 groups/nm³, between about 5 and about 30 groups/nm³, or between about 10 and about 20 groups/nm³. In further embodiments, the surface density of the surface functional groups, such as hydroxyl groups, is between about 1 and about 50, preferably 5-20 group/nm² (number of surface functional groups/surface area in nm²) while each surface functional moiety has a molecular weight of between about 100 Da and about 10 kDa, preferably between about 100 Da and 1000 Da.

In some embodiments, the amount of the surface functional groups, such as any one of those described above, e.g., hydroxyl groups, of the dendrimer is at least 40%, at least 50%, more than 40%, more than 50%, or in a range from more than 40% to 100%.

In some embodiments, one or more of the surface functional groups, such as any one of those described above, on the periphery of the dendrimers are further modified by conjugating with one or more carbohydrate molecules and/or more or more polyalkylene glycols, such as polyethylene glycols. In these embodiments, the surface density of the terminal carbohydrate moieties/molecules and/or polyalkylene glycols can be in any of the ranges described above for hydroxyl groups. Hydroxyl-terminated PAMAM dendrimers, PAMAM dendrimer modified on the surface with sugar moieties (with >10% of surface groups modified by sugars, especially by glucose, and glucose dendrimers (where the dendrimers are made of glucose building blocks) are preferred. For delivery to the brain, constructs with a total molecular weight of <30,000 Da are preferred. For confinement primarily to the peripheral circulation, constructs with a total molecular weight of >50,000 Da are preferred. When dendrimers are formed of, or include sugar moieties/molecules at termination, for example, glucose, the terminal hydroxyl groups of these dendrimers may be part of the terminal sugar moieties/molecules or extra hydroxyl groups that are not part of the sugar moieties/molecules, or a combination thereof. In some embodiments, all of the terminal hydroxyl groups are part of the terminal sugar moieties/molecules.

a. Hydroxyl-Terminated Dendrimers

In some embodiments, the dendrimers include a plurality of hydroxyl groups. Some exemplary high-density hydroxyl groups-containing dendrimers include commercially available polyester dendritic polymer such as hyperbranched 2,2-Bis(hydroxyl-methyl)propionic acid polyester polymer (for example, hyperbranched bis-MPA polyester-64-hydroxyl, generation 4), dendritic polyglycerols. In some embodiments, the hydroxyl terminated dendrimers include hydroxyl-terminated PAMAM dendrimers, particularly G3 to G6 hydroxyl-terminated PAMAM dendrimers, such as G3, G4, G5, and G6 hydroxyl-terminated PAMAM dendrimers.

In some embodiments, the high-density hydroxyl groups-containing dendrimers are oligo ethylene glycol (OEG)-like dendrimers. For example, a generation 2 OEG dendrimer (D2-OH-60) as shown in Structure I can be synthesized using highly efficient, robust and atom economical chemical reactions such as Cu (I) catalyzed alkyne-azide click and photo catalyzed thiol-ene click chemistry. Highly dense polyol dendrimer at very low generation in minimum reaction steps can be achieved by using an orthogonal hypermonomer and hypercore strategy, for example as described in WO2019094952. In some embodiments, the dendrimer backbone has non-cleavable polyether bonds throughout the structure to avoid the disintegration of dendrimer in vivo and to allow the elimination of such dendrimers as a single entity from the body (non-biodegradable).

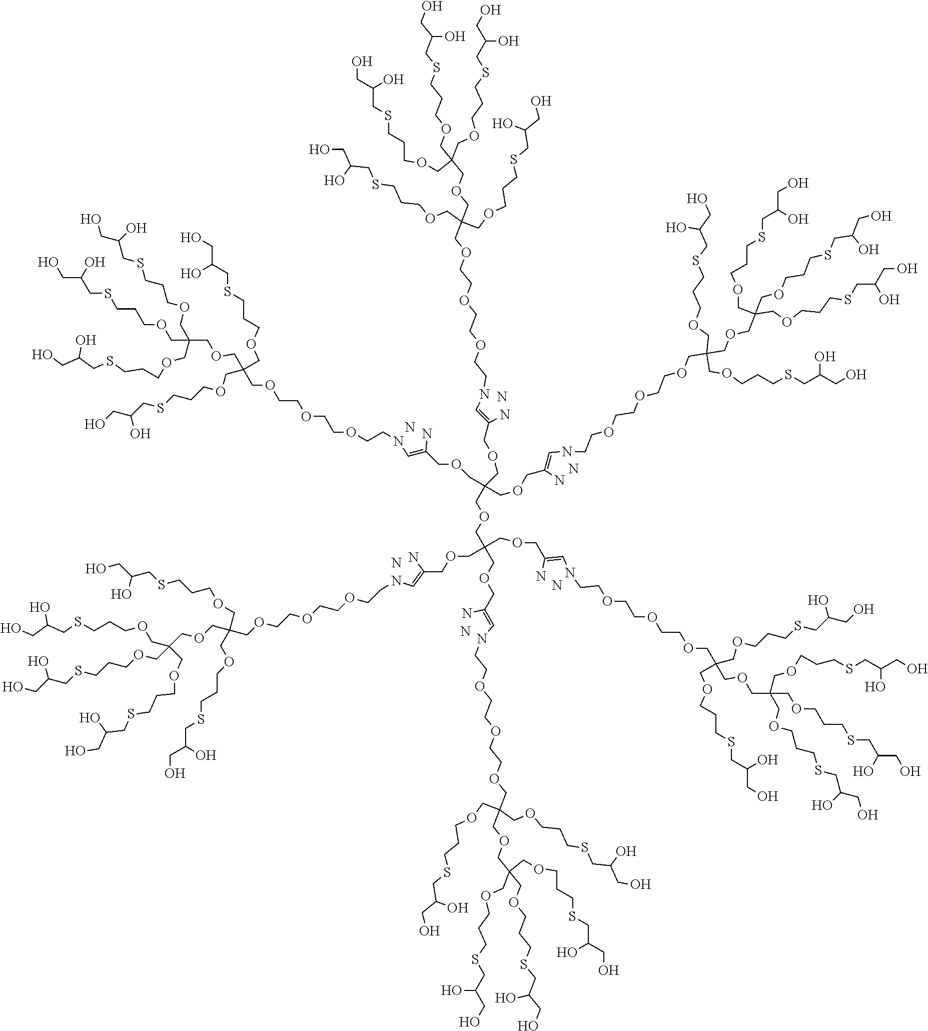

In some embodiments, the dendrimers have a plurality of hydroxyl 5 (—OH) groups on the periphery of the dendrimers. In some embodiments, the surface density of hydroxyl (—OH) groups is at least 1 OH group/nm² (number of surface hydroxyl groups/surface area in nm²). For example, in some embodiments, the surface density of hydroxyl groups, per nm², is more than 0.5, 1, 2, 3, 4, 5, 6, 7, 8, 9, 10 OH groups/nm², such as at least 10, 15, 20, 25, 30, 35, 40, 45, 50, or more than 50. In some embodiments, the volumetric density of hydroxyl groups is between about 1 and about 50 groups/nm³, between about 5 and about 30 groups/nm³, or between about 10 and about 20 groups/nm³. In further embodiments, the surface density of hydroxyl (—OH) groups is between about 1 and about 50, or between 5 and 20 OH group/nm² (number of surface hydroxyl groups/surface area in nm²) while having a molecular weight of between about 100 Da and about 10 kDa, preferably between about 100 Da and 1000 Da. In some embodiments, the amount of the surface hydroxyl groups of the dendrimer is preferably greater than 35%, at least 40%, at least 50%, more than 40%, more than 50%, or in a range from more than 40% to 100%. In some embodiments, the dendrimers may have a fraction of the hydroxyl groups exposed on the outer surface, with the others in the interior core of the dendrimers.

In some embodiments, the dendrimer specifically targets a particular tissue region and/or cell types following administration into the body. In some embodiments, the dendrimer specifically targets a particular tissue region and/or cell type without a targeting moiety. In some embodiments, the dendrimers include an effective number of hydroxyl groups for targeting CNS cells and/or PNS cells, such as microglial, astrocytes, and/or neurons associated with a disease, disorder, or injury of the central nervous system or the peripheral nervous system. In some embodiments, the dendrimer specifically targets a particular tissue region and/or cell type without a targeting moiety and the active agent conjugated thereto bind directly to a receptor on the surface and/or interior of target neural and/or glial cells.

In some embodiments, the dendrimers are able to specifically target a particular tissue region and/or cell type, preferably the cells and tissues of the central nervous system (CNS) and the eye. In some embodiments, the dendrimers specifically target neurons of the CNS and the eye. Unmodified PAMAM dendrimers with hydroxyl end groups do not enrich in the neurons of brain and/or retinal ganglion cells (RGCs) in the eye as much as these glucose dendrimers. The glucose dendrimers with terminal glucose monosaccharide and a high density of hydroxyl functional groups effectively target the neurons in a generation dependent manner. Examples demonstrate efficacy with generation 2 (G2), and G3 and G4 should be efficacious. G5 and above are more difficult to use.

In preferred embodiments, the dendrimers include an effective number of terminal glucose and/or hydroxyl groups for targeting to one or more neurons of the CNS, or the eye. The hydroxyl groups on the dendrimer surface are part of glucose molecules. There are no extra hydroxyls in addition to the glucose molecules on the surface. The number of sugar molecules on the surface is determined by the generation number. All generations are expected to target neurons.

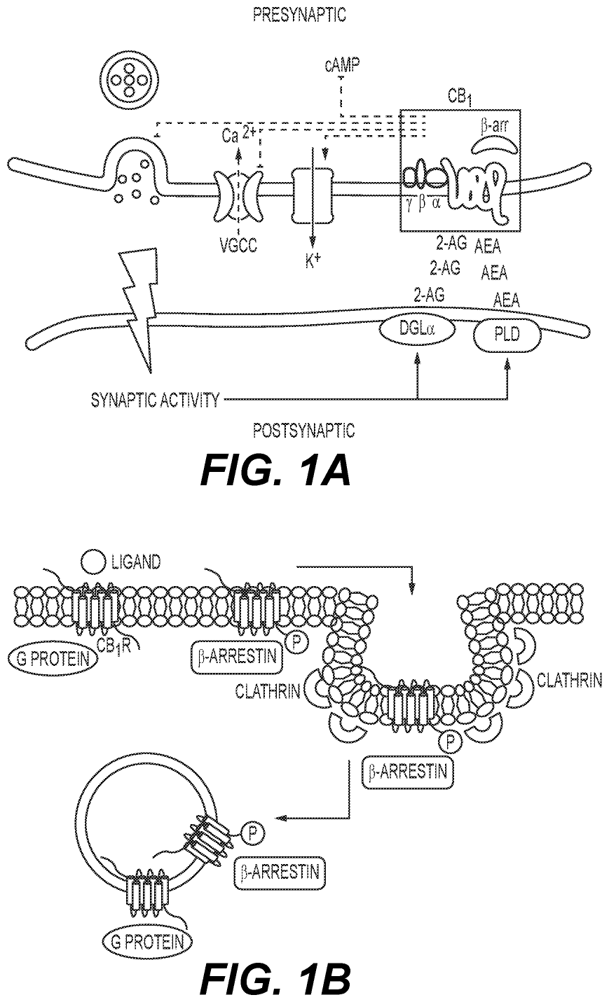

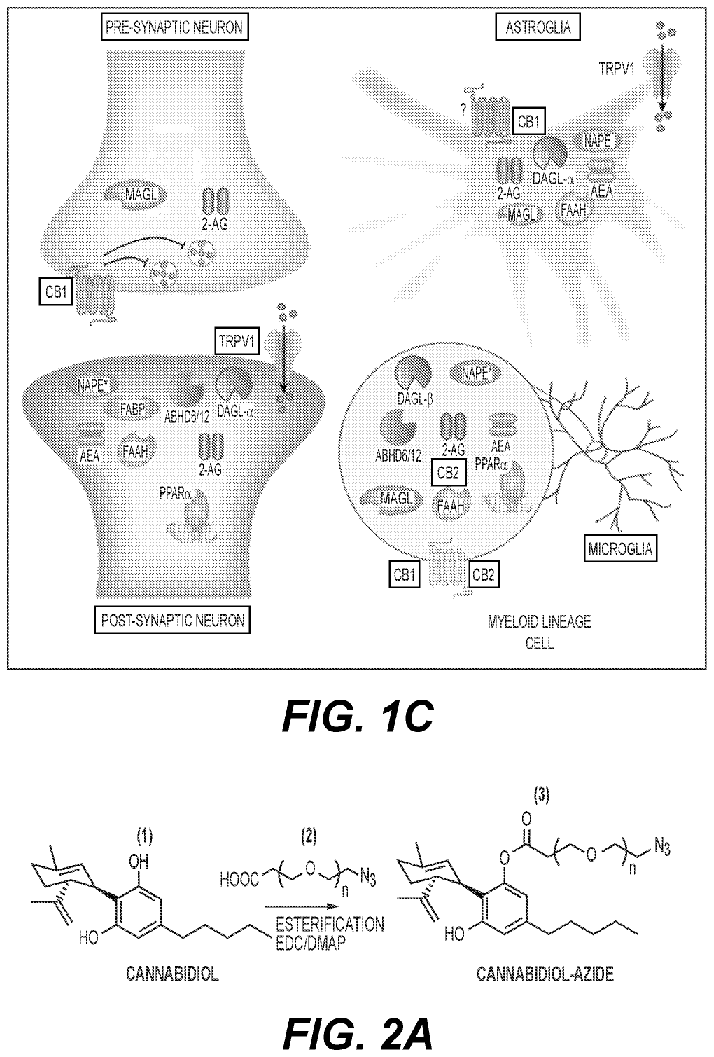

In some embodiments, dendrimers are made of glucose and oligoethylene glycol building blocks. Exemplary glucose dendrimers are shown in the Examples, for example, generation 1 dendrimers as shown in Structures IV-VI, and generation 2 dendrimers as shown in FIGS. 1A and 1B. Some exemplary glucose dendrimers include a generation 1 glucose dendrimer having 24 hydroxyl (—OH) end groups, a generation 2 glucose dendrimer having 96 hydroxyl (—OH) end groups, a generation 3 glucose dendrimer having 396 hydroxyl (—OH) end groups, and generation 4 glucose dendrimer having 1584 hydroxyl (—OH) end groups. In a preferred embodiment, the glucose dendrimer is a generation 2 glucose based dendrimer that has 24 glucose molecules at the periphery and 6 embedded glucose molecules in the backbone held together by PEG segments.

b. Dendrimers Modified with Carbohydrates

In some embodiments, the dendrimers contain one or more carbohydrate molecules at the termination. These terminal carbohydrate molecules can be prepared by conjugating one or more surface functional groups of a dendrimers, such as amine groups, carboxyl groups, or hydroxyl groups, with one or more carbohydrate molecules. In preferred embodiments, the dendrimers, prior to carbohydrate conjugation, are hydroxyl-terminated dendrimers such as hydroxyl-terminated PAMAM dendrimers and one or more of the hydroxyl groups are conjugated with one or more carbohydrate molecules.

In some embodiments, hydroxyl-terminated dendrimers modified with surface glucose molecules selectively target central and/or peripheral neural and/or glial cells in vitro and in vivo; and/or selectively accumulate on the surface and/or within these target neural cells, glial cells, and/or macrophage cells, such that the active agent(s) conjugated thereto bind to one or more receptors on/in the target neural and/or glial cells.

In some embodiments, the carbohydrate moieties used to modify one or more surface functional groups of the dendrimers are monosaccharides. Exemplary monosaccharides suitable for modifying the dendrimers include glucose, glucosamine, galactose, manmose, fructose, dehydroascorbic acid, urate, myo-inositol. In some embodiments, the dendrimers are conjugated to glucose and thus contain glucose as terminal moieties/molecules. In some embodiments, hydroxyl-terminated dendrimers are modified with one or more glucose moieties to the dendrimer (“D-Glu”). In some embodiments, the dendrimers are conjugated to galactose. In some embodiments, the dendrimers are conjugated to mannose. In some embodiments, the dendrimers are conjugated to fructose. In some embodiments, the dendrimers are conjugated to one or more monosaccharides other than glucose, such as galactose, mannose, and/or fructose. For example, the carbohydrate moieties are oligosaccharides which terminate in one or more monosaccharides including glucose, glucosamine, mannose, fructose, thus exposing these sugar moieties on the surface for binding.

The glucose dendrimers or glucose-modified dendrimers are used to obtain selective uptake by the target cells. The drug conjugated to the dendrimer binds to receptors or other sites of action. In preferred embodiments, the dendrimers or functionally modified dendrimers are conjugated to one or more drugs that have affinity to and are suitable for binding one or more of cannabinoid receptors e.g., CB₁ receptors, CB₂ receptors, and CB₃ receptors. In some embodiments, the dendrimers are conjugated to one or more carbohydrates moieties that have affinity to and are suitable for binding one or more non-cannabinoid receptors such as G-protein coupled receptors e.g., GPR55, GPR18, GPR3, GPR6, GPR12 GPR40, GPR43, GPR41, GPR120, GPR23, GPR92, GPR84, GPR119, or GPR35; the adenosine receptor such as adenosine A3; the muscarinic acetylcholine receptors e.g., M1 and M4; the serotonin receptors e.g., 5-HT1A, 5-HT2A; opioid receptors e.g., μ- and δ-opioid receptors; and tachykinin NK2 receptors. In some embodiments, the dendrimers are conjugated to one or more carbohydrates moieties, or made of sugar moieties, that have affinity to and are suitable for transport via one or more of GLUT1, GLUT2, GLUT3, GLUT4, GLUT5, GLUT6, GLUT7, GLUT8, GLUT9, GLUT10, GLUT11, GLUT12, GLUT13, and GLUT14. In further embodiments, the dendrimers are conjugated to one or more glucose and/or glucosamine moieties. In other embodiments, the dendrimers are conjugated to one or more oligosaccharides terminating in glucose and/or glucosamine moieties, i.e., glucose and/or glucosamine moieties are exposed on the surface of the dendrimer conjugates suitable for binding to one or more of the GLUTs, cannabinoid receptors and/or non-cannabinoid receptors.

In some embodiments, the dendrimers have a plurality of carbohydrate moieties/molecules such as monosaccharides, e.g., glucose, on the periphery of the dendrimers, or have sugar building blocks for the dendrimers. In some embodiments, the surface density of carbohydrate molecules such as monosaccharides, e.g., glucose, is at least 1 carbohydrate molecule/nm² (number of surface carbohydrate groups/surface area in nm²). In some embodiments, the surface density of carbohydrate molecules, per nm², is more than 0.5, 1, 2, 3, 4, 5, 6, 7, 8, 9, 10 OH groups/nm², such as at least 10, 15, 20, 25, 30, 35, 40, 45, 50, or more than 50. For example, surface density of carbohydrate molecules, per nm², is more than 10. In some embodiments, the volumetric density of surface carbohydrate molecules is between about 1 and about 50 groups/nm³, between about 5 and about 30 groups/nm³, or between about 10 and about 20 groups/nm³. In further embodiments, the surface density of carbohydrate molecules is between about 1 and about 50, between about 5 and about 20, per nm² (number of surface carbohydrate molecules/surface area in nm²) while each carbohydrate moiety having a molecular weight of between about 100 Da and about 1000 Da. In these embodiments, i.e., one or more surface functional groups of the dendrimer are modified to introduce one or more sugar moieties/molecules at termination, the terminal hydroxyl groups may be part of the terminal sugar moieties/molecules or extra hydroxyl groups that are not modified with sugar moieties/molecules and thus are not part of the sugar moieties/molecules, or a combination thereof.

In some embodiments, carbohydrate molecules such as monosaccharides, e.g., glucose, are present in an amount by weight that is between about 1% and 40% of the total weight of the glycosylated dendrimer, for example, between about 2% and 20%, between about 5% and 15%, or between 9% and 12% of the total weight of the glycosylated dendrimer. For example, in some embodiments, the carbohydrate moieties are present in an amount that is about 1%, 2%, 3%, 4%, 5%, 6%, 7%, 8%, 9%, 10%, 11%, 12%, 13%, 14%, 15%, 16%, 17%, 18%, 19%, or 20% of the total weight of the glycosylated dendrimer following conjugation. In some embodiments, conjugation of carbohydrate molecules through one or more surface functional groups occurs via about 5%, 6%, 7%, 8%, 9%, 10%, 15%, 20%, or 25% of the total available surface functional groups, preferably hydroxyl groups, of the dendrimers prior to the conjugation. In other embodiments, the conjugation of carbohydrate molecules occurs on less than 5%, less than 10%, less than 15%, less than 20%, less than 25%, less than 30%, less than 35%, less than 40% of total available surface functional groups of the dendrimers prior to the conjugation.

c. Dendrimers Modified with Polyalkylene Glycol

In some embodiments, the dendrimers contain one or more polyalkylene glycols at the termination. These terminal polyalkylene glycols can be prepared by conjugating one or more of surface functional groups of the dendrimers, such as hydroxyl groups, with a polyalkylene glycol, such as PEG. In some embodiments, the dendrimers, prior to conjugation, are hydroxyl-terminated dendrimers such as hydroxyl-terminated PAMAM dendrimers and at least a portion of the surface hydroxyl groups are conjugated with PEG.

In some embodiments, the dendrimers have a plurality of polyalkylene glycols such as PEG, on the periphery of the dendrimers. In some embodiments, the surface density of polyalkylene glycols such as PEG, is at least 1 polyalkylene glycol/nm² (number of surface polyalkylene glycol/surface area in nm²). In some embodiments, the surface density of polyalkylene glycols, per nm², is more than 0.5, 1, 2, 3, 4, 5, 6, 7, 8, 9, 10, such as at least 10, 15, 20, 25, 30, 35, 40, 45, 50, or more than 50. For example, surface density of polyalkylene glycols, per nm², is more than 10. In some embodiments, the volumetric density of surface polyalkylene glycols is between about 1 and about 50 groups/nm³, between about 5 and about 30 groups/nm³, or between about 10 and about 20 groups/nm³. In further embodiments, the surface density of polyalkylene glycols such as PEG is between about 1 and about 50, between about 5 and about 20, per nm² (number of surface polyalkylene glycols/surface area in nm²) while having a molecular weight of between about 100 Da and about 10 kDa.

In some embodiments, the polyalkylene glycol molecules such as PEG can be present in an amount by weight that is between about 10% and 40% of the total weight of the pegylated dendrimer, for example, between about 2% and 20%, between about 5% and 15%, or between 9% and 12% of the total weight of the pegylated dendrimer. For example, in some embodiments, the polyalkylene glycol molecules, such as PEG, are present in an amount that is about 1%, 2%, 3%, 4%, 5%, 6%, 7%, 8%, 9%, 10%, 11%, 12%, 13%, 14%, 15%, 16%, 17%, 18%, 19%, or 20% of the total weight of the pegylated dendrimer following conjugation.

In some embodiments, conjugation of polyalkylene glycol molecules such as PEG through one or more surface functional groups of the dendrimer occurs via about 5%, 6%, 7%, 8%, 9%, 10%, 15%, 20%, or 25% of the total available surface functional groups, preferably hydroxyl groups, of the dendrimers prior to the conjugation. In other embodiments, the conjugation of polyalkylene glycol molecules such as PEG occurs on less than 5%, less than 10%, less than 15%, less than 20%, less than 25%, less than 30%, less than 35%, less than 40% of total available surface functional groups of the dendrimers prior to the conjugation.

### B. Cannabinoids

The dendrimer compositions typically include one or more cannabinoids and/or cannabinoid derivatives conjugated to the dendrimer. Exemplary cannabinoids that may be included in the compositions belong to one or more classes of cannabinoids, including, but not limited to, plant-derived cannabinoids, endogenous cannabinoids, and synthetic cannabinoids. The cannabinoids or cannabinoid derivatives included in the compositions typically bind to one or more receptors, thereby modulating signaling in the endocannabinoid system. Many of the effects of cannabinoids and cannabinoid derivatives are mediated by two G protein-coupled receptors (GPCRs), CB1 and CB2, although additional receptors may be involved. CB1 receptors are present in high levels in several brain regions and in lower amounts in the periphery. CB1 receptors mediate many of the psychoactive effects of cannabinoids. CB2 receptors have a more restricted distribution, being found in a number of immune cells and in a few neurons. Both CB1 and CB2 receptors couple primarily to inhibitory G proteins and are subject to the same pharmacological influences as other GPCRs. Thus, partial agonism, functional selectivity and inverse agonism all play important roles in determining the cellular response to specific cannabinoid receptor ligands. Human Cannabinoid Type 1 and Type 2 receptor agonists and antagonists of interest, for example, include ARAP433912, Parecoxib, Valdecoxib, PSNCBAM-1, GAT 211, GAT 228, GAT-229, (±)19(20)-EDP Ethanolamide. Voacamine, LY320135, Falcarinol, Lasofoxifene, ABD459, AM6545, PSB-SB1202, MCHB-1, KM 233, CB-25, CB-52, Leeamine, and Docosahexaenoyl Ethanolamide.

### 1. Endocannabinoids

The compositions may include one or more endogenous cannabinoids or endocannabinoids. Endocannabinoids are lipid-signaling molecules that are produced from within the body that activate cannabinoid receptors and mimic the activity of Δ9-tetrahydrocannabinol, the main psychotropic constituent of cannabis. Exemplary endocannabinoids and endocannabinoid analogs that may be included in the compositions include, but are not limited to, N-arachidonoylethanolamide (anandamide), 2-arachidonoylglycerol (2-AG), 2-Arachidonyl glyceryl ether (noladin ether), N-Arachidonoyl dopamine (NADA), Virodhamine (OAE), Lysophosphatidylinositol (LPI), 7,10,13,16-docosatetraenoylethanolamide and homo-γ-linolenoylethanolamine. Not all are readily conjugable, but modifications can be made to most to enable conjugation.

In some forms, the endocannabinoid included in the composition binds to both the CB1 and CB2 receptors with similar affinity, acting as an agonist at both the CBT and CB2 receptors. For example, 2-arachidonoylglycerol binds to both the CB1 and CB2 receptors with similar affinity, acting as a full agonist at both receptors.

In some forms, the endocannabinoid included in the composition binds with high affinity to the CB1 receptor, and weak affinity for the CB2 receptor. For example, anandamide, 7,10,13,16-docosatetraenoylethanolamide and homo-γ-linolenoylethanolamine, bind to the central (CB1) and, to a lesser extent, peripheral (CB2) cannabinoid receptors, where they act as partial agonists. In another example, 2-Arachidonyl glyceryl ether (noladin ether) binds primarily to the CBT receptor, and only weakly to the CB2 receptor, thereby inducing sedation, hypothermia, intestinal immobility, anti-nociception. In yet a third example, N-Arachidonoyl dopamine preferentially binds to the CBT receptor.

In some forms, the endocannabinoid included in the composition binds with high affinity to the CB2 receptor, and weak affinity for the CB1 receptor. For example, virodhamine, or O-arachidonoyl-ethanolamine (OAE), binds with strong affinity to the CB2 receptor, and acts as a partial agonist at CB1 receptors.

In some forms, the endocannabinoid included in the composition binds to one or more non CB1/CB2 receptors. For example, anandamide and NADA act as strong agonists for the vanilloid receptor subtype 1 (TRPV1), a member of the vanilloid receptor family. In another example, the endogenous lipid metabolite of anandamide (also known as arachidonoylethanolamide or AEA), binds to the N-arachidonoyl glycine (NAGly) receptor GPRT8, an orphan G-protein-coupled receptor.

### 2. Phytocannabinoids

The compositions may include one or more phytocannabinoids or phytocannabinoid derivatives. Phytocannabinoids or phytocides are a structurally diverse class of naturally occurring chemical constituents derived from plants. In some forms, the phytocannabinoids or phytocannabinoid derivatives used in the compositions may be derived from various sources, including but not limited to hemp (e.g. hemp stalk, hemp stem, hemp seed), cannabis (e.g., cannabis flower, cannabis leaf, cannabis stalk, cannabis stem, cannabis seed), Echinacea purpurea, Echinacea angustifolia, Echinacea pallida, Acmella oleracea, Helichrysum umbraculigerum, Radula marginata, kava, black truffle, Syzygium aromaticum (cloves), Rosmarinus oficinalis, basil, oregano, black pepper, lavender, true cinnamon, malabathrum, cananga odorata, copaifera spp., and hops. Examples include THC, Δ9-tetrahydrocannabinol; Δ9-THCV, Δ9-tetrahydrocannabivarin; CBN, cannabinol; CBDV, cannabidivarin; CBG, cannabigerol; CBC, and cannabichromene.

The compositions may contain one or more phytocannabinoids or phytocannabinoid derivatives belonging to one or more of the following subclasses: cannabidiols (CBDs), tetrahydrocannabinols (THCs), cannabigerols (CBGs), cannabinols (CBNs), cannabichromenes (CBCs), cannabielsoins (CBEs), cannabicyclols (CBLs), cannabifurans (CBFs), cannabitran (CBTs), Cannabinodivarins (CBVs), cannabiripsol (CBR), isocannabinoids and/or derivatives thereof.

a. Cannabidiol

In some forms, the compositions may contain one or more cannabidiols (CBDs) and/or their derivatives. Suitable examples of cannabidiols (CBDs) include but are not limited to Cannabidiol (CBD), Cannabidiolic Acid (CBDA), Cannabidiorcol (CBDC1), Cannabidiol-C4 (CBDC4), Cannabidiol dimethyl ether (CBDD), Cannabidiol Monomethylether (CBDM), Cannabidivarin (CBDV), Cannabidivarinic Acid (CBDVA), and derivatives thereof. In some forms, the compositions may contain one or more cannabinodiols (CBNDs) and/or their derivatives. Cannabinodiols or cannabidinodiols are typically present at low concentrations in the plant Cannabis sativa. Cannabinodiols are the fully aromatized derivative of cannabidiol (CBD) and can occur as a product of the photochemical conversion of cannabinol (CBN). In one preferred embodiment, the dendrimer is conjugated to one or more cannabidiols (CBD) or cannabidiol derivatives as shown in Formula I below.

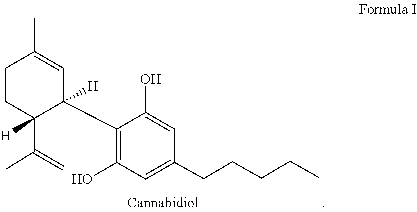 [SMILES: C=C(C)[C@@H]1CCC(C)=C[C@H]1c1c(O)cc(CCCCC)cc1O]

b. Tetrahydrocannabinol

In some forms, the compositions may contain one or more tetrahydrocannabinols (THCs). Non-limiting examples of tetrahydrocannabinols include Tetrahydrocannabinol, (THC), 10-oxo-delta-6a-tetrahydrocannabinol, (OTHC), Delta-8-tetrahydrocannabinolic acid, (A8-THCA), Delta-8-tetrahydrocannabinol (d8THC, A8-THC), Delta-9-tetrahydrocannabinol (d9THC, Δ9-THC), Delta-9-tetrahydrocannabinol-C4 (THC-C4), Delta-9-tetrahydrocannabinolic acid A (Δ9-THCA, THCA-C5), Delta-9-tetrahydrocannabinolic acid B (Δ9-THCB, THCA-B), Delta-9-tetrahydrocannabiorolic acid, (Δ9-THCA-C1, THCA-C1), Delta-9-tetrahydrocannabinolic acid C4, (Δ9-THCA-C4, THCA-C4), Tetrahydrocannabinol(−)-cis-A (THC-C5), Delta-9-tetrahydrocannabiorcol (Δ9-THCO-C1), Delta-9-Tetrahydrocannabiorcolic acid A (THCOA), Delta-9-tetrahydrocannabivarin (THCV), Delta-9-tetrahydrocannabivarinic acid A (THCVA), TriHydroxy-delta-9-tetrahydrocannabinol (TRIOH-THC), Delta-10-tetrahydrocannabinol (D10THC, AlOTHC), Tetrahydrocannabiphorol (THCP), THC-O Acetate (THCO), Hexahydrocannabinol (HHC), and derivatives thereof. In a second preferred embodiment, the dendrimer is conjugated to one or more tetrahydrocannabinols (THC), or THC derivatives as shown in Formula II below.

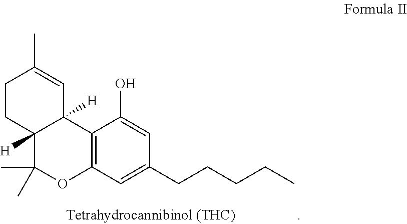 [SMILES: CCCCCc1cc(O)c2c(c1)OC(C)(C)[C@@H]1CCC(C)=C[C@@H]21]

c. Cannabigerol (CBG)

In some forms, the compositions may contain one or more cannabigerols (CBGs) or derivatives thereof. Suitable examples of cannabigerols include but are not limited to Cannabigerolic Acid (CBGA), Cannabigerolic Acid A Monomethylether (CBGAM), Cannabigerovarin (CBGV), Cannabigerovarinic Acid (CBGVA), Cannabigerol Monomethylether (CBGM), and derivatives thereof. In a third preferred embodiment, the dendrimer is conjugated to one or more cannabigerols (CBG), or CBG derivatives as shown in Formula III below.

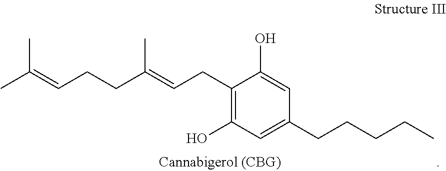 [SMILES: CCCCCc1cc(O)c(C/C=C(\C)CCC=C(C)C)c(O)c1]

d. Cannabinols (CBNs)

In some forms, the compositions may contain one or more cannabinols (CBNs) and/or their derivatives. Suitable examples of cannabinols include but are not limited to Cannabinol (CBN), Cannabinolic Acid (CBNA), Cannabidiorcol (CBN-C1), Cannabinol-C2 (CBN-C2), Cannabivarin (CBN-C3), Cannabinol-C4 (CBN-C4), Cannabinodiol (CBND), Cannabinodivarin (CBNDC3), Cannabinol Methyl ether (CBNM-C5), Delta-9-cis-tetrahydrocannabinol (CIS-THC), and their derivatives. In a fourth preferred embodiment, the dendrimer is conjugated to one or more cannabinols (CBN) or cannabinol derivatives as shown in Formula IV below.

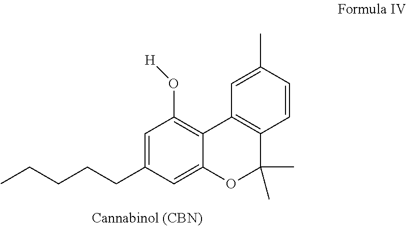 [SMILES: CCCCCc1cc(O)c2c(c1)OC(C)(C)c1ccc(C)cc1-2]

e. Cannabichromenes (CBCs)

In some forms, the compositions may contain one or more cannabichromenes (CBCs) and/or their derivatives. Non-limiting examples of cannabichromenes (CBCs) that may be included in the compositions include Cannabichromene (CBC), Cannabichromenic Acid A (CBCA), Cannabichromanon (CBCN-C5), Cannabichromanone-C3 (CBCN-C3), Cannabicoumaronone (CBCON-C5), Cannabichromevarin (CBCV), Cannabichromevarinic Acid A (CBCVA), and derivatives thereof. In a fifth embodiment, the dendrimer is conjugated to one or more cannabichromenes (CBCs) or CBC derivatives as shown in Formula V below.

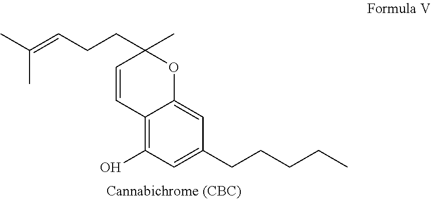 [SMILES: CCCCCc1cc(O)c2c(c1)OC(C)(CCC=C(C)C)C=C2]

f. Cannabielsoins (CBL)

In some forms, the compositions may include one or more cannabielsoins (CBL) or derivatives thereof. Cannabielsoin (CBE) Cannabielsoin (CBE) is a phytocannabinoid metabolite which can be produced by photo-oxidation from CBD and CBDA, or by biotransformation using tissue cultures under normal growth conditions. Non-limiting examples of cannabielsoins (CBL) include Cannabielsoin (CBE-C5), -C3-Cannabielsoin (CBE-C3), Cannabielsoic Acid A (CBEA-C5 A), Cannabielsoic Acid B (CBEA-C5 B), C3-Cannabielsoicacid B (CBEA-C3 B), CannabiglendolC3 (OH-iso-HHCVC3), Dehydrocannabifuran (DCBF-C5), Cannabifuran (CBF-C5), and their derivatives. In some forms, one or more cannabielsoins may be included in the compositions to modulate the activity of CB1 receptors, CB2 receptors, a non-CB1 receptors or a combination thereof. In an exemplary embodiment, the dendrimer is conjugated to one or more cannabielsoins (CBL) or CBL derivatives as shown in Formula VI below.

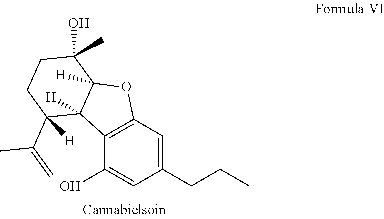

g. Cannabitriols (CBTs)

The compositions may include one or more cannabitriols (CBTs) and/or their derivatives. Cannabitriol is present in low concentration in the Cannabis plant and is an oxidation product of tetranhydrocannabinol (THC). Non-limiting examples of cannabitriols (CBTs) include Cannabitriol (CBT), Cannabitriolvarin (CBTV), 10-Ethoxy-9-hydroxy-delta-6a-tetrahydrocannabinol, 8,9-Dihydroxy-delta-6a-tetrahydrocannabinol (8,9-Di-OH), 9,10-Dihydroxyhexahydrocannabinol, Cannabiripsol, (Cannabiripsol-C5), 6a,7,10aTrihydroxyA9-tetrahydrocannabinol10-OxoA6a(10a)tetrahydrocannabinol, OTHC, Trans-Cannabitriol (Trans-CBT-C5), Cannabitriol-C3 (TransCBT-C3), Trans10-O-Ethylcannabitriol (TransCBT-OEt-C5), and their derivatives. In some forms, one or more cannabitriols may be included in the compositions to modulate the activity of CBT receptors, CB2 receptors, a non-CB1 receptors or a combination thereof. In an exemplary embodiment, the dendrimer is conjugated to one or more cannabitriols (CBTs) or derivatives thereof as shown in Formula VII below.

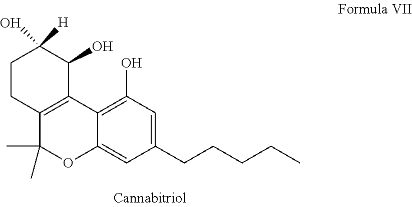 [SMILES: CCCCCc1cc(O)c2c(c1)OC(C)(C)C1=C2[C@H](O)[C@@H](O)CC1]

h. Other Phytocannabinoids

The compositions may include one or more other phytocannabinoids and/or their derivatives. In some forms, the compositions may include one or more cannabicyclol (CBL) or derivatives thereof. Cannabicyclol (CBL) is a photochemical product that originates from the phytocannabinoid cannabichromene. Suitable examples of cannabicyclol include but are not limited to Cannabicyclol (CBL-C5), Cannabicyclolic Acid (CBLA-C5 A), Cannabicyclovarin (CBLV-C3), and their derivatives. In some forms, the compositions may include one or more cannabifuran (CBF) or derivatives thereof. Examples of suitable cannabifurans include but are not limited to Cannabifuran (CBF), Dehydrocannabifuran (DCBF or CBFD) and their derivatives. In some forms, the compositions may include one or more Cannabinodivarins (CBVs) or derivatives thereof. Suitable examples of Cannabinodivarins (CBVs) include but are not limited to Cannabinodivarin (CBV), Cannabinodivarin (CBVD), and their derivatives. In some forms, the compositions may include one or more miscellaneous cannabinoids including but not limited to cannabiripsol (CBRs), isocannabinoids, and/or their derivatives. Non-limiting examples of isocannabinoids include, but are not limited to, Isotetrahydrocannabinol, Isotetrahydrocannabivarin, Isotetrahydrocannabivarin, and their derivatives.

i. Synthetic Cannabinoids

The compositions may include one or more synthetic cannabinoids and/or their derivatives.

Synthetic cannabinoids (also referred to as cannabinoid receptor agonists), are a class of designer drug molecules that are functionally similar to Δ9-tetrahydrocannabinol (THC) and bind to one or more cannabinoid and non-cannabinoid receptors in the central and peripheral nervous system similar to endocannabinoids and/or phytocannabinoids. However, synthetic cannabinoids are generally regarded as psychoactive substances and are structurally distinct from synthetic phytocannabinoids (such as THC or CBD obtained by chemical synthesis) and synthetic endocannabinoids. The cannabinoid receptor agonists form a diverse group, but most are lipid soluble and non-polar, and contain from about 22 to about 26 carbon atoms. A common structural feature of synthetic cannabinoids is a sidechain, where optimal activity requires more than four and up to nine saturated carbon atoms.

The compositions may contain one or more synthetic cannabinoids and/or their derivatives belonging to one or more of the following subclasses: classical cannabinoids, non-classical cannabinoids, hybrid cannabinoids, aminoalkylindoles, and eicosanoids. The compositions may contain one or more synthetic cannabinoids and/or their derivatives belonging to one or more of naphthoylindoles (e.g., JWH-018, JWH-073 and JWH-398); naphthylmethylindoles, naphthoylpyrroles, naphthylmethylindenes, phenylacetylindoles (i.e., benzoylindoles, e.g., JWH-250), cyclohexylphenols (e.g., CP 47,497 and homologues of CP 47,497), and classical cannabinoids (e.g., HU-210). The compositions may contain one or more synthetic cannabinoids and/or their derivatives belonging to one or more of adamantoylindoles or indazole carboxamids, benzimidazoles, dibenzopyrans, eicosanoids, naphtylindenes, indazole-3-carboxamides, indole-3-carboxamides, indole-3-carboxylates or aryloxycarbonylindoles, naphthoylindazoles, pyrazolecarboxamides, quinolinyl esters or aryloxycarbonylindole, tetramethylcyclo-propylcarbonylindazoles, and/or tetramethylcyclo-propylcarbonylindoles.

In an exemplary embodiment, the dendrimer is conjugated to one or more naphthoylindoles or derivatives thereof as shown in Formula VIII below, wherein R₁, R₂, R₃, and R₄ are positions at which substituent variants are possible.

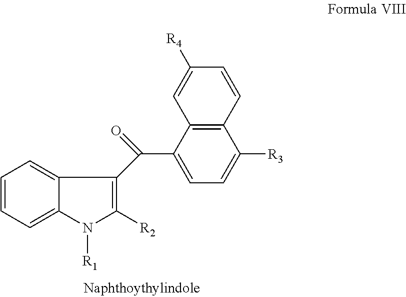

In another exemplary embodiment, the dendrimer is conjugated to one or more naphthylmethylindoles or derivatives thereof as shown in Formula IX below, wherein R₁, R₂, and R₃, are positions at which substituent variants are possible.

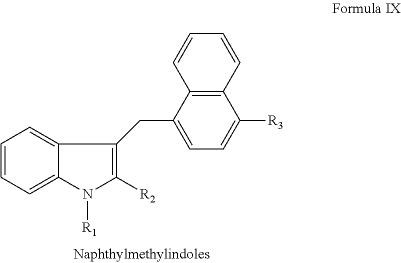

In another exemplary embodiment, the dendrimer is conjugated to one or more naphthoylpyrroles or derivatives thereof as shown in Formula X below, wherein R₁, and R₂ are positions at which substituent variants are possible.

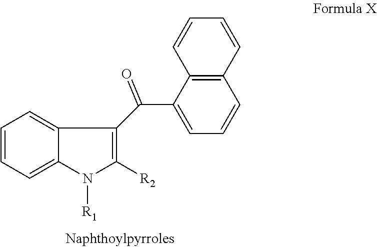

In another exemplary embodiment, the dendrimer is conjugated to one or more naphthylmethylindenes or derivatives thereof as shown in Formula XI below, wherein R₁, and R₂ are positions at which substituent variants are possible.

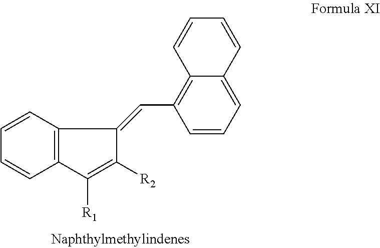

In another exemplary embodiment, the dendrimer is conjugated to one or more phenylacetylindoles or derivatives thereof as shown in Formula XII below, wherein R₁, and R₂ are positions at which substituent variants are possible.

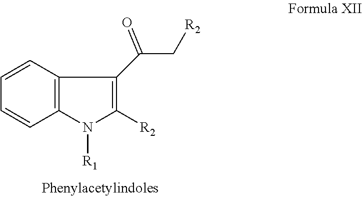

In another exemplary embodiment, the dendrimer is conjugated to one or more cyclohexylphenols or derivatives thereof as shown in Formula XIII below, wherein R₁, and R₂ are positions at which substituent variants are possible.

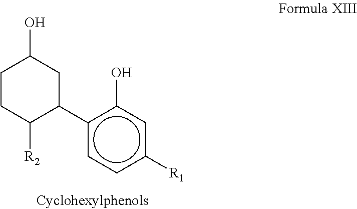

In another exemplary embodiment, the dendrimer is conjugated to one or more classical cannabinoids (dibenzopyrans) and/or derivatives thereof as shown in Formula XIV below, wherein R₁, and R₂ are positions at which substituent variants are possible.

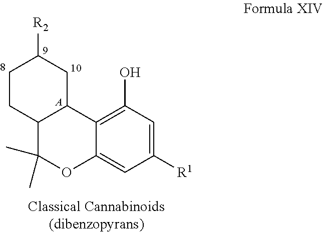

### C. Coupling Agents and Spacers

Dendrimer-active agent conjugates can be formed from one or more active agents covalently conjugated or non-covalently attached to a dendrimer. In preferred embodiments, the one or more active agents are covalently conjugated to the dendrimer.

Optionally, the one or more active agents are conjugated to the dendrimer via one or more spacers. The term “spacer” includes chemical moieties and functional groups used for linking an active agent to the dendrimer. The spacer can be either a single chemical entity or two or more chemical entities linked together. The spacer can include any small chemical entity, peptide or polymers having sulfhydryl, thiopyridine, succinimidyl, maleimide, vinylsulfone, carbonate, etc.

In some embodiments, the spacer via which the active agent is conjugated to the dendrimer contains different linkages such as disulfide, ester, carbonate, carbamate, thioester, hydrazine, hydrazides, ether, and amide linkages. The spacer between a dendrimer and an active agent can be designed to provide a releasable or non-releasable form of the dendrimer conjugate in vivo. In some embodiments, the conjugation between active agent and dendrimer is via an appropriate spacer that contains an ester bond between the active agent and the dendrimer. In some embodiments, one or more spacers between a dendrimer and active agents can provide desired and effective release kinetics in vivo. These spacers may contain cleavable linkages (e.g., ester, disulfide, phosphodiester, triglycyl peptide, and hydrazine) or non-cleavable linkages (e.g., amide, ether, and amino alkyl). The conjugation between active agents and dendrimers can be performed using reaction known in the art, such as click chemistry, acid-amine coupling, Steglich esterification, etc.

In some embodiments, the conjugation between active agent and dendrimer is via a spacer that contain disulfide, ester, ether, thioester, carbamate, carbonate, hydrazine, ether, or amide linkages, or a combination thereof. In some embodiments, the conjugation between active agent and dendrimer is via an appropriate spacer that contain an ester linkage or an amide linkage between the agent and the dendrimer depending on the desired release kinetics of the agent.

The spacer can be chosen from among a class of compounds terminating in sulfhydryl, thiopyridine, succinimidyl, maleimide, vinylsulfone and carbonate group. The spacer can include thiopyridine terminated compounds such as dithiodipyridine, N-Succinimidyl 3-(2-pyridyldithio)-propionate (SPDP), Succinimidyl 6-(3-[2-pyridyldithio]-propionamido)hexanoate LC-SPDP or Sulfo-LC-SPDP. The spacer can also include peptides wherein the peptides are linear or cyclic essentially having sulfhydryl groups such as glutathione, homocysteine, cysteine and its derivatives, arg-gly-asp-cys (RGDC), cyclo(Arg-Gly-Asp-d-Phe-Cys) (c(RGDfC)), cyclo(Arg-Gly-Asp-D-Tyr-Cys), cyclo(Arg-Ala-Asp-d-Tyr-Cys). The spacer can be a mercapto acid derivative such as 3 mercapto propionic acid, mercapto acetic acid, 4 mercapto butyric acid, thiolan-2-one. 6 mercaptohexanoic acid, 5 mercapto valeric acid and other mercapto derivatives such as 2 mercaptoethanol and 2 mercaptoethylamine. The spacer can be thiosalicylic acid and its derivatives, (4-succinimidyloxycarbonyl-methyl-alpha-2-pyridylthio)toluene, (3-[2-pyridithio]propionyl hydrazide, The spacer can have maleimide terminations wherein the spacer includes polymer or small chemical entity such as bis-maleimido diethylene glycol and bis-maleimido triethylene glycol, Bis-Maleimidoethane, bismaleimidohexane. The spacer can include vinylsulfone such as 1,6-Hexane-bis-vinylsulfone. The spacer can include thioglycosides such as thioglucose. The spacer can be reduced proteins such as bovine serum albumin and human serum albumin, any thiol terminated compound capable of forming disulfide bonds. The spacer can include polyethylene glycol having maleimide, succinimidyl, and/or thiol terminations.

### D. Dendrimer-Agent Conjugates

Dendrimer-active agent conjugates can be formed of cannabinoids and/or cannabinoid derivatives covalently conjugated or non-covalently attached to a dendrimer, a dendritic polymer, or a hyperbranched polymer. Methods for conjugation of one or more active agents to a dendrimer are known, such as those described in U.S. Published 2011/0034422, 2012/0003155, and 2013/0136697. Covalent conjugates are preferred, but ionic complexes (cation-anion complexes) may also be used.

In some embodiments, one or more active agents are covalently conjugated to one or more terminal groups of the dendrimer such as terminal hydroxyl groups. In some embodiments, dendrimer conjugates include one or more active agents conjugated to the dendrimer via one or more spacers. The spacer between a dendrimer and an active agent can be designed to provide a releasable or non-releasable form of the dendrimer conjugate in vivo. For example, the spacer can be cleavable or contain a chemical linkage that is cleavable, for example, by exposure to the intracellular compartments of target neural and/or glial cells or upon binding to the receptor on the surface or in the interior of the target neural and/or glial cells in vivo. Examples of cleavable linkages that can be used in a spacer of the dendrimer-active agent conjugates include, esterase sensitive ester bond, glutathione sensitive disulfide bond, phosphatase-sensitive phosphodiester bond, oligopeptide such as triglycyl peptide linker capable of lysosomal release, acid cleavable hydrazine linkage etc. In some embodiments, the spacer between a dendrimer and active agents can provide desired and effective release kinetics in vivo. In some embodiments, the spacer between the dendrimer and the active agent can be non-cleavable or contain a chemical linkage that is non-cleavable, such as amide, ether, and amino alkyl linkages.

Generally, the spacer between the dendrimer and active agent has a length sufficient for the active agent conjugated thereto to reach and bind to the target receptor on the surface and/or inside of the target cell. For example, the spacer between the dendrimer and active agent has a length in a range from 50 Da to 2000 Da, depending on the release kinetics desired, and the receptor binding flexibility desired. The length of the spacer can vary, depending on the location of the target receptor (for example, on the cell surface, in the cytoplasm of the cell, or in an intercellular compartment of the cell) and/or density of the receptor when located on the cell surface.

The dendrimer can be a generation 2, generation 3, generation 4, generation 5, generation 6, and up to generation 10. In some embodiments, the dendrimer is conjugated to one or more active agents via spacers containing cleavable (ester, disulfide, phosphodiester, triglycyl peptide, and hydrazine) or non-cleavable (amide, ether, and amino alkyl) linkages.

The density of active agents covalently conjugated to or non-covalently attached to the dendrimer can be adjusted based on the specific cannabinoids or cannabinoid derivatives being delivered, the target receptors, the target neural and/or glial cells, the location of the target neural and/or glial cells, etc. For example, a plurality of active agents conjugated to the dendrimer are on the periphery of the dendrimer and the surface density of the active agent is at least 1 active agent/nm² (number of active agent conjugated/surface area in nm²), preferably 3-10. For example, in some embodiments, the surface density of active agent per nm² is more than 0.5, 1, 2, 3, 4, 5, 6, 7, 8, 9, 10, such as at least 10, 15, 20, 25, 30, 35, 40, 45, 50, or more than 50. In some embodiments, the volumetric density of active agent is between about 1 and about 50 groups/nm³, between about 5 and about 30 groups/nm³, or between about 10 and about 20 groups/nm³.

Typically, the dendrimer-active agent conjugates have a hydrodynamic volume in the nanometer range. For example, in some embodiments, the glucose dendrimer-active agent conjugates including one or more cannabinoids or cannabinoid derivatives conjugated to the dendrimer have a diameter of about 2 nm to about 100 nm, or more than 100 nm, up to 500 nm, depending upon the generation of dendrimer, the chemical composition and amount of active agent conjugated thereto. In some embodiments, a dendrimer-active agent conjugate including one or more cannabinoids or cannabinoid derivatives conjugated to the dendrimer has a diameter effective to penetrate brain tissue and to retain on the surface and/or in target neural and/or glial cells for a period of time sufficient for the active agent to bind to the targeted receptors on the surface and/or in the target neural and/or glial cells. In some embodiments, a dendrimer-active agent conjugate including one or more cannabinoids or cannabinoid derivatives conjugated to the dendrimer has a diameter effective to remain in the peripheral circulation and to retain on the surface and/or in target neural and/or glial cells for a period of time sufficient for the active agent to bind to the targeted receptors on the surface and/or in the target neural and/or glial cells.

The dendrimer-active agent conjugates can be neural, have a positive charge or a negative charge. In some embodiments, the dendrimer-active agent conjugates are neutral. The presence of cannabinoids or cannabinoid derivatives can affect the surface charge of the dendrimer-active agent conjugates. In some embodiments, the surface charge of the dendrimer conjugated to cannabinoids or cannabinoid derivatives is between −100 mV and 100 mV, between −50 mV and 50 mV, between −25 mV and 25 mV, between −20 mV and 20 mV, between −10 mV and 10 mV, between −10 mV and 5 mV, between −5 mV and 5 mV, or between −2 mV and 2 mV. The range above is inclusive of all values from −100 mV to 100 mV. In preferred embodiments, the surface charge of the dendrimer-active agent conjugates is neutral or near-neutral, i.e., from about −10 mV to about 10 mV, inclusive.

An exemplary dendrimer-active agent conjugate is represented by Formula (I). The dendrimer of the exemplary conjugate contains surface hydroxyl groups, wherein one or more of the surface hydroxyl groups are conjugate to one or more active agents via one or more spacers as shown in Formula (I) below.

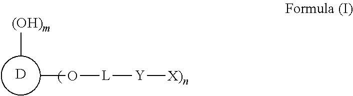

wherein D can be a generation 1 to generation 10 or generation 2 to generation 10 dendrimer, such as any one of those described above, for example, PAMAM (such as hydroxyl-terminated PAMAM dendrimer) or a glucose-based dendrimer; each occurrence of L can be any suitable chemical moiety appropriate for providing tailored drug release and receptor binding, preferably containing a triazole moiety; Y can be a bond or a linkage selected from secondary amides (—CONH—), tertiary amides (—CONR—), sulfonamide (—S(O)₂—NR—), secondary carbamates (—OCONH—; —NHCOO—), tertiary carbamates (—OCONR—; —NRCOO—), carbonate (—O—C(O)—O—), ureas (—NHCONH—; —NRCONH—; —NHCONR—, —NRCONR—), carbinols (—CHOH—, —CROH—), disulfide groups, phosphodiester group

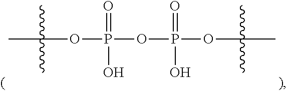 [SMILES: *OP(=O)(O)OP(=O)(O)O*]

hydrazino group, hydrazones, hydrazides, ester (—C(O)—O—), ether (—O—), and oligopeptide (e.g., triglycyl peptide), wherein R is an alkyl group, an aryl group, or a heterocyclic group; each occurrence of X can be a cannabinoids or cannabinoid derivatives, wherein a functional group of X (such as an amino group including primary amino, secondary amino, or tertiary amino group; a carboxylic group; or a hydroxyl group) forms a portion of linkage Y; n can be an integer from 1 to 100; and m can be an integer from 16 to 4096. The dendrimer can be PAMAM or a glucose dendrimer, which is 100% hydroxyl. m and n depend on the size of the dendrimer D, n should be such that the weight percent of the drug in the total conjugate is ‘5-20%. This range is also appropriate for binding and internalization.

The oxygen atom shown in Formula (I) is from the surface functional group of the dendrimer, such as a surface hydroxyl group, where the surface hydroxyl group may or may not be part of a terminal sugar moiety/molecule (e.g., glucose). Although not illustrated in Formula (I), one or more hydroxyl groups of the dendrimer that are not conjugated to active agents may be modified with one or more carbohydrates and/or polyalkylene glycols, such as PEG.

When administered to a subject in need thereof, the cannabinoid and/or cannabinoid derivative X of Formula (I) can bind to a target receptor on the surface of the target cell or inside the target cell. In some embodiments, when the cannabinoid and/or cannabinoid derivative X binds to the target receptor, the agent X remains conjugated to the dendrimer. In these embodiments, following binding, the agent X may be released from the dendrimer or remain conjugated to the dendrimer as an intact dendrimer-active agent conjugate. In some embodiments, the cannabinoid and/or cannabinoid derivative X is released from the dendrimer at close proximity to the target receptor and then binds to the target receptor.

In some embodiments, each occurrence of L can be represented by -A′-L1-B′-L2-, wherein A′ can be a carbonyl (—C(O)—) or a bond (including single, double, and triple bonds, for example a single bond); B′ can be a bond (including single, double, and triple bonds, for example a single bond), an amide, an ester, an ether, a thiol, a dithiol, an aryl, a heteroaryl, a polyaryl, a heteropolyaryl, or a heterocyclic; and L1 and L2 can be independently a bond, an alkylene, a heteroalkylene, an aryl, an aralkyl, an ether, a polyether, a thiol, a dithiol, a thiolether, a polythioether, an oligopeptide, a polypeptide, an oligo(alkylene glycol), or a polyalkylene glycol, or L1 and L2 can be independently composed of a combination of these groups, such as a combination of alkylene and polyether, a combination of alkylene and thiol or dithiol, a combination of alkylene and oligopeptide, a combination of alkylene, polyether, and thiol or dithiol, or a combination of polyether and thiol or dithiol. In some forms, L1-B′-L2-together form a chemical moiety selected from an -alkylene-triazole-di(alkylene glycol)-, a -di(alkylene glycol)-triazole-alkylene-, -alkylene-triazole-oligo(alkylene glycol)-, an -oligo(alkylene glycol)-triazole-alkylene-, an -alkylene-triazole-poly(alkylene glycol)-, -poly(alkylene glycol)-triazole-alkylene-, an -alkylene-triazole-ether-, an -alkylene-triazole-alkylene-, an -alkylene-amide-alkylene-, and combinations thereof.

In some embodiments, B′ can be a bond (including single, double, and triple bonds, for example a single bond), an amide group, or a heterocyclic group, such as a triazole group.

In some embodiments, L1 can be a bond; an alkylene, such as a C₁-C₁₀ alkylene, a C₁-C₈ alkylene, a C₁-C₆ alkylene, a C₁-C₅ alkylene, a C₁-C₄ alkylene, or a C₁-C₃ alkylene; or an oligo- or poly-(alkylene glycol), such as

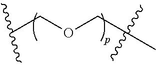

where p is an integer from 1 to 20, from 1 to 18, from 1 to 16, from 1 to 14, from 1 to 12, from 1 to 10, from 1 to 8, from 1 to 6, from 1 to 5, from 1 to 4, from 1 to 3, or 1 or 2.

In some embodiments, L2 can be a bond; an alkylene, such as a C₁-C₁₀ alkylene, a C₁-C₈ alkylene, a C₁-C₆ alkylene, a C₁-C₅ alkylene, a C₁-C₄ alkylene, or a C₁-C₃ alkylene; an oligo- or poly-(alkylene glycol), such as

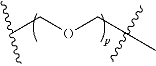

where p is an integer from 1 to 20, from 1 to 18, from 1 to 16, from 1 to 14, from 1 to 12, from 1 to 10, from 1 to 8, from 1 to 6, from 1 to 5, from 1 to 4, from 1 to 3, or 1 or 2; an oligo- or poly-peptide, such as a triglycyl peptide; a thiol; or a dithiol; or L2 is composed of a combination of two or more of alkylene, oligo- or poly-(alkylene glycol), oligo- or poly-peptide, thiols, and dithiols. For example, L2 is represented by

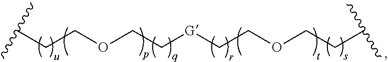

where p, q, r, s, t, and u are independently an integer from 0 to 10, from 0 to 8, from 0 to 6, from 0 to 5, from 0 to 4, from 0 to 3, or from 0 to 2, such as 0, 1, or 2; and G′ is a thiol, a dithiol, an oligo-peptide such as a triglycyl peptide, or a poly-peptide.

In some embodiments, Y is a linkage that is minimally cleavable in vivo. In some embodiments, Y is a linkage that is cleavable in vivo. In some embodiments, Y is an amide (—CONH—), an ester (—C(O)—O—), an ether (—O—), a phosphodiester, or a disulfide group.

In some embodiments, L and Y are both a single bond, and D is directly conjugated to X (an active agent or analog thereof) via an ether linkage.

In some embodiments, D is a generation 2 PAMAM dendrimer, a generation 3 PAMAM dendrimer, a generation 4 PAMAM dendrimer, a generation 5 PAMAM dendrimer, a generation 6 PAMAM dendrimer, a generation 1 glucose dendrimer, a generation 2 glucose dendrimer, a generation 3 glucose dendrimer, a generation 4 glucose dendrimer, a generation 5 glucose dendrimer, or a generation 6 glucose dendrimer.

More specific exemplary dendrimer-active agent conjugates are shown in the Examples below.

### E. Exemplary Cannabinoid-Dendrimer Conjugates

In preferred embodiments, the dendrimer is conjugated to cannabidiol (CBD) as shown in Structures A and B below, wherein n is an integer from 1 to 10, such as 1,2, 3, 4, 5, 6, 7, 8, 9, or 10.

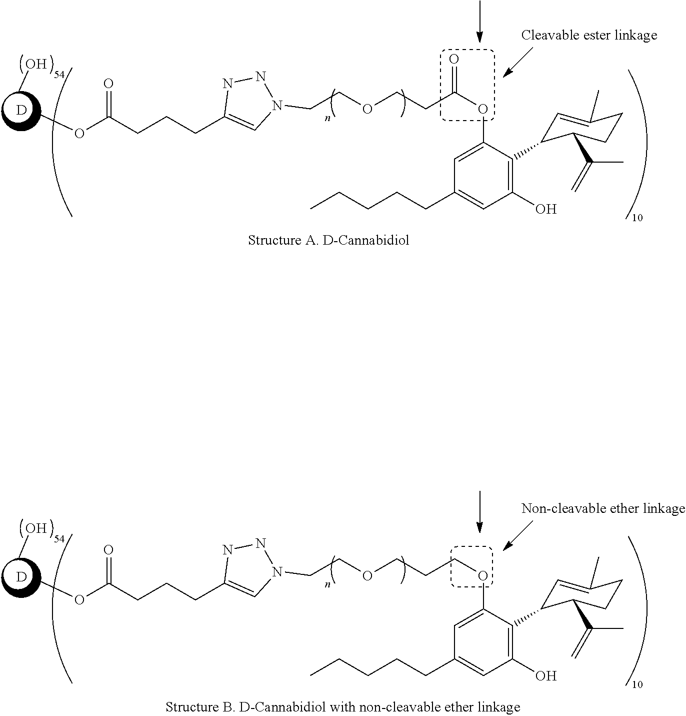

In other preferred embodiments, the dendrimer is conjugated to HU-308 as shown in Structures C-E below, wherein n is an integer from 1 to 10, such as 1, 2, 3, 4, 5, 6, 7, 8, 9, or 10.

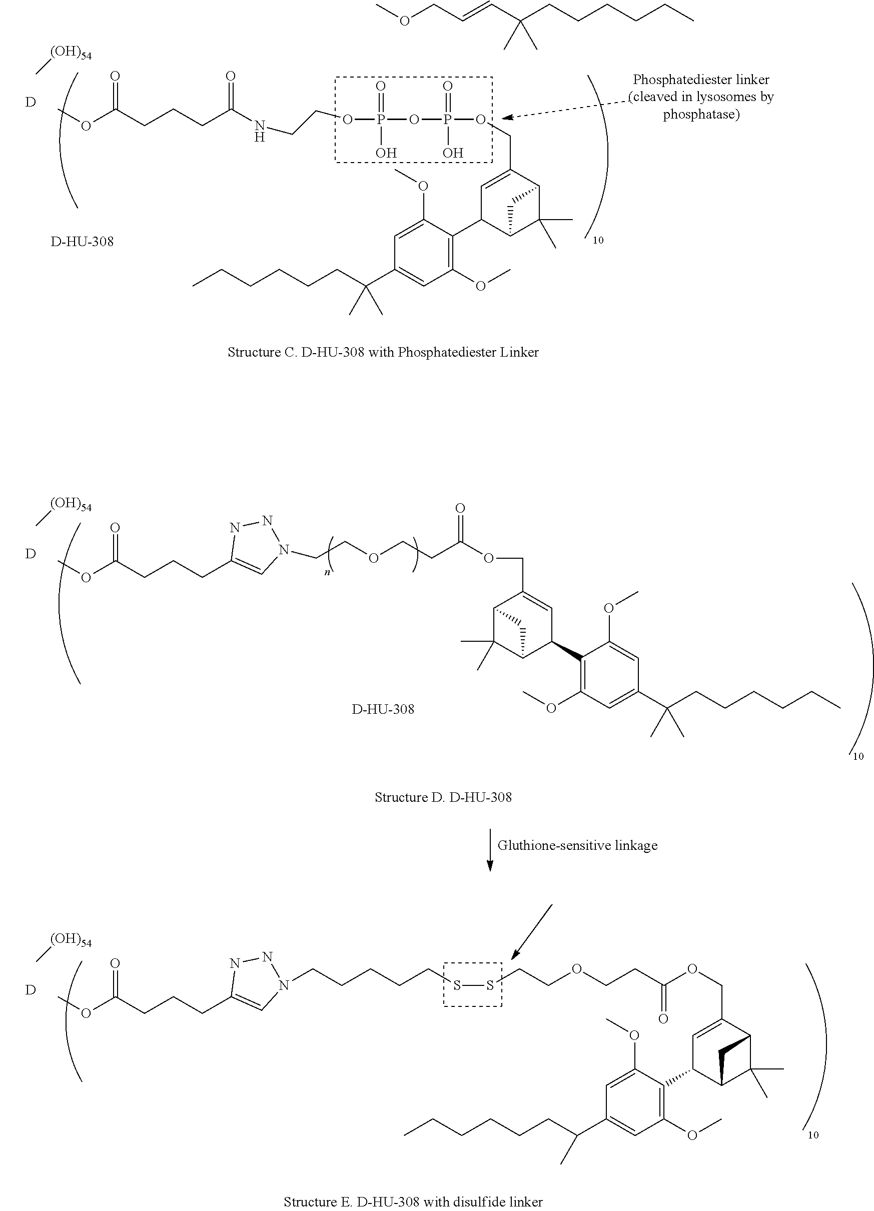

In other preferred embodiments, the dendrimer is conjugated to THC as shown in Structures F-G below.

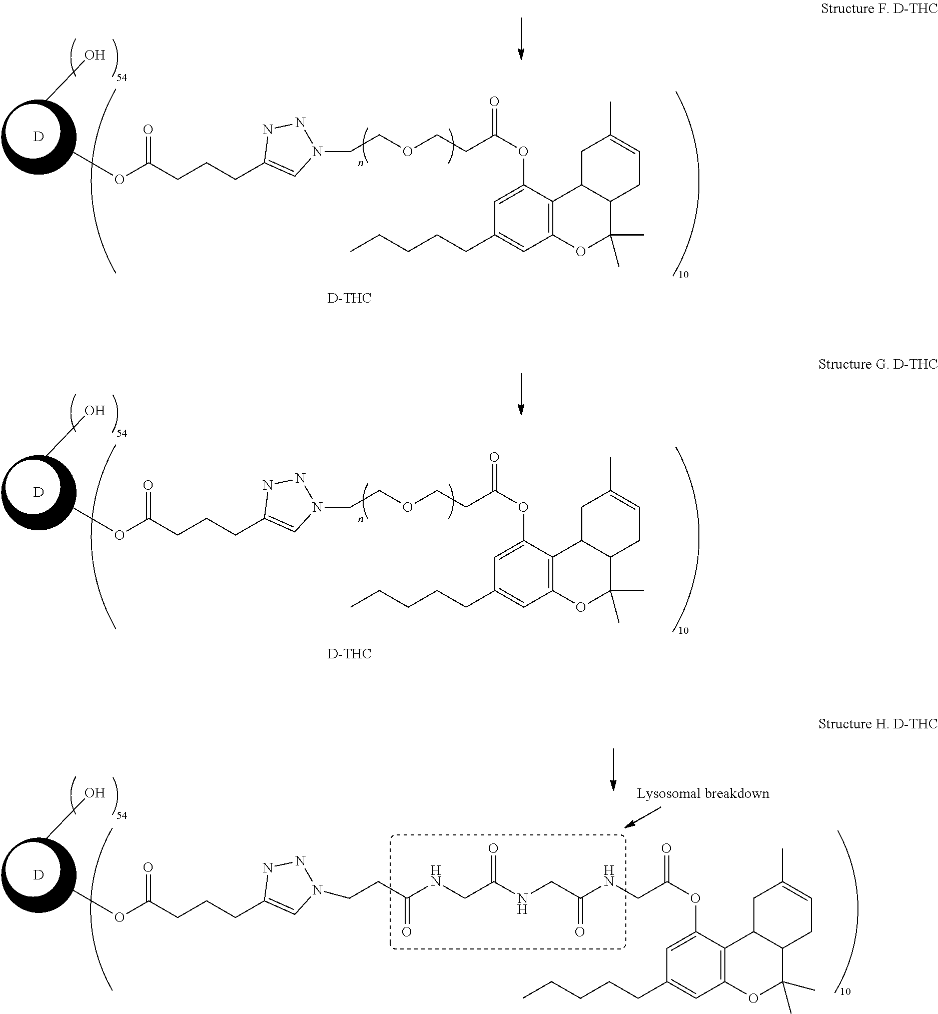

In other preferred embodiments, the dendrimer is conjugated to AEA as shown in Structures I-J below, wherein n is an integer from 1 to 10, such as 1, 2, 3, 4, 5, 6, 7, 8, 9, or 10.

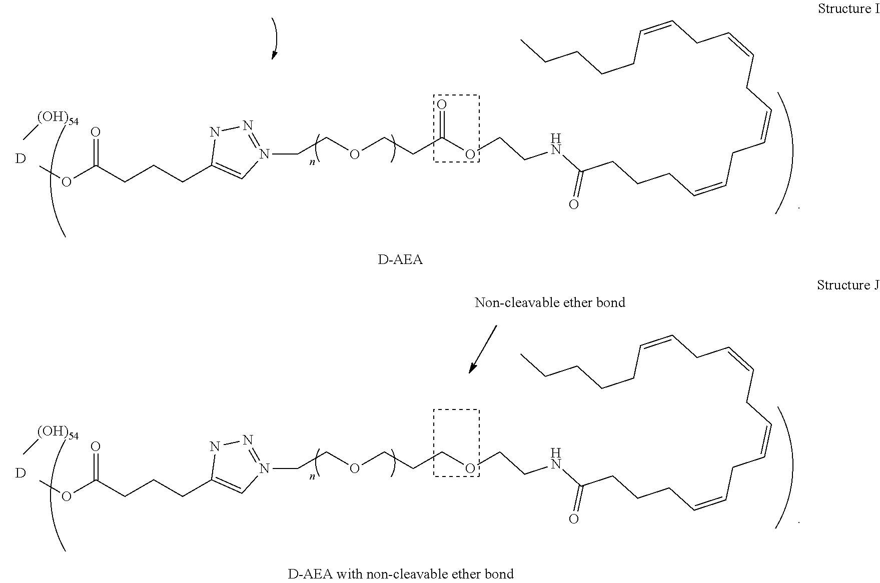

In another preferred embodiment, the dendrimer is conjugated to 2 AG as shown in Structure K below, wherein n is an integer from 1 to 10, such as 1, 2, 3, 4, 5, 6, 7, 8, 9, or 10.

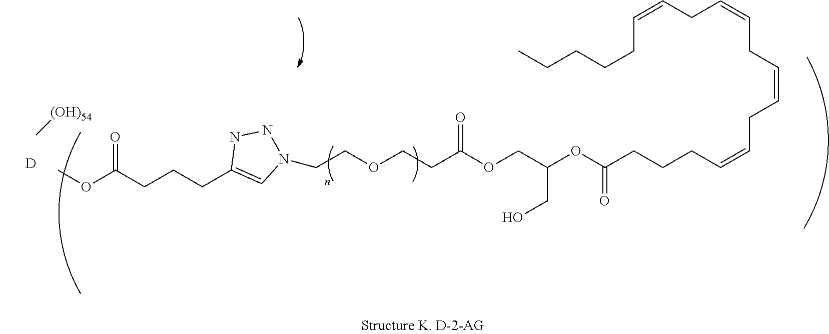

### III. Methods of Making Dendrimer Conjugates

### A. Methods of Making Dendrimers

Dendrimers, in particular, glucose dendrimers, can be prepared via a variety of chemical reaction steps. Dendrimers are usually synthesized according to methods allowing controlling their structure at every stage of construction. The dendritic structures are mostly synthesized by two main different approaches: divergent or convergent.

In some embodiments, dendrimers are prepared using divergent methods, in which the dendrimer is assembled from a multifunctional core, which is extended outward by a series of reactions, commonly a Michael reaction. The strategy involves the coupling of monomeric molecules that possesses reactive and protective groups with the multifunctional core moiety, which leads to stepwise addition of generations around the core followed by removal of protecting groups. For example, dendrimers are first synthesized by coupling AB4 peracetylated β-D glucose-PEG4-azide monomers to hexapropargylated core. In another example, PAMAM-NH₂ dendrimers are first synthesized by coupling N-(2-aminoethyl) acryl amide monomers to an ammonia core.

In other embodiments, dendrimers are prepared using convergent methods, in which dendrimers are built from small molecules that end up at the surface of the sphere, and reactions proceed inward, building inward, and are eventually attached to a core.

Many other synthetic pathways exist for the preparation of dendrimers, such as the orthogonal approach, accelerated approaches, the Double-stage convergent method or the hypercore approach, the hypermonomer method or the branched monomer approach, the Double exponential method; the Orthogonal coupling method or the two-step approach, the two monomers approach, AB₂-CD₂ approach.

In some embodiments, the core of the dendrimer, one or more branching units, one or more spacers, and/or one or more surface functional groups can be modified to allow conjugation to further functional groups (branching units, spacers, surface functional groups, etc.), monomers, and/or agents via click chemistry, employing one or more Copper-Assisted Azide-Alkyne Cycloaddition (CuAAC), Diels-Alder reaction, thiol-ene and thiol-yne reactions, and azide-alkyne reactions (Arseneault M et al., Molecules 2015 May 20; 20(5):9263-94). In some embodiments, pre-made dendrons are clicked onto high-density hydroxyl polymers. ‘Click chemistry’ involves, for example, the coupling of two different moieties (e.g., a core group and a branching unit; or a branching unit and a surface functional group) via a 1,3-dipolar cycloaddition reaction between an alkyne moiety (or equivalent thereof) on the surface of the first moiety and an azide moiety (e.g., present on a triazine composition or equivalent thereof), or any active end group such as, for example, a primary amine end group, a hydroxyl end group, a carboxylic acid end group, a thiol end group, etc.) on the second moiety. In some embodiments, one or more hydroxyl groups on the surface of the dendrimer (hydroxyl-terminated PAMAM dendrimer or glucose dendrimer) are modified to contain an alkyl group and a drug is modified to contain an azide group. Alternatively, one or more hydroxyl groups on the surface of the dendrimer (hydroxyl-terminated PAMAM dendrimer or glucose dendrimer) are modified to contain an azide group and a drug is modified to contain an alkyne group. The azide and alkyne are then reacted via a 1,3-dipolor cycloaddition reaction to form a triazole moiety.

In some embodiments, dendrimer synthesis relies upon one or more reactions such as thiol-ene click reactions, thiol-yne click reactions, CuAAC, Diels-Alder click reactions, azide-alkyne click reactions, Michael Addition, epoxy opening, esterification, silane chemistry, and a combination thereof.

In some embodiments, methods involve one or more protection and deprotection steps of the function groups (e.g., hydroxyl groups) on the central core, branching units, and/or therapeutic, prophylactic or diagnostic agents to facilitate addition of branching units to generate desired dendrimer molecules, or addition of therapeutic, prophylactic or diagnostic agents to generate desired dendrimer conjugates. In the case of hydroxyl groups, they may be protected by formation of an ether, an ester, or an acetal. Other exemplary protection groups include Boc and Fmoc.

Any existing dendritic platforms can be used to make dendrimers of desired functionalities, i.e., with a high-density of surface hydroxyl groups by conjugating high-hydroxyl containing moieties such as 1-thio-glycerol or pentaerythritol. Exemplary dendritic platforms such as polyamidoamine (PAMAM), poly (propylene imine) (PPI), poly-L-lysine, melamine, poly (etherhydroxylamine) (PEHAM), poly (esteramine) (PEA) and polyglycerol can be synthesized and explored.

Dendrimers also can be prepared by combining two or more dendrons. Dendrons are wedge-shaped sections of dendrimers with reactive focal point functional groups. Many dendron scaffolds are commercially available. They come in 1, 2, 3, 4, 5, and 6th generations with, respectively, 2, 4, 8, 16, 32, and 64 reactive groups. In certain embodiments, one type of agents is linked to one type of dendron and a different type of agent is linked to another type of dendron. The two dendrons are then connected to form a dendrimer. The two dendrons can be linked via click chemistry i.e., a 1,3-dipolar cycloaddition reaction between an azide moiety on one dendron and alkyne moiety on another to form a triazole linker.

Exemplary methods of making dendrimers are described in detail in Intemational Patent Publication Nos. WO2009/046446, WO2015168347, WO2016025745, WO2016025741, WO2019094952, and U.S. Pat. No. 8,889,101.

### 1. Methods of Making Glucose Dendrimers

In some embodiments, glucose-based dendrimers are prepared using divergent methods, in which the dendrimer is assembled from a multifunctional core, which is extended outward by a series of reactions. The strategy involves the coupling of monomeric molecules that possesses reactive and protective groups with the multifunctional core moiety, which leads to stepwise addition of generations around the core followed by removal of protecting groups.

In some embodiments, glucose dendrimers are synthesized by coupling AB₄ peracetylated β-D glucose-PEG4-azide monomers to hexapropargylated core. In preferred embodiments, the hypercore is prepared from dipentaerythritol, for example by performing propargylation of dipentaerythritol to achieve the hexa-propargylated core. An exemplary scheme for preparing such a glucose dendrimer is shown by Scheme I.

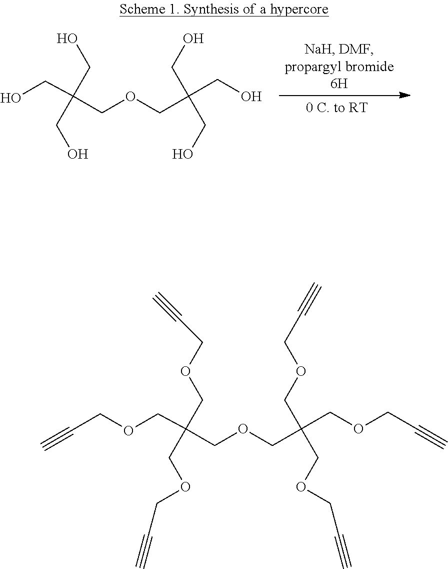 [SMILES: OCC(CO)(CO)COCC(CO)(CO)CO ; C#CCOCC(COCC#C)(COCC#C)COCC(COCC#C)(COCC#C)COCC#C]

In some embodiments, the branching units are hypermonomers i.e., AB₁ building blocks. Exemplary hypermonomers include AB₃, AB₄, AB₅, AB₆, AB₇, AB₈ building blocks. Hypermonomer strategy drastically increases the number of available end groups. An exemplary hypermonomer is AB₄ orthogonal hypermonomer including one azide functional group and four allyl groups prepared from dipentaerythritol with five allyl groups reacted with mono tosylated triethylene glycol azide.

In some embodiments, the branching unit is polyethylene glycerol linear or branched e.g., as shown by Formula III. Other monomers include disaccharides and oligosaccharides, as well as saccharides such as fructose, lactose, and sucrose.

a. Synthesis of AB₄ Building Block

Some exemplary synthesis methods of hypermonomer AB₄ are described below. In some embodiments, the hypermonomer AB₄ is based on glucose molecules. In preferred embodiments, the hypermonomer AB₄ is conjugated to a polyethylene glycerol, for example, tetraethylene glycol (PEG4). In one embodiment, the hypermonomer AB₄ is peracetylated β-D-Glucopyranoside tetraethylene glycol azide.

In some embodiments, the synthesis of glucose-OAc-TEG-OTs involves the following steps: a solution of peracetylated β-D-glucopyranoside (10 g, 25.6 mmol) was dissolved in 50 mL of anhydrous dichloromethane (DCM) followed by addition of 2-(2-(2-(2-hydroxyethoxy)ethoxy)ethoxy)ethyl 4-methylbenzenesulfonate (6.2 g, 17.9 mmol) and the reaction mixture was cooled to 0° C. Boron trifluoride diethyl etherate (2.5 eq.) was added and the reaction was allowed to come to room temperature. The reaction was monitored with the help of TLC and quenched after 5 hrs by the addition of saturated sodium bicarbonate solution at 0° C. After 10 minutes of stirring, DCM (300 mL) was added and the organic layer was washed with saturated sodium bicarbonate solution 3 times until the effervescence was quenched. The reaction mixture was dried over sodium sulfate, filtered, and evaporated under reduced pressure. The crude product was purified by combiflash chromatography using ethyl acetate/hexanes (70:30) mixture as eluents. The desired compound was achieved in 60% yield. Structure of glucose-OAc-TEG-OTs is shown below:

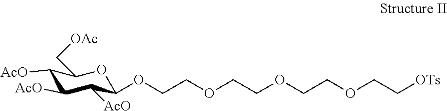

In some embodiments, the synthesis of glucose-OAc-TEG-N₃ involves the following steps: a solution of glucose-OAc-TEG-OTs (6 g, 8.8 mmoles) is dissolved in 40 mL of anhydrous DMF followed by the addition of sodium azide (2 eq) and the reaction mixture is heated to 50° C. for overnight. Upon completion, the reaction mixture is filtered and DMF is evaporated. Once dried, the crude reaction mixture is passed through combiflash using ethyl acetate:hexane (70:30) as eluent. Structure of glucose-OAc-TEG-N₃ is shown below:

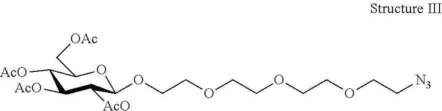

In some embodiments, the synthesis of glucose-OH-TEG-N₃ involves the following steps: the peracetylated β-D-Glucopyranoside tetraethylene glycol azide is dissolved in anhydrous methanol and sodium methoxide is added to adjust the pH around 8.5-9. The reaction is stirred overnight at room temperature, then diluted with methanol and pH is adjusted with Amberlist IR-120+ around 6-7. The reaction mixture is separated by filtration and the solvent removed by rotary evaporation. Structure of glucose-OH-TEG-N₃ is shown below.

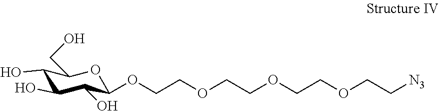

b. Synthesis of Glucose Dendrimers

In some embodiments, glucose dendrimers are synthesized by coupling AB₄ peracetylated β-D glucose-PEG4-azide monomers to hexapropargylated core. In preferred embodiments, the hexapropargylated core is linked to AB₄ β-D-glucose-PEG4-azide building block (2) via click reaction to obtain generation 1 dendrimer.

In some embodiments, generation one dendrimer D1-Glu6-OAc24 is prepared according to the following: Hexapropargylated compound (0.5 g, 1 mmoles) and an azido derivative ((4.1 g, 7.4 mmoles) 1.2 eq. per acetylene) are suspended in a 1:1 mixture of DMF and water in a 20 mL microwave vial equipped with a magnetic stir bar. CuSO₄·5H₂O (5 mol %/acetylene, 75 mg) and sodium ascorbate (5 mol %/acetylene, 60 mg) dissolved in the minimum amount of water are added. The reaction is irradiated in a microwave at 50° C. for 6 h. The reaction mixture is dialyzed against DMF followed by water dialysis containing EDTA. The EDTA is further removed by extensive water dialysis. The product is lyophilized to obtain D1-Glu6-OAc24. Structure of D1-Glu6-OAc24 is shown below.

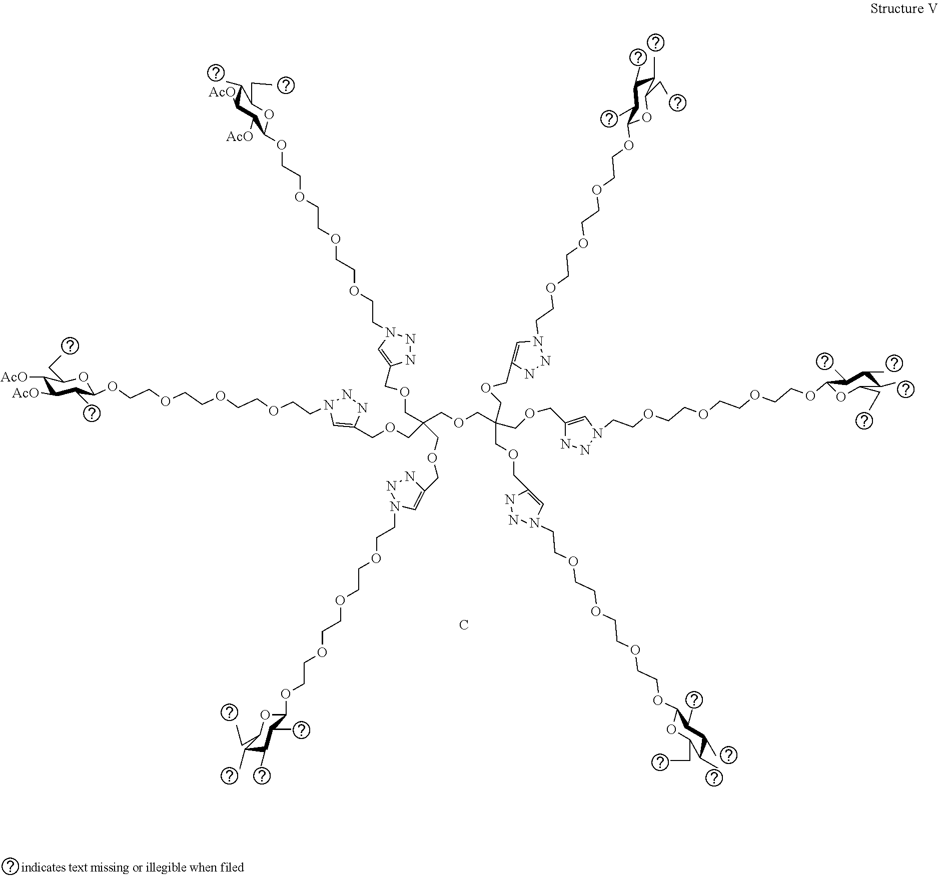

In some embodiments, generation one dendrimer D1-Glu₆-OH24 is prepared according to the following: the peracetylated generation 1 glucose dendrimer (1 g, 0.26 mmoles) is dissolved in anhydrous methanol and sodium methoxide is added to adjust the pH to around 8.5-9. The reaction is stirred overnight at room temperature, then diluted with methanol and pH is adjusted with AMBERLIST® IR-120+ around 6-7. The reaction mixture is separated by filtration and the solvent removed by rotary evaporation, followed by water dialysis. Structure of generation one glucose dendrimer, D1-Glu6-OH24, is shown below.

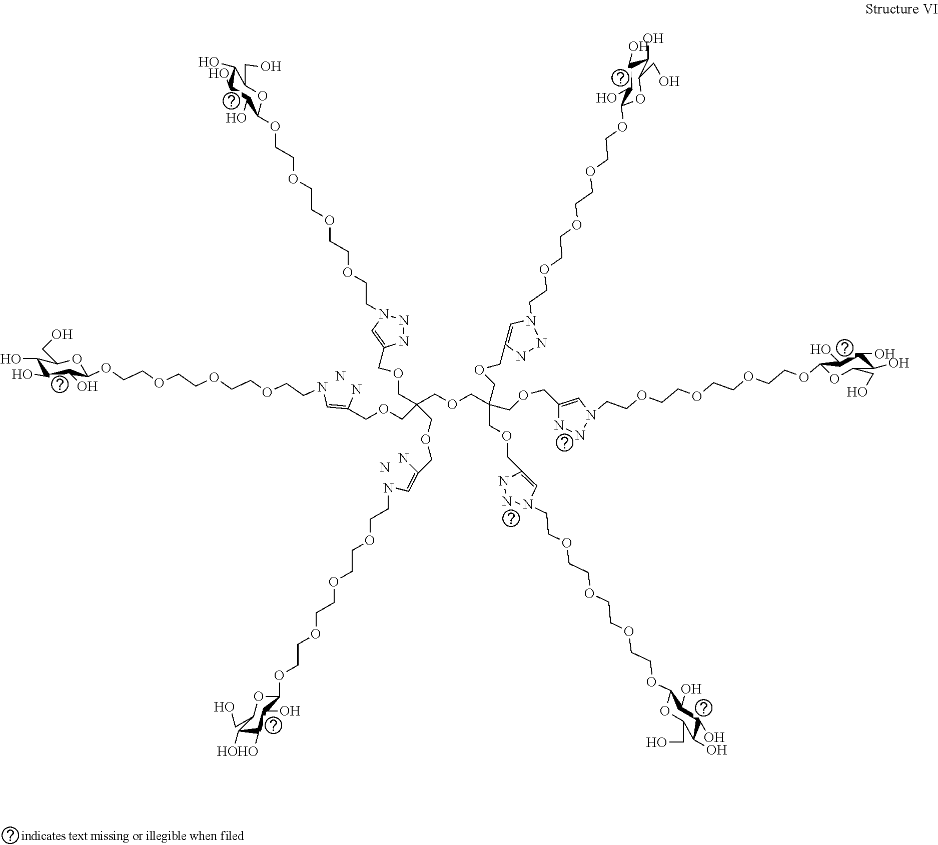

In some embodiments, generation one glucose dendrimer D1-Glu6-OH24 is propargylated to provide D1-Acetylene24 according to the following: D1-GLu6-OH24 (2 g, 0.721 mmol) was dissolved in anhydrous dimethylformamide (DMF, 50 mL) by sonication. Sodium hydride [60% dispersion in mineral oil](951 mg, 39.65 mmol) is slowly added in portions at 0° C. to the solution with stirring. The solution is stirred for an addition 15 minutes at 0° C. This is followed by the addition of propargyl bromide (3.85 mL, 34.608 mmol, 80% w/w solution in toluene) at 0° C. and the stirring is continued at room temperature for another 6 h. The reaction mixture is quenched with ice and water, filtered, and dialyzed against DMF, followed by the water dialysis to afford D1-acetylene24. Structure of D1-acetylene24 is shown below.

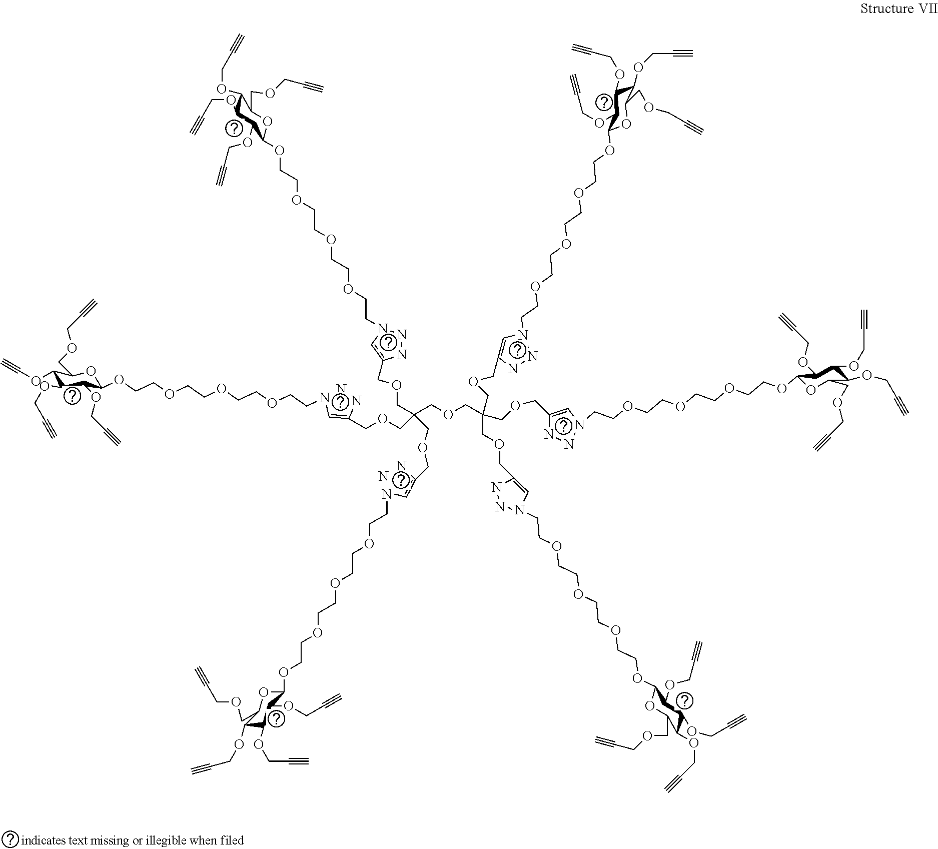

In some embodiments, generation one dendrimer D1-acetylene24 is further reacted with AB₄ β-D-glucose-PEG4-azide to provide generation 2 dendrimer with 24 glucose molecules containing 96 surface hydroxyl groups.

An exemplary generation two dendrimer D2-Glu24-OAc96 is prepared according to the following: D1-acetylene dendrimer24 (0.5 g, 0.13 mmoles) and glucose-OAc-TEG-azide (2.2 g, 4 mmoles) are suspended in a 1:1 mixture of DMF and water in a 20 mL microwave vial equipped with a magnetic stir bar. To this CuSO₄·5H₂O (5 mol %/acetylene, 5 mg) and sodium ascorbate (5 mol %/acetylene, 10 mg) dissolved in the minimum amount of water are added. The reaction is irradiated in a microwave at 50° C. for 8 h. Upon completion, the reaction mixture is dialyzed against DMF followed by water dialysis containing EDTA. The EDTA is further removed by extensive water dialysis. The product is lyophilized to obtain D2-Glu24-OAc96.

In some embodiments, generation two dendrimer D2-Glu24-OH96 is prepared according to the following: the peracetylated generation 2 glucose dendrimer D2-Glu24-OH96 is dissolved in anhydrous methanol and sodium methoxide is added to adjust the pH around 8.5-9.0. The reaction is stirred overnight at room temperature, then diluted with methanol and pH is adjusted with AMBERLIST® IR-120+ around 6-7. The reaction mixture is filtered to remove the resin and the filtrate is evaporated by rotary evaporation followed by water dialysis to obtain the product as off-white solid.

Structure of generation two glucose dendrimer, D2-Glu24-OH96, is shown below.

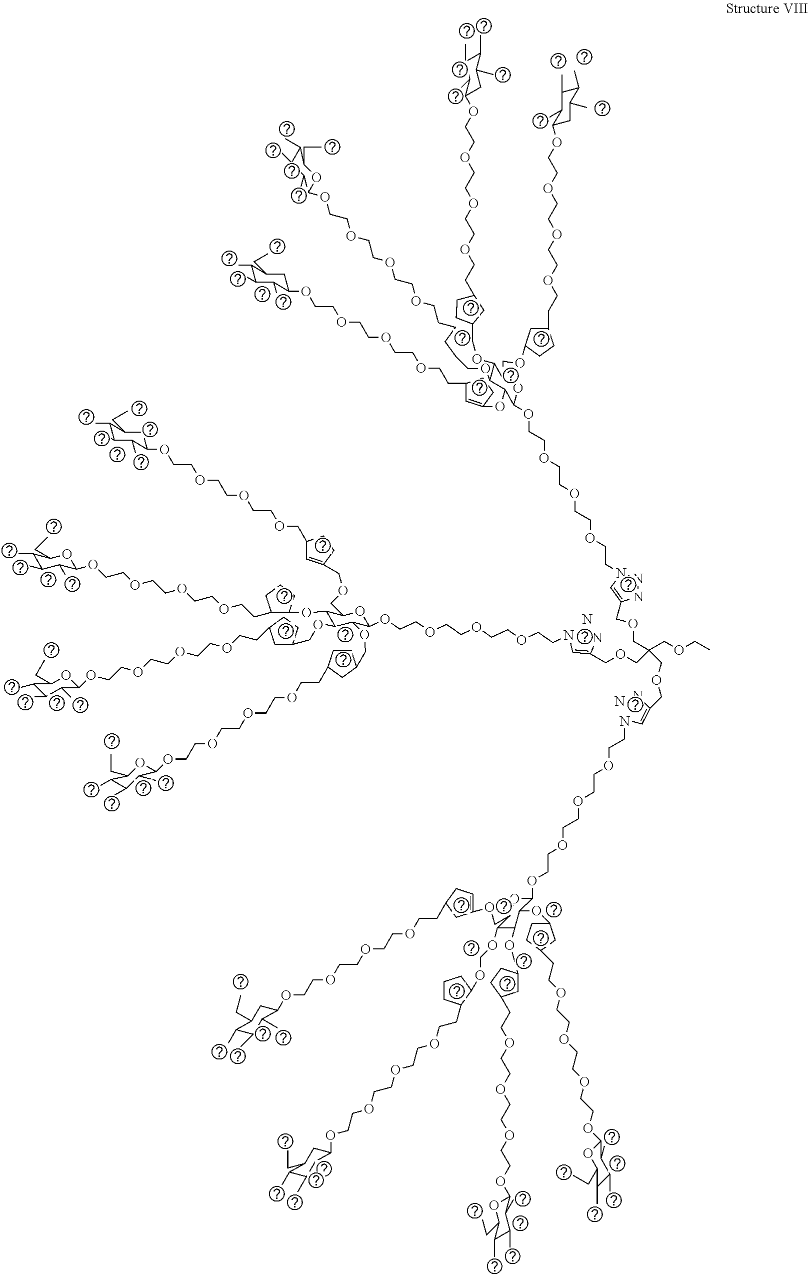

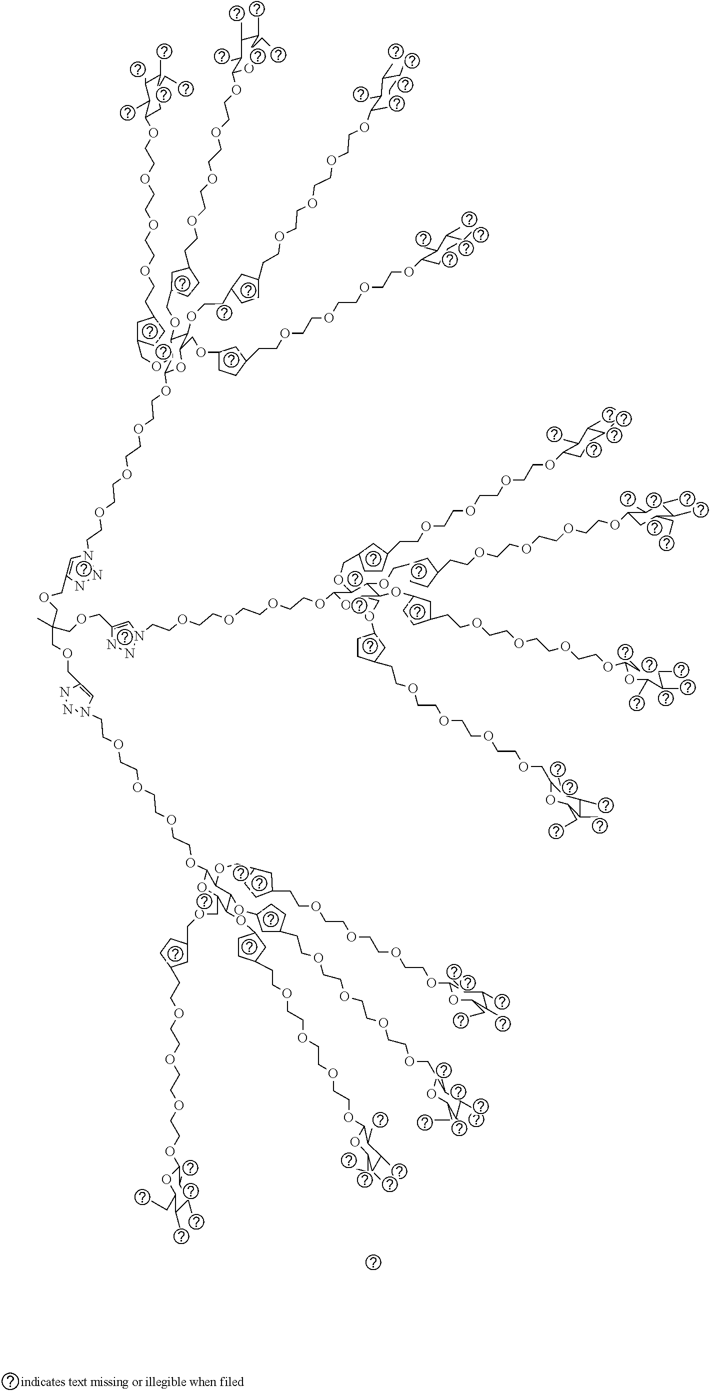

In some embodiments, generation two dendrimer D2-Glu24-OH96 is propargylated at one or more terminal hydroxyl groups suitable for further conjugation to one or more therapeutic, prophylactic or diagnostic agents. In some embodiments, one or more terminal hydroxyl groups of generation two dendrimer D2-Glu24-OH96 is propargylated according to the following: D2-Glu24-OH96 (5b) (200 mg, 0.016 mmol) is dissolved in anhydrous dimethylformamide (DMF, 10 mL) by sonication. To this stirring solution, sodium hydride [60% dispersion in mineral oil](22 mg, 0.934 mmol) is slowly added in portions at 0° C. The solution is additionally stirred for 15 minutes at 0° C. This is followed by the addition of propargyl bromide (18.0 μL, 80% w/w solution in toluene) at 0° C. and the stirring is continued at room temperature for another 6 h. The solvent is evaporated using V10 evaporator system and the crude product is purified by passing through PD10 SEPHADEX® G25 M column. The aqueous solution is lyophilized to afford the product as off-white solid.

In some embodiments, one or more fluorescent dyes such as infrared fluorescent Cy5 dyes are conjugated to generation two dendrimer D2-Glu24-OH96. In one embodiment, Cy5-D2-Glu24-OH96 (compound 7 of FIG. 1B) is prepared according to the following: Compound 6 (200 mg, 0.016 mmol) and Cy5 azide (20.7 mg, 0.02 mmol) are suspended in a 1:1 mixture of DMF and water in a 25 mL round bottom flask equipped with a magnetic stir bar. To this, CuSO₄·5H₂O (5 mol %/acetylene, 0.3 mg) and sodium ascorbate (10 mol %/acetylene, 0.5 mg) dissolved in the minimum amount of water are added. The reaction is stirred at room temperature for 24 h. Upon completion, the DMF is evaporated using V10 and the purification is performed using PD10 Sephadex G25 M column. The aqueous solution is lyophilized to afford the product as blue solid.

In some embodiments, the total hydroxyl groups for further conjugation to active agents including therapeutic and/or diagnostic agents are about 1-30, 2-20, or 5-10 out of total 96 available hydroxyl groups of the exemplary generation 2 dendrimer with 24 glucose molecules containing 96 surface hydroxyl groups.

### B. Methods of Making Dendrimer-Agent Conjugates

Methods for conjugating agents with dendrimers are generally known in the art and for example, as described in U.S. Published Application Nos. US 2011/0034422, US 2012/0003155, and US 2013/0136697.

In some embodiments, one or more agents are covalently attached to the dendrimers. In some embodiments, the agents are attached to the dendrimer via a spacer that is designed to be non-cleavable in vivo. In some embodiments, the agents are attached to the dendrimer via a spacer that is designed to be cleaved in vivo. For example, the spacer can be designed to be cleaved hydrolytically, enzymatically, or combinations thereof, so as to provide for the sustained release of the agents in vivo. In some embodiments, both the chemical structure of the spacer and its point of attachment to the agent, can be selected so that cleavage of the spacer releases either an agent, or a suitable prodrug thereof. The chemical structure of the spacer can also be selected in view of the desired release rate of the agents.

In some embodiments, the conjugation between the agent and dendrimer is via one or more of disulfide, ester, ether, phosphodiester, triglycyl peptide, hydrazine, amide, or amino alkyl linkages. In some embodiments, the conjugation between the agent and dendrimer is via an appropriate spacer that provides an ester bond or an amide bond between the agent and the dendrimer depending on the desired release kinetics of the agent. In some cases, an ester or disulfide bond is introduced for releasable form of agents. In other cases, an amide or amino alkyl bond is introduced for non-releasable form of agents.

Spacers generally contain one or more organic functional groups. Examples of suitable organic functional groups contained in the spacers include secondary amides (—CONH—), tertiary amides (—CONR—), sulfonamide (—S(O)₂—NR—), secondary carbamates (—OCONH—; —NHCOO—), tertiary carbamates (—OCONR—; —NRCOO—), carbonate (—O—C(O)—O—), ureas (—NHCONH—; —NRCONH—; —NHCONR—, —NRCONR—), carbinols (—CHOH—, —CROH—), disulfide groups, hydrazones, hydrazides, ethers (—O—), and esters (—COO—, —CH₂O₂C—, CHRO₂C—), wherein R is an alkyl group, an aryl group, or a heterocyclic group. In general, the identity of the one or more organic functional groups within the spacer is chosen in view of the desired release rate of the agents. In addition, the one or more organic functional groups can be selected to facilitate the covalent conjugation of the agents to the dendrimers. In some embodiments, the conjugation between the agent and dendrimer is via an appropriate spacer that provides a disulfide bridge between the agent and the dendrimer. In some embodiments, the dendrimer-active agent conjugates are capable of rapid release of the agent in vivo by thiol exchange reactions, under the reduced conditions found in body.

In certain embodiments, the spacer contains one or more of the organic functional groups described above in combination with a linking group. The linking group can be composed of any assembly of atoms, including oligomeric and polymeric chains; for example, the total number of atoms in the linking group is between 3 and 200 atoms, between 3 and 150 atoms, between 3 and 100 atoms, or between 3 and 50 atoms. Examples of suitable linking groups include alkyl groups, heteroalkyl groups, alkylaryl groups, oligo- and polyethylene glycol chains, and oligo- and poly(amino acid) chains. Variation of the linking group provides additional control over the release of the agents in vivo. In embodiments where the spacer includes a linking group, one or more organic functional groups will generally be used to connect the linking group to both the anti-inflammatory agent and the dendrimers.

Reactions and strategies useful for the covalent conjugation of agents to dendrimers are known in the art. See, for example, March, “Advanced Organic Chemistry,” 5th Edition, 2001, Wiley-Interscience Publication, New York) and Hermanson, “Bioconjugate Techniques,” 1996, Elsevier Academic Press, U.S.A. Appropriate methods for the covalent conjugation of a given agent can be selected in view of the linking moiety desired, as well as the structure of the agents and dendrimers as it relates to compatibility of functional groups, protecting group strategies, and the presence of labile bonds.

The amount of active agent in the dendrimer-active agent conjugates (drug loading) depends on many factors, including the choice of active agent, dendrimer structure and size, and tissues to be treated. In some embodiments, the one or more cannabinoids or cannabinoid derivatives are conjugated to the dendrimer at a concentration between about 0.01% and about 45%, inclusive; between about 0.1% and about 30%, inclusive; between about 0.1% and about 20%, inclusive; between about 0.1% and about 10%, inclusive; between about 1% and about 10%, inclusive; between about 1% and about 5%, inclusive; between about 3% and about 20% by weight, inclusive; or between about 3% and about 10% by weight, inclusive. However, specific drug loading for any given active agent, dendrimer, and site of target can be identified by routine methods, such as those described.

In some embodiments, the conjugation of agents/spacers occurs via about 1%, 2%, 3%, 4%, or 5% of the total available surface functional groups, such as hydroxyl groups, of the dendrimers prior to the conjugation. In other embodiments, the conjugation of agents/spacers occurs on less than 5%, less than 10%, less than 15%, less than 20%, less than 25%, less than 30%, less than 35%, less than 40%, less than 45%, less than 50%, less than 55%, less than 60%, less than 65%, less than 70%, less than 75% total available surface functional groups of the dendrimers prior to the conjugation with active agents. In some embodiments, dendrimer-active agent conjugates retain an effective amount of surface functional groups for targeting to target neural and/or glial cells, whilst conjugated to an effective amount of agents for treat, prevent, and/or image the disease or disorder. In some embodiments, dendrimer-active agent conjugates retain an effective amount of active agents for targeting to target neural and/or glial cells and binding to target receptors on the surface or in the interior of the target neural and/or glial cells.

More specific methods for preparing exemplary dendrimer-active agent conjugates are described in the Examples below.

### IV. Pharmaceutical Formulations

Pharmaceutical compositions including dendrimer-active agent conjugates may be formulated in a conventional manner using one or more physiologically acceptable carriers, optionally including excipients and auxiliaries which facilitate processing of the active compounds into preparations which can be used pharmaceutically, for oral, mucosal (intranasal, buccal, sublingual, vaginal, rectal or pulmonary), transdermal, or injection (intravenous, subcutaneous, intraperitoneal, intramuscular, or intrathecal administration).

The composition, method of, and relative amounts for the formulation is dependent upon the route of administration chosen. The compositions can be stored lyophilized in single use vials for rehydration immediately before use. Other means for rehydration and administration are known to those skilled in the art.

Representative excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are preferably selected from materials which are generally recognized as safe (GRAS) and may be administered to an individual without causing undesirable biological side effects or unwanted interactions.

Generally, pharmaceutically acceptable salts can be prepared by reaction of the free acid or base forms of an agent with a stoichiometric amount of the appropriate base or acid in water or in an organic solvent, or in a mixture of the two; generally, non-aqueous media like ether, ethyl acetate, ethanol, isopropanol, or acetonitrile are preferred. Pharmaceutically acceptable salts include salts of an agent derived from inorganic acids, organic acids, alkali metal salts, and alkaline earth metal salts as well as salts formed by reaction of the drug with a suitable organic ligand (e.g., quatemary ammonium salts). Lists of suitable salts are found, for example, in Remington's Pharmaceutical Sciences, 20th ed., Lippincott Williams & Wilkins, Baltimore, MD, 2000, p. 704. Examples of ophthalmic drugs sometimes administered in the form of a pharmaceutically acceptable salt include timolol maleate, brimonidine tartrate, and sodium diclofenac.

The compositions are preferably formulated in dosage unit form for ease of administration and uniformity of dosage. The phrase “dosage unit form” refers to a physically discrete unit of conjugate appropriate for the patient to be treated. It will be understood, however, that the total single administration of the compositions will be decided by the attending physician within the scope of sound medical judgment. The therapeutically effective dose can be estimated initially either in cell culture assays or in animal models, usually mice, rabbits, dogs, or pigs. The animal model is also used to achieve a desirable concentration range and route of administration. Such information should then be useful to determine effective doses and routes for administration in humans. Therapeutic efficacy and toxicity of conjugates can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index, and is expressed as the ratio, LD₅₀/ED₅₀. Pharmaceutical compositions which exhibit large therapeutic indices are preferred. The data obtained from cell culture assays and animal studies can be used in formulating a range of dosages for human use.

In certain embodiments, the compositions are administered locally, for example, by injection directly into a site to be treated. In some embodiments, the compositions are injected, topically applied, or otherwise administered directly into the vasculature onto vascular tissue at or adjacent to a site of injury, surgery, or implantation. For example, in embodiments, the compositions are topically applied to vascular tissue that is exposed, during a surgical procedure. Typically, local administration causes an increased localized concentration of the compositions, which is greater than that which can be achieved by systemic administration.

Pharmaceutical compositions formulated for administration by parenteral (intramuscular, intraperitoneal, intravenous, or subcutaneous injection) and enteral routes of administration are described.

In some embodiments, dendrimer conjugation may increase the effectiveness and durability of the treatment, which may reduce the need for repeated administration to once per week, once per month, once per six months, once per year, or other longer-term dosing regimens. Some embodiments may be incorporated into drug delivery systems (e.g., implants, pumps, patches, creams, etc) in order to provide controlled, sustained delivery in a manner that reduces the need for compliance and the potential for abuse.

### A. Parenteral Administration

The compositions of dendrimer-active agent conjugates can be administered parenterally. The phrases “parenteral administration” and “administered parenterally” are art-recognized terms and include modes of administration other than enteral and topical administration. The dendrimers can be administered orally, intranasally, subcutaneously, intraperitoneally, or intramuscularly. For liquid formulations, pharmaceutically acceptable carriers may be, for example, aqueous or non-aqueous solutions, suspensions, emulsions, or oils. Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media. The dendrimers can also be administered in an emulsion, for example, water in oil. Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Formulations suitable for parenteral administration can include antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Intravenous vehicles can include fluid and nutrient replenishers, electrolyte replenishers such as those based on Ringer's dextrose. In general, water, saline, aqueous dextrose and related sugar solutions, and glycols such as propylene glycols or polyethylene glycols are preferred liquid carriers, particularly for injectable solutions.

Injectable pharmaceutical carriers for injectable compositions are well-known to those of ordinary skill in the art (see, e.g., Pharmaceutics and Pharmacy Practice, J. B. Lippincott Company, Philadelphia, PA, Banker and Chalmers, eds., pages 238-250 (1982), and ASHP Handbook on Injectable Drugs, Trissel, 15th ed., pages 622-630 (2009)).

### B. Enteral Administration

The compositions of dendrimer-active agent conjugates can be administered enterally (orally, vaginally, rectally, buccally, intranasally, pulmonarily, or transdermally). The carriers or diluents may be solid carriers such as capsule or tablets or diluents for solid formulations, liquid carriers or diluents for liquid formulations, or mixtures thereof.

For liquid formulations, pharmaceutically acceptable carriers may be, for example, aqueous or non-aqueous solutions, suspensions, emulsions, or oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Vehicles include, for example, sodium chloride solution, Ringer's dextrose, dextrose and sodium chloride, lactated Ringer's and fixed oils. Formulations include, for example, aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Vehicles can include, for example, fluid and nutrient replenishers, electrolyte replenishers such as those based on Ringer's dextrose. In general, water, saline, aqueous dextrose and related sugar solutions are preferred liquid carriers. These can also be formulated with proteins, fats, saccharides, and other components of infant formulas.

Oral formulations may be in the form of chewing gum, gel strips, tablets, capsules, or lozenges. Encapsulating substances for the preparation of enteric-coated oral formulations include cellulose acetate phthalate, polyvinyl acetate phthalate, hydroxypropyl methylcellulose phthalate and methacrylic acid ester copolymers. Solid oral formulations such as capsules or tablets are preferred. Elixirs and syrups also are well known oral formulations.

Formulations for administration to mucosal surfaces such as the nose, buccal surfaces or pulmonary, typically contain pharmaceutically acceptable excipients such as those used for parenteral administration, alone or in combination with various surfactants, penetration enhancers, etc.

The compositions can also be made into aerosol formulations (i.e., they can be “nebulized”) to be administered via inhalation. Aerosol formulations can be placed into pressurized acceptable propellants, such as dichlorodifluoromethane, propane, nitrogen, and air. For administration by inhalation, the compounds are delivered in the form of an aerosol spray presentation from pressurized packs or a nebulizer, with the use of a suitable propellant.

### V. Methods of Use

In preferred embodiments, the dendrimer cannabinoid compositions traverse the barrier interfaces of the nervous system, and selectively target specific cells and specific receptors on the cells to alleviate symptoms associated with a variety of diseases, disorders, and conditions. The methods include administering to a subject in need, the compositions in an amount effective to increase permeability of the cannabinoid agents across the barrier interfaces of the nervous system, and/or increase binding of the cannabinoid agents at specific receptors in specific cells, particularly the CB1 receptors and CB2 receptors on cells of the central and peripheral nervous system.

### A. Methods of Treatment

The compositions are suitable to treat disorders associated with neuropathic pain, inflammation, nausea and vomiting, spasticity, and behavioral abnormalities, particularly those that extend to the central and peripheral nervous system. See, for example, Montero-Oleas, et al., “Therapeutic use of cannabis and cannabinoids: an evidence mapping and appraisal of systematic reviews” BMC Complement Med Ther. 20(1):12 (2020) doi: 10.1186/s12906-019-2803-2.

Typically, an effective amount of dendrimer complexes including a combination of a dendrimer with one or more therapeutic, prophylactic, and/or diagnostic active agents are administered to an individual in need thereof. The dendrimers may also include a targeting agent for delivery to target tissue and cells in the spinal cord, the brain, and related areas.

In some embodiments, the dendrimer complexes include an agent that is attached or conjugated to dendrimers, which are capable of preferentially releasing the drug at the target receptor. The agent can be either covalently attached or intra-molecularly dispersed or encapsulated. The amount of dendrimer complexes administered to the subject is selected to deliver an effective amount to reduce, prevent, or otherwise alleviate one or more clinical or molecular symptoms of the disease or disorder to be treated compared to a control, for example, a subject treated with the active agent without dendrimer.

### 1. Site-Specific Targeting

The compositions and methods are designed to circumvent existing challenges in cannabinoid family of receptors-selective, cell-selective drug delivery to the central and peripheral nervous system, and the different organs in the body. The cannabinoid receptors are expressed in various cells and organs in the body, working as a system to regulate many aspects of our functioning. We utilize the small size, and unique bio[physical properties of the dendrimers to bring these drugs to specific cells and receptors in the body. The compositions and methods may increase drug bioavailability in the central and peripheral nervous system by one or more of the following: (i) increasing drug levels at specific cells of interests and specific cannabinoid receptors on or inside them, across brain barriers, particularly the blood-brain and blood-cerebrospinal fluid barriers, (ii) increasing drug solubility, (iii) facilitating target engagement i.e., increasing site-specific binding, (iv) improving drug pharmacokinetics, and (v) reducing off target effects. For example, in some forms, the compositions and methods permit selective delivery of compounds to the peripheral nervous system, by minimizing drug uptake to the brain, thereby increasing the potential of the compositions to be used to selectively treat periphery-specific diseases and disorders, including but not limited to neuropathic pain, regional anesthesia, traumatic nerve injury, and inherited and inflammatory neuropathies.

a. Improve Drug Permeability Across Barrier Interfaces

The dendrimer compositions and methods may improve the delivery of the cannabinoids across one or more of the barrier interfaces in the brain and nervous system, particularly the Blood-Brain Barrier (BBB), the CSF-blood barrier, and the blood-nerve barrier, and to specific receptors on neurons, microglia or astrocytes. These barrier interfaces typically protect neurons from blood-borne substances and help maintain water homeostasis and appropriate milieu for neuronal function in the blood. Due to the clinical significance of cannabinoids, the dendrimer compositions may be used to deliver cannabinoids with improved permeability across these barrier interfaces for site-specific targeting.

i. The Blood-Brain Barrier

The “Blood-Brain Barrier” (BBB) is a continuous endothelial membrane that, along with pericytes and other components of the neurovascular unit, limits the entry of toxins, pathogens, and blood cells to the brain. However, the BBB also represents an obstacle in the delivery of drugs to the central nervous system (CNS), in part because (1) delivering drugs intended for the brain via systemic routes may result in unacceptably high levels of drugs in the periphery; (2) the complex interplay of cells and molecules that contribute to the BBB's structure and function makes it challenging to determine drug permeability at the BBB, drug distribution in the brain, and target engagement in the brain.

Brain microvascular endothelial cells, pericytes, astrocytes, tight junctions, neurons, and basal membrane construct physically tight brain capillaries in the BBB. The brain capillary endothelial cells do not have fenestrations, which limits the diffusion of small molecules and proteins. Inter-endothelial junctions link the endothelial cells to a continuous barrier, severely restricting the penetration of water-soluble substances. Pericytes, astrocytes and basal membrane surround the endothelial cells and finally form the impermeable BBB. Additionally, efflux transporters are located in brain capillary endothelial cells, which are further obstacles against substances entering the brain. The permeability of the BBB is mainly controlled by inter-endothelial junctions that are protein complexes such as adherens junctions, tight junctions, and gap junctions. Adherens junctions primarily regulate the permeability of the endothelial barrier. Tight junctions play a vital role in sustaining the permeability barrier of epithelial and endothelial cells, which control tissue homeostasis. Gap junctions, composed of six connexin molecules, direct electric, and chemical communication between endothelial cells. Finally, instead of having a static structure, the components of the BBB continuously adapt in response to various physiological changes in the brain. The dendrimer compositions and methods of the present application overcome the aforementioned challenges and are suitable for delivering cannabinoids across the blood-brain barrier via one or more of the above-described transport mechanisms.

Molecules cross the BBB by a paracellular pathway (between adjacent cells) or a transcellular pathway (through the cells). For the paracellular pathway, ions and solutes utilize concentration gradients to pass the BBB by passive diffusion. The transcellular pathway includes different mechanisms such as passive diffusion, receptor-mediated transport, and transcytosis.

The physicochemical factors that influence BBB permeability include molecular weight, charge, lipid solubility, surface activity and relative size of the molecule. BBB permeability can also be influenced by physiological factors such as efflux transporters, e.g., β-glycoprotein (β-gp), enzymatic activity, plasma protein binding and cerebral blood flow. Hydrophilic molecules such as proteins and peptides enter the brain through specific and saturable receptor-mediated transport mechanisms such as glucose transporter-1 (GLUT-1), insulin transporter and transferrin transporter. These endogenous transporters are expressed at the luminal and abluminal endothelial cell membranes. Among these transport mechanisms, receptor-mediated transcytosis has been extensively studied to deliver drugs into the brain. The dendrimer compositions and methods of the present application are suitable for delivering cannabinoids across the blood-brain barrier via one or more of the above-described mechanistic routes.

ii. The Blood-Nerve Barrier (BNB)

The blood-nerve barrier (BNB) defines the physiological space within which the axons, Schwann cells, and other associated cells of a peripheral nerve function, thereby ensuring proper function of peripheral nerves, and maintenance of homeostasis of the endoneurial environment. The BNB consists of the endoneurial microvessels within the nerve fascicle and the investing perineurium. Tight junctions between endothelial cells and between pericytes in endoneurial vasculature isolate the endoneurium from the blood, thus preventing uncontrollable leakage of molecules and ions from the circulatory system to the peripheral nerves. In addition, a diffusion barrier exists within the perineurium formed by tight junctions between the neighboring perineurial cells and basement membranes surrounding each perineurial cell layer. The endoneurial capillaries and the perineurial passage are the restrictive barriers which separate the endoneurial extracellular environment of peripheral nerves from both the epineurial perifascicular space and the systemic circulation, thus protecting the endoneurial microenvironment from drastic concentration changes in the vascular and other extracellular spaces.

For drug targets located in peripheral nerves, the BNB can be problematic because of the potential to restrict or prevent drugs from reaching their site of action, thus negatively affecting drug efficacy. In addition, transporter expression profiles in peripheral nerves can be very different from those in the central nervous system. The dendrimer compositions of the present application may be used to improve permeability of cannabinoids across the BNB, thereby improving delivery of cannabinoids to peripheral nerve targets.

iii. The Blood-CSF Barrier

The composition may be used to improve delivery of cannabinoids to target sites via the blood-cerebrospinal fluid barrier (blood-CSF barrier) and the ventricles. The choroid plexus is a vascular tissue found in all cerebral ventricles. The functional unit of the choroid plexus, composed of a capillary enveloped by a layer of differentiated ependymal epithelium. Unlike the capillaries that form the blood-brain barrier, choroid plexus capillaries are fenestrated and have no tight junctions. The endothelium, therefore, does not form a barrier to the movement of small molecules. Instead, the blood-CSF barrier at the choroid plexus is formed by the epithelial cells and the tight junctions that link them. The other part of the blood-CSF barrier is the arachnoid membrane, which envelops the brain. The cells of this membrane also are linked by tight junctions.

The CSF spaces and the cerebral structures adjacent to CSF compartments are pharmacological targets of interest in CNS diseases. For example, the subarachnoid, perivascular, or periventricular spaces are areas of pathogenic lymphocyte, monocyte, and neutrophil accumulation in neuroinflammatory disorders such as multiple sclerosis and related experimental autoimmune encephalitis, or virus-induced neurological disorders including neuroaids and CMV infection. Foci of B-cells detected in different CNS autoimmune diseases and producing potentially deleterious antibodies are thought to be mainly localized in leptomeninges. Therefore, in some forms, the dendrimer compositions may be used to deliver cannabinoids to areas of interest via the blood-CSF spaces connected with deep cervical lymph nodes for ameliorating or treating symptoms associated with neuroinflammatory disorders.

In some forms, the dendrimer compositions may be used to deliver cannabinoids to target sites for ameliorating or treating symptoms and conditions associated with vascular degeneration. For example, cerebral amyloid angiopathy induces degenerative vascular changes, driven by amyloid beta (Aβ) peptide, cystatin c, transthyretin, or gelsolin deposits around penetrating vessels. The deposits are accessible through interconnected CSF/perivascular spaces.

In some forms, the dendrimer compositions may be used to deliver cannabinoids to target sites for ameliorating or treating symptoms and conditions associated with tumor development. For example, periventricular tumors including meningiomas, pharmacoresistant ependymomas, and leptomeningeal metastases from peripheral primary tumors, are all in direct contact with CSF. The blood-tumor barrier is often considered leaky, as a result of the lessened efficacy of tight junctions that allows contrast enhancement in magnetic resonance imaging. However, many cannabinoids are lipophilic and are prevented from crossing the BBB by multidrug resistance (MDR) efflux proteins controlling the transcellular pathway. In a number of periventricular tumors such as ependymomas, MDR proteins remain well expressed at the blood-tumor barrier. Therefore, in some forms, the dendrimer compositions may be used to leverage pharmacological pressure from the CSF to achieve therapeutic concentrations of cannabinoids within the tumoral tissue.

b. Improve Target-Specific Binding

The dendrimer compositions may be used to deliver cannabinoids with increased binding affinity and specificity to one or more receptors for modulation of the endocannabinoid system, including CB1 receptors, CB2 receptors, TRPV receptors, or combinations thereof. As shown in FIG. 1, the dendrimer compositions may be used to deliver cannabinoids that can selectively impact target cells and specific receptors on the target cells, while being cleared from the off-target organs, including but not limited to the neural and glial cells of the brain, spinal cord, heart, lung, kidney, gut, and joints. The improved binding to specific receptors on target cells, may enable the conjugates to achieve fast onset of efficacy at lower doses. The extent of binding to the target receptor could be tailored by drug linking chemistry to the dendrimer. In exemplary embodiments, hydroxyl-terminated PAMAM dendrimers, glucose dendrimers, and other dendrimers may enhance the delivery of cannabinoids to specific receptors e.g., CB1 specific, CB2 specific, and/or both, thereby permitting increased performance of these drugs. In other forms, these dendrimer cannabinoid conjugates could be used in combination with other classes of drugs to achieve synergistic effects.

In some forms, specific ligands e.g., AEA and 2-AG may be conjugated to the dendrimer compositions to cell-type and receptor-type specific modulation of the endocannabinoid system. The dendrimer compositions may also be used to target specific metabolites e.g., intracellular enzymes in neuronal and glial cells for the modulation of the synthesis and degradation of endocannabinoid ligands. For example, the dendrimer compositions may be used to target PLA and DAG for modulating the synthesis of endocannabinoid ligands, and FAAH and MLGL to modulate the degradation of endocannabinoid ligands.

In some forms, the dendrimer compositions may be engineered to deliver cannabinoids by other routes for the modulation cellular activity in the brain and nervous system. For example, the dendrimer compositions may be engineered to deliver cannabinoids via random diffusion, aggregation to the membrane; and lateral diffusion followed by binding into the membrane-buried ligand-binding sites.

i. The Endocannabinoid System in the CNS

The ECS has emerged as one of the key regulatory mechanisms in the brain controlling multiple events such as mood, pain perception, learning and memory among others. It is also thought to provide a neuroprotective role during traumatic brain injury (TBI) and may be part of the brain's natural compensatory repair mechanism during neurodegeneration. Given the clinical significance of the ECS in drug abuse and dependence, the compositions and methods are suitable for targeting components of the ECS to treat cannabis abuse as well as narcotic drug abuse. In the CNS, eCBs act as retrograde messengers mediating feedback inhibition modulating synaptic plasticity. Activation of the CB1 receptor leads to activation of inwardly rectifying K+ channel conductance, decreases in N-type and P/Q-type voltage-operated Ca2+ channel conductance and eCB production. This results in a decrease of neurotransmitter release at excitatory and inhibitory synapses leading to transient effects, as in depolarization-induced suppression of inhibition (DSI) and depolarization-induced suppression of excitation (DSE) or persistent effects as in long-term depression and potentiation (LTP/LTD) during synaptic plasticity. These events make the ECS a key modulator of synaptic plasticity.

Prolonged exposure to CBT receptor agonists results in rapid attenuation of behavioral responsiveness, termed tolerance, and has been attributed to both a decrease in the ability of the receptor to activate effector pathways (i.e., desensitization) and in the reduction in the number of cell surface-expressed receptors (i.e., internalization). At the molecular level, the agonist-bound GPCR becomes a substrate for G protein coupled receptor kinases (GRKs); these kinases phosphorylate serine and/or threonine residues on GPCR cytoplasmic domains, which then become a high affinity target for β-arrestins, Binding of β-arrestins uncouples G-proteins and stimulates receptor internalization and β-arrestin mediated signaling.

Ligand induced receptor phosphorylation by GRKs can result in very specific and distinct phosphorylation profiles or “bar-codes”. These bar-codes are finely tuned and define which signaling cascades are activated, thus opening up a spectrum of possibilities frequently defined as functional selectivity or ligand bias.

Receptor and β-arrestin interaction and signaling cascades are dependent on specific phosphorylation sites controlled by unique GRKs. Mutation of the putative GRK sites from S426/S430 to alanines result in reduced β-arrestin 2 recruitment and receptor internalization, but enhance interaction with β-arrestin 1 and increased β-arrestin 1 mediated signalingp-arrestin mediated signaling from this biased receptor controls the activation of several cascades including ERK1/2, JNK1/2/3, CREB and P38a. It is important to note that these cascades have been previously linked to the activation of CBT receptors. Activation of these cascades by CB1 receptors and β-arrestins in turn regulate gene expression and protein synthesis.

The compositions and methods may be used to elucidate the physiological roles of β-arrestins in the development of pathway-selective or “biased ligands” with greater therapeutic benefit. The compositions and methods may also be used to investigate signaling from biased CBT receptors such as S426A/S430A aid identification of biased ligands as well as provide important tools to elucidate the mechanisms and roles of CB1 receptor signaling.

The subcellular localization and trafficking of CB1 receptors is highly dynamic, with significant effects on receptor signaling. CB1-G protein mediated signaling occurs at the cell surface and at intracellular compartments. At the cell surface, CB1 receptor ligands modulate the interaction between receptors and β-arrestin as a mechanism to influence β-arrestin mediated signaling. This interaction is initiated at the plasma membrane and can continue into intracellular compartments. Interestingly, these location-specific signaling events appear to be widespread among several GPCRs. For example, the LH receptor, β2 adrenergic receptor and the CB2 receptor can signal from intracellular compartments either by (3-arrestins or G proteins via a “super-complex” ultimately resulting in three different spatio-temporal signaling waves. Constitutive activation also plays a role in their trafficking. CB1 receptor location and trafficking are highly dynamic events that are intimately intertwined with their signaling.

ii. Regulating the Endocannabinoids System

The compositions and methods may be used to pharmacologically manipulate eCB levels or their actions by allosteric modulators, thereby providing improved opportunities to regulate the ECS. eCBs are produced on demand with their synthesis typically triggered via increased intracellular Ca2+ at postsynaptic sites in response to sustained synaptic activity. Major eCBs are rapidly deactivated by reuptake mechanisms and degrading enzymes, including fatty acid amide hydrolase (FAAH) and monoacylglycerol lipase. Among eCBs, the derivatives of arachidonic acid such as AEA and 2-AG are dominant and orthosteric. These ligands are agonists for CB1 and CB2 receptors but bind CB1 receptors with higher affinity (AEA Ki=89 nM and 321 nM for CB1 and CB2 receptors respectively; 2-AG Ki=472 nM and 1400 nM for CB1 and CB2 receptors respectively; Pertwee et al., 2010). Allosteric eCBs have been identified, including pregnenolone and lipoxin A4 which can modulate CB1 receptor signaling with possible therapeutic value (Pamplona et al., 2012; Vallee et al., 2014; Pertwee, 2015). The compositions and methods may be used to aid pharmacological characterization of orthosteric and allosteric modulators to clearly elucidate their physiological roles and modes of action. In some forms, the compositions and methods may be used to pharmacologically manipulate eCB levels or their actions by allosteric modulators, thereby providing improved opportunities to regulate the ECS.

iii. Improve Cannabinoid Receptor Binding

The dendrimer compositions may improve binding to one or both cannabinoid receptors to modulate signaling in a cell-specific and tissue-specific manner.

The identification and cloning of the two major cannabinoid (CB1 and CB2) receptors together with the discovery of their endogenous ligands in the late 80s and early 90s, resulted in a major effort aimed at understanding the mechanisms and physiological roles of the endocannabinoid system (ECS). Due to their expression and localization in the central nervous system (CNS), the CB1 receptor together with its endogenous ligands (endocannabinoids (eCB)) and the enzymes involved in their synthesis and degradation, has been implicated in multiple pathophysiological events ranging from memory deficits to neurodegenerative disorders among others. For example, CB1 receptors are particularly abundant in the frontal cortex, hippocampus, basal ganglia, hypothalamus, cerebellum, spinal cord, and peripheral nervous system. Therefore, in some forms, the dendrimer compositions may be used to deliver cannabinoids to ameliorate or treat symptoms of diseases and disorders affecting one or more of these nervous system areas.

CB1 receptors are also present in both inhibitory GABAergic neurons and excitatory glutamatergic neurons. Thus, in some forms, the compositions may be used to ameliorate or treat symptoms of diseases and disorders associated with dysregulated GABAergic and glutamatergic signaling. CB2 receptors are most abundantly found on cells of the immune system, hematopoietic cells, and glia cells. The CB2 receptors are mainly expressed in the periphery under normal healthy conditions; in conditions of disease or injury, this upregulation occurs within the brain and CB2 receptors are therefore expressed in the brain in unhealthy states. Therefore, in some forms, the compositions may be used to ameliorate or treat symptoms of diseases and disorders associated with damaged immune cells, hematopoietic cells, and glia cells e.g., macrophages and activated microglia. The neurophysiology of the CB1 receptors and CB2 receptors are described in more detail as follows.

a. CB1 Receptors

The CB1 receptor is one of the most abundant G protein-coupled receptors (GPCRs) in the CNS and is found in particularly high levels in the neocortex, hippocampus, basal ganglia, cerebellum, and brainstem. CB1 receptors are also found on peripheral nerve terminals and some extra-neural sites such as the testis, eye, vascular endothelium, and spleen. CB1 receptors are highly enriched at presynaptic and axonal compartments, restricting their function to sites of synaptic activity. In addition to its location on the cell surface, intracellular localization of CB1 receptors has also been reported in heterologous systems and primary cultures. The CB1 receptor binds the main active ingredient of Cannabis sativa (marijuana), Δ9-tetrahydrocannabinol (Δ9-THC) and mediates most of the CNS effects of Δ9-THC. In addition, CB1 receptors bind synthetic cannabimimetic compounds such as CP55940, JWH-015, WIN55212-2 and the endogenous arachidonic acid derivatives arachidonylethanolamine (AEA) and 2-arachidonylglycerol (2-AG). Upon ligand binding and receptor activation, CB1 receptors are primarily coupled to pertussis toxin (PTX)-sensitive Gi/o type G proteins which leads to a rapid decrease in levels of cAMP by inhibiting adenylate cyclase activity. Coupling to other G proteins including Gs, albeit with low efficacy, can also stimulate adenylate cyclase though the extent of accumulation of cAMP is not necessarily a good indicator of G protein coupling. Evidence of promiscuous coupling to different G proteins, signaling roles mediated by β-arrestins and signaling from intracellular compartments adds yet another level of complexity making these receptors, like other GPCRs, pluridimensional. CB1 receptors exhibit constitutive activity indicative of G protein activation in the absence of agonists, and this could mediate their highly polarized localization to axonal and presynaptic compartments.

FIGS. 1A and 1B are schematics of the key enzymes in the cannabinoid pathway. Diacylglycerol lipase (DGLα) and phospholipase D (PLD) produce the endogenous ligands arachidonylethanolamine (AEA) and 2-arachidonylglycerol (2-AG; FIG. 1A). These activate the cannabinoid 1 receptor (CB1) receptor in the central nervous system (CNS). The result can include modulation of adenylate cyclase activity to inhibit cAMP accumulation, voltage-gated calcium channels (VGCC), K+ channels and neurotransmitter release in presynaptic excitatory and inhibitory synapses (FIG. 1A). Following activation of the CB1 receptor by ligand binding, signaling via G protein and/or β-arrestin may occur at the plasma membrane, in endocytic pits or in endosomes after internalization of the receptor (FIG. 1B). G proteins usually bind the unphosphorylated receptor while β-arrestin binds the receptor phosphorylated by G protein receptor kinases (FIG. 1B). FIG. 1C is a schematic of the impact of differential cannabinoid (cb) receptor signaling modalities on neuromodulation of health and disease in specific ways.

b. CB2 Receptors

The CB2 receptor exhibits a more defined pattern of expression in the brain than CB1 receptors, and is found predominantly in cells and tissues of the immune system (Klein, 2005; Mackie, 2006). In the CNS, CB2 receptor expression is associated with inflammation, and it is primarily localized to microglia, resident macrophages of the CNS (Mackie, 2008; Palazuelos et al., 2009). This selective localization together with the modulatory effect of the CB2 receptor on microglia function is particularly relevant since microglial cells have a significant role in Alzheimer's disease (AD) and other diseases associated with the basal ganglia (Ramirez et al., 2005; Sagredo et al., 2007; Femindez-Ruiz et al., 2011; Yeh et al., 2016). Additionally, CB2 receptors expressed in neurons can control synaptic function and are involved in drug abuse and synaptic plasticity (Xi et al., 2011; Stempel et al., 2016). For example, the selective CB2 receptor agonist JWH133 inhibits dopaminergic firing from the ventral tegmental area and reduced cocaine self-administration (Zhang et al., 2016). Furthermore, neuronal CB2 receptors work independently from CB1 receptors to modulate inhibitory plasticity in the CA2/3 regions of the hippocampus and gamma oscillations in vivo (Stempel et al., 2016).

iv. Improve Non-Cannabinoid Receptor Binding

The dendrimer compositions may improve binding of cannabinoids to one or more non-cannabinoid receptors to modulate signaling in a cell-specific and tissue-specific manner. For example, cannabinoids also bind with other G protein-coupled receptors e.g. GPR55, GPR18, GPR3, GPR6, GPR12 receptors; transient receptor potential channels e.g., TRP vanilloids TRPV1 to TRPV4, TRP ankyrin TRPA1, AND TRPM member TRPM8 receptors, peroxisome proliferator-activated receptors e.g. PPAR2 and PPARy; monoamine transporters e.g. norepinephrine, dopamine, and serotonin 1A receptors, fatty acid amide hydrolase, monoacylglycerol lipase, transport fatty acid binding proteins, adenosine equilibrative nucleoside transporters, and glycine receptors al and a3 receptors. Therefore, in some forms, the dendrimer compositions may be used to improve binding of cannabinoids to one or more non-cannabinoid receptors to modulate signaling at one or more of the above-named receptors.

### B. Conditions to be Treated

The compositions are suitable for treating one or more diseases, conditions, and injuries in the central and peripheral nervous system. The compositions can also be used for treatment of a variety of diseases, disorders and injury including gastrointestinal disorders, ocular diseases, and treatment of other tissues where the nerves play a role in the disease or disorder. Cannabinoids have been tested for the treatment of indications including nausea and vomiting due to cheimotherapy, appetite stimulation in HIV/AIDS, chronic pain, spasticity due to multiple sclerosis or paraplegia, depression, anxiety disorder, sleep disorder, psychosis, glaucoma, or Tourette syndrome

The compositions and methods are also suitable for prophylactic use. For example, the compositions may be administered to a patient in need thereof to ameliorate, treat or prevent symptoms related with an inflammatory disease, neurodegenerative disease, pain disorder, spasms and convulsive disorders, bone disorders, psychotic, metabolic disorders as well as cancer-related neuropathic pain. The compositions are particularly useful for treating or ameliorating complications associated with chemotherapy e.g., nausea and vomiting; symptoms of HIV/AIDs e.g., loss of appetite, symptoms associated with Multiple Sclerosis e.g., spasticity; as well as one or more of sleep disruption and/or sleep disorders, anxiety disorders, psychoses, chronic pain, glaucoma, and/or Tourette's syndrome.

The dendrimer conjugate composition, preferably with a diameter under 20 nm and a hydroxyl group surface density at least 0.8 OH groups/nm², preferably under 10 nm and a hydroxyl group surface density of at least 1 OH groups/nm², more preferably under 6 nm and a hydroxyl group surface density of at least 1 OH groups/nm², and most preferably between 5 nm and a hydroxyl group surface density at least 1.5 OH groups/nm², delivering a therapeutic, prophylactic or diagnostic agent, selectively targets microglia and astrocytes, which play a key role in the pathogenesis of many disorders and conditions including neurodevelopmental, neurodegenerative diseases, neuropsychiatric disorders, and chronic pain. Thus, the dendrimer complexes are administered in a dosage unit amount effective to treat or alleviate conditions associated with the pathological conditions of the central and peripheral nervous system. For example, the dendrimer complexes are administered in a dosage unit amount effective to treat or alleviate conditions associated with pathological conditions that affects neurons microglia and astrocytes. Generally, by targeting these cells, the dendrimers deliver agent specifically to treat neuroinflammation.

a. Relief from Chronic Pain

The compositions and methods are suitable for the treatment of chronic pain and diseases and disorders associated with chronic pain.

Chronic pain is a complex sensory, cognitive, and emotional experience that imposes a great personal, psychological, and socioeconomic burden on patients. An estimated 1.5 billion people worldwide are afflicted with chronic pain, which is often difficult to treat and may be resistant to the potent pain-relieving effects of opioid analgesics. Chronic pain conditions are often resistant to standard treatments, including treatment with opioid medications such as morphine.

Cannabinoids exert their actions primarily through Gi/o-protein coupled cannabinoid CB1 and CB2 receptors expressed throughout the nervous system. CBT receptors are found at key nodes along the pain pathway and their activity gates both the sensory and affective components of pain. CB2 receptors are typically expressed at low levels on microglia, astrocytes, and peripheral immune cells. In chronic pain states, there is a marked increase in CB2 expression which modulates the activity of these central and peripheral immune cells with important consequences for the surrounding pain circuitry.

The compositions and methods may be used to target CB1 or CB2 receptors to manage pain and improve pain outcomes in a variety of pain types including but not limited to musculoskeletal pain, osteoarthritis pain, and/or joint pain, as well as other types of pain attributable to inflammation. In some forms, the compositions and methods may be used for management of neuropathic pain and/or nociceptive pain including pain due to cancer, injury, accident, surgery, inflammation, tissue damage, arthritis (including osteoarthritis and rheumatoid arthritis), joint pain, pain from infection, gastrointestinal pain, diabetes, diabetes neuropathy, post-shingles neuralgia, neuropathic pain, peripheral neuropathy or multiple sclerosis. In an exemplary embodiment, the compositions and methods modulate microglia function to facilitate cannabinoid-mediated analgesia via microglia-neuron interactions within the spinal nociceptive circuitry. In another exemplary embodiment, the compositions and methods may be administered in a complementary regimen with opioids, offering additional pain reduction in addition to that provided by the opioids. In these forms, combination therapies for chronic pain may have the benefit of reducing patient pain levels, reducing reliance on (and addiction to) opioids, or both.

b. Mental Health Disorders and Conditions

The compositions and methods are suitable for the treatment of a variety of mental health disorders and conditions including but not limited to affective or mood disorders, anxiety disorders, childhood disorders, eating disorders, personality disorders, schizophrenia and other psychotic disorders, and substance-related disorders.

i. Affective or Mood Disorders

Affective or mood disorders are described by marked disruptions in emotions (severe lows called depression or highs called hypomania or mania). These include bipolar disorder, cyclothymia, hypomania, major depressive disorder, disruptive mood dysregulation disorder, persistent depressive disorder, premenstrual dysphoric disorder, seasonal affective disorder, depression related to medical illness, depression induced by substance use or medication.

ii. Anxiety Disorders

Anxiety disorders differ from normal feelings of nervousness or anxiousness and involve excessive fear or anxiety. Anxiety disorders include generalized anxiety disorder, panic disorder, social anxiety disorder, and various phobia-related disorders.

Generalized anxiety disorder (GAD) usually involves a persistent feeling of anxiety or dread, which can interfere with daily life. It is not the same as occasionally worrying about things or experiencing anxiety due to stressful life events. People living with GAD experience frequent anxiety for months, if not years. The compositions and methods are suitable for the treatment of one or more symptoms of GAD, including but not limited to restlessness, fatigue, difficulty concentrating, irritability, headaches, muscle aches, stomach aches, or unexplained pains, excessive worry, sleep issues e.g., difficulty falling or staying asleep.

Panic Disorder is an anxiety disorder characterized by unexpected and repeated episodes of intense fear accompanied by physical symptoms that may include chest pain, heart palpitations, shortness of breath, dizziness, or abdominal distress, or sense of losing control even when there is no clear danger or trigger. Individuals with panic disorders often worry about when the next attack will happen and actively try to prevent future attacks by avoiding places, situations, or behaviors they associate with panic attacks. Panic attacks can occur as frequently as several times a day or as rarely as a few times a year. The compositions and methods are suitable for the treatment of one or more symptoms of panic attacks including but not limited to heart palpitations, excess sweating, trembling, or tingling, chest pain, and difficulty controlling feelings e.g., feelings of impending doom and feelings of being out of control.

Social anxiety disorder is an intense, persistent fear of being watched and judged by others. For people with social anxiety disorder, the fear of social situations may feel so intense that it seems beyond their control. For some people, this fear may get in the way of going to work, attending school, or doing everyday things. The compositions and methods are suitable for the treatment of one or more symptoms of social anxiety disorders including but not limited to excess blushing, sweating, or trembling, heart palpitations, stomachaches, rigid body posture or speaking with an overly soft voice, and feelings of self-consciousness or fear of negative judgement.

A phobia is an intense fear of or aversion to—specific objects or situations. Although it can be realistic to be anxious in some circumstances, the fear people with phobias feel are out of proportion to the actual danger caused by the situation or object. The compositions and methods are suitable for the treatment of one or more symptoms of phobias including but not limited to irrational or excessive worry about encountering the feared object or situation, immediate intense anxiety upon encountering the feared object or situation, and enduring unavoidable objects and situations with intense anxiety.

iii. Eating Disorders

Eating disorders are serious and often fatal illnesses that are associated with severe disturbances in people's eating behaviors and related thoughts and emotions. Eating disorders include preoccupation with food, body weight, and shape. Common eating disorders include anorexia nervosa, bulimia nervosa, and binge-eating disorder.

Anorexia nervosa is a condition where people avoid food, severely restrict food, or eat very small quantities of only certain foods. They also may weigh themselves repeatedly. Even when dangerously underweight, they may see themselves as overweight. There are two subtypes of anorexia nervosa: a restrictive subtype and a binge-purge subtype. People with the restrictive subtype of anorexia nervosa severely limit the amount and type of food they consume. People with the binge-purge subtype of anorexia nervosa also greatly restrict the amount and type of food they consume. In addition, they may have binge-eating and purging episodes-eating large amounts of food in a short time followed by vomiting or using laxatives or diuretics to get rid of what was consumed.

Symptoms of anorexia nervosa including but not limited to thinning of the bones (osteopenia or osteoporosis), mild anemia and muscle wasting and weakness, brittle hair and nails, dry and yellowish skin, growth of fine hair all over the body (lanugo), severe constipation, low blood pressure, slowed breathing and pulse, damage to the structure and function of the heart, brain damage, multiorgan failure, drop in internal body temperature, causing a person to feel cold all the time, lethargy, sluggishness, or feeling tired all the time, and infertility.

Bulimia nervosa is a condition where people have recurrent and frequent episodes of eating unusually large amounts of food and feeling a lack of control over these episodes. This binge-eating is followed by behavior that compensates for the overeating such as forced vomiting, excessive use of laxatives or diuretics, fasting, excessive exercise, or a combination of these behaviors. People with bulimia nervosa may be slightly underweight, normal weight, or over overweight. Symptoms of bulimia nervosa include chronically inflamed and sore throat, swollen salivary glands in the neck and jaw area, worn tooth enamel and increasingly sensitive and decaying teeth as a result of exposure to stomach acid, acid reflux disorder and other gastrointestinal problems, intestinal distress and irritation from laxative abuse, severe dehydration from purging of fluids, electrolyte imbalance (too low or too high levels of sodium, calcium, potassium, and other minerals) which can lead to stroke or heart attack.

The compositions are particularly useful for administration to patients in need thereof to stimulate appetite, reduce nausea, and vomiting. For example, lack of appetite is a common symptom present in patients with opportunistic infections e.g., HIV/AIDS, and patients undergoing chemotherapy and/or other treatments for cancer. Opportunistic infections are infections to which people with a weakened immune system are susceptible. Furthermore, some medications may decrease appetite because of side effects, such as tingling around the mouth, nausea, and changes in the sense of taste. In addition, depression resulting from terminal diseases and/or treatments of terminal diseases can cause a loss of appetite. Therefore, in some forms, the compositions can be administered to such patients to stimulate appetite and/or reduce nausea or wasting syndrome.

c. Neurological and Neurodegenerative Diseases

The compositions and methods are suitable for the treatment of symptoms associated with neurological and neurodegenerative diseases.

Neurodegenerative diseases are chronic progressive disorders of the nervous system that affect neurological and behavioral function and involve biochemical changes leading to distinct histopathologic and clinical syndromes (Hardy H, et al., Science. 1998; 282:1075-9). Abnormal proteins resistant to cellular degradation mechanisms accumulate within the cells. The pattern of neuronal loss is selective in the sense that one group gets affected, whereas others remain intact. Often, there is no clear inciting event for the disease. The diseases classically described as neurodegenerative are Alzheimer's disease, Huntington's disease, and Parkinson's disease.

Neuroinflammation, mediated by activated microglia and astrocytes, is a major hallmark of various neurological disorders making it a potential therapeutic target (Hagberg, H et al., Annals of Neurology 2012, 71, 444; Vargas, D L et al., Annals of Neurology 2005, 57, 67; and Pardo, C A et al., International Review of Psychiatry 2005, 17, 485). Multiple scientific reports suggest that mitigating neuroinflammation in early phase by targeting these cells can delay the onset of disease and can in turn provide a longer therapeutic window for the treatment (Dommergues, M A et al., Neuroscience 2003, 121, 619; Perry, V H et al., Nat Rev Neurol 2010, 6, 193; Kannan, S et al., Sci. Transl. Med. 2012, 4, 130ra46; and Block, M L et al., Nat Rev Neurosci 2007, 8, 57). The delivery of therapeutics across blood brain barrier is a challenging task. The neuroinflammation causes disruption of blood brain barrier (BBB). The impaired BBB in neuroinflammatory disorders can be utilized to transport drug loaded nanoparticles across the brain (Stolp, H B et al., Cardiovascular Psychiatry and Neurology 2011, 2011, 10; and Ahishali, B et al., International Journal of Neuroscience 2005, 115, 151).

The compositions and methods can also be used to deliver active agents for the treatment of a neurological or neurodegenerative disease or disorder or central nervous system disorder. In preferred embodiments, the compositions and methods are effective in treating, and/or alleviating neuroinflammation associated with a neurological or neurodegenerative disease or disorder or central nervous system disorder. The methods typically include administering to the subject an effective amount of the composition to increase cognition or reduce a decline in cognition, increase a cognitive function or reduce a decline in a cognitive function, increase memory or reduce a decline in memory, increase the ability or capacity to learn or reduce a decline in the ability or capacity to learn, or a combination thereof.

Neurodegeneration refers to the progressive loss of structure or function of neurons, including death of neurons. For example, the compositions and methods can be used to treat subjects with a disease or disorder, such as Parkinson's Disease (PD) and PD-related disorders, Huntington's Disease (HD), Amyotrophic Lateral Sclerosis (ALS), Alzheimer's Disease (AD) and other dementias, Prion Diseases such as Creutzfeldt-Jakob Disease, Corticobasal Degeneration, Frontotemporal Dementia, HIV-Related Cognitive Impairment, Mild Cognitive Impairment, Motor Neuron Diseases (MND), Spinocerebellar Ataxia (SCA), Spinal Muscular Atrophy (SMA), Friedreich's Ataxia, Lewy Body Disease, Alpers' Disease, Batten Disease, Cerebro-Oculo-Facio-Skeletal Syndrome, Corticobasal Degeneration, Gerstmann-Straussler-Scheinker Disease, Kuru, Leigh's Disease, Monomelic Amyotrophy, Multiple System Atrophy, Multiple System Atrophy With Orthostatic Hypotension (Shy-Drager Syndrome), Multiple Sclerosis (MS), Neurodegeneration with Brain Iron Accumulation, Opsoclonus Myoclonus, Posterior Cortical Atrophy, Primary Progressive Aphasia, Progressive Supranuclear Palsy, Vascular Dementia, Progressive Multifocal Leukoencephalopathy, Dementia with Lewy Bodies (DLB), Lacunar syndromes, Hydrocephalus, Wemicke-Korsakoff's syndrome, post-encephalitic dementia, cancer and chemotherapy-associated cognitive impairment and dementia, and depression-induced dementia and pseudodementia.

In some embodiments, the subject has an excitotoxicity disorder. Excitotoxicity is a process through which nerve cells become damaged because they are overstimulated. A number of conditions are linked with excitotoxicity including stroke, traumatic brain injury, multiple sclerosis, amyotrophic lateral sclerosis, Alzheimer's disease, and spinal injuries. Damage to the nerve cells results in corresponding neurological symptoms which can vary depending on which cells are damaged and how extensive the damage is. Once damaged, nerve cells cannot be repaired, and the patient can experience permanent impairment. A number of drugs have been developed and used in an attempt to interrupt, influence, or temporarily halt the glutamate excitotoxic cascade toward neuronal injury. One strategy is the “upstream” attempt to decrease glutamate release. Thus, in some embodiments, the dendrimer complexes include one or more active agent for treating excitotoxicity disorder.

In some embodiments, the subject has a nervous system disorder or is in need of neuroprotection. Exemplary conditions and/or subjects include, but are not limited to, subjects having had, subjects with, or subjects likely to develop or suffer from a stroke, a traumatic brain injury, a spinal cord injury, Post-Traumatic Stress syndrome, or a combination thereof.

In some embodiments, the compositions and methods are administered to a subject in need thereof in an effective amount to reduce, or prevent one or more molecular or clinical symptoms of a neurodegenerative disease, or one or more mechanisms that cause neurodegeneration. Exemplary neurological and neurodegenerative disorders are further described below.

i. Multiple Sclerosis (MS)

The compositions and methods may be useful for treating, and/or alleviating symptoms associated with MS. MS is a major immune-related neurodegenerative disease characterized by demyelinization with axonal and neuronal loss. Cannabis, Δ9-THC or other CB agonist improves spasticity, spasms and pain among other signs of MS (Croxford, 2003; Pertwee, 2007; Rog, 2010; Notcutt et al., 2012). Use of Sativex® (Nabiximol) an oromucosal spray of cannabis extract containing fixed concentrations of Δ9-THC and cannabidiol (CBD), results in symptomatic improvement in patients with MS. There is a reduction in motor dysfunction and pain, observed in meta-analysis of several clinical studies. Therefore, in some forms, the compositions may be administered to a patient in need thereof, to reduce muscle spasticity and pain associated with multiple sclerosis. In some forms, the compositions may be administered to a patient in need thereof to reduce inflammation related fatigue, which may, in turn, improve mobility in MS patients.

At the molecular level, these improvements are generally linked to the activation of both CB1 receptors and CB2 receptors by agonists, resulting in their dual anti-inflammatory and neuroprotective effects throughout the CNS. These effects include up-regulation of prosurvival molecules such as interleukins in astroglia, and the reduction of cytotoxic factors such as nitric oxide, reactive oxygen species and proinflammatory cytokines in microglia. The precise mechanisms by which receptors exert their neuroprotective activity might include activation of phosphatidylinositol 3-kinase/mammalian target of rapamycin complex 1 (mTOR1) pathway and brain-derived neurotrophic factor (BDNF).

Synthetic CBs reduce inflammation and neuropathic pain in the Experimental Autoimmune Encephalomyelitis (EAE) mouse model. Similar results were observed with systemic treatment with the agonists, WIN55212-2, ACEA and JWH-015 of mice with established Theiler's Murine Encephalomyelitis Virus-induced Demyelinating Disease, a mouse model of chronic progressive MS. Mouse motor function was improved by modulating microglia and lymphocyte infiltration into the spinal cord. In contrast, when an inverse agonist of the CB1 receptor (SR141716A) was applied, the EAE was worsened likely by releasing pro-inflammatory cytokines in the mouse brain and spinal cord. Taken together, these results suggest that in MS, the neuroprotective roles of CBT and CB2 receptors might be impaired, and their enhancement could provide new therapeutic approaches.

Therefore, the compositions and methods may be effective for treating and/or alleviating one or more symptoms of MS including neuroinflammation, pain, muscle spasms, fatigue, stiffness and weakness, depression and anxiety, and speech and swallowing difficulties.

ii. Huntington's Disease (HD)

Dysregulation of the ECS is also reported in experimental models and patients with HD. The CBT receptor expression is reduced, at least somewhat (e.g., 27% decrease in the striatum of the CB1 receptor mRNA), prior to symptoms of neurodegenerative HD in mice (McCaw et al., 2004). Losing the CB1 receptor expression decreases motor performance and increases the amount of aggregates in the striatum of HD mice (Mievis et al., 2011). Major loss of CB1 receptors is also reported in patients with HD (Glass et al., 2000). Activation of the CB1 receptor may help reduce the progression of HD. For example, preclinical evidence suggested the use of CBs such as Sativex® for neuroprotection in patients with progressive neurodegenerative conditions like HD (Valdeolivas et al., 2012). Furthermore, selected receptor agonists have neuroprotective potential in a cell culture model of HD (Scotter et al., 2010; Laprairie et al., 2016). Ligands biased to $-arrestin mediated signaling such as Δ9-THC, reduced cellular function and viability in these models, suggesting a potential pharmacological profile for therapeutic agonists (Laprairie et al., 2014, 2016). These events are mediated in part by the activation of Gai/o mediated pathways and might limit glutamate release from cortical neurons and GABA from striatal medium spiny neurons (Dowie et al., 2010; Laprairie et al., 2016). Results obtained investigating the R6/2 mouse model of HD, indicate that CB1 receptor activation parallels BDNF expression leading to neuroprotection (Blizquez et al., 2015). In general, the in vivo and in vitro data suggest that CB agonist with specific pharmacological profiles (biased towards BDNF upregulation and release) could be developed to treat or ameliorate HD.

Therefore, the compositions and methods may be effective for reducing the progression of HD in a subject by increasing neuroprotection in the subject via one or more of the above-described mechanisms. The compositions and methods may also be effective for treating and/or alleviating one or more symptoms of HD including but not limited to neuroinflammation, pain, involuntary jerking, or writhing movements (chorea), muscle problems, such as rigidity or muscle contracture (dystonia), slow or unusual eye movements, impaired gait, posture and balance, difficulty with speech or swallowing, cognitive impairments, and behavioral abnormalities.

iii. Alzheimer's Disease

CBT receptors have also been the focus of intense research as a potential target in AD. This work has been performed in vitro, animal models and post-mortem samples. Changes in the expression levels of several components of the ECS in post-mortem samples from AD patients have been identified, although their role in the pathophysiology of the disorder is still unknown. For example, CBT receptors in hippocampus from patients with AD were not different from aged-matched controls. However, the levels of MAGLs, the degradative enzyme of 2-AG, were reduced at their site of action in these patients, suggesting an altered eCB signaling and architecture (Mulder et al., 2011). Limited positive behavioral results have been observed in small clinical trials and pilot studies using analogs of Δ9-THC (Aso and Ferrer, 2014). Analysis of the studies and trials available, suggest significant benefits from synthetic CBs on some of the behavioral and psychological symptoms of dementia (Liu et al., 2015). However, these conclusions were based on short and limited studies; further work will be needed to assess the safety and efficacy of CBs in AD. In experimental models of AD, several findings indicate that the activation of both CB1 receptors and CB2 receptors might have beneficial effects mainly through neuroprotection against Aβ toxicity as previously noted for other neurodegenerative disorders. (Stumm et al., 2013). Tau protein phosphorylation is also reduced by CBD in PC12 cells, providing a different neuroprotective mechanism during AD (Esposito et al., 2006). Since CB1 receptors are not likely directly activated by CBD, the impact on Tau phosphorylation may be via the antioxidant effect of CBD or perhaps as a CB receptor independent effect. A reduction in harmful 0-amyloid peptide and tau phosphorylation, while promoting intrinsic CNS repair mechanisms may take place consecutively due to activation of the immune and CNS CB system in AD (Aso and Ferrer, 2014). For example, recent work on the TREM2 receptor in microglia, where CB2 receptors are expressed and control cellular responses, also provides an immune related mechanism to control AD (Yeh et al., 2016).

Aging is a major risk factor for neurodegenerative diseases and neuronal progenitor cell proliferation is greatly reduced in the process. Remarkably, CBs can stimulate embryonic and adult neurogenesis (Jiang et al., 2005; Trazzi et al., 2010). Axonal guidance, cell migration, synapse formation and cell survival are also modulated during development. Dysregulation of these processes during development and aging could significantly contribute to multiple disorders of the CNS.

iv. Traumatic Brain Injury

The CB1/2 receptors are involved in TBI and that 2-AG increases after TBI in animal models. There is an “on-demand” signal to generate eCB following TBI that can decrease brain edema and inflammation. These events may be neuroprotective and prevent excitotoxicity, inhibit inflammatory cytokine production and augment stem cell migration and differentiation. Furthermore, CB1 receptor and CB2 receptor antagonists prevent drug-induced neuroprotection in a mouse mode of TB1. However, as indicated previously for other disorders, limited clinical data is available to support efficacy and safety of CBs during TBI. Therefore, the compositions and methods may also be effective for treating and/or alleviating one or more symptoms of TBT including but not limited to neuroinflammation, pain, cognitive impairments, behavioral abnormalities, fatigue, problems with speech, nausea or vomiting, headache, sensory impairments, and sleep abnormalities.

d. Gastrointestinal Disorders

The endocannabinoid system (i.e., endogenous circulating cannabinoids) performs protective activities in the GI tract. Therefore, the compositions and methods may be effective for treating and/or alleviating various GI conditions such as necrotizing enterocolitis (NEC), abdominal sepsis, pneumonia, arthritis, pancreatitis and atherosclerosis, inflammatory bowel disease (especially Crohn's disease), irritable bowel syndrome, and secretion and motility-related disorders, the compositions and methods may also be effective for treating and/or alleviating one or more symptoms of gastrointestinal dysfunction in the gut, liver, and pancreas, including but not limited to nausea and vomiting, cannabinoid hyperemesis syndrome, anorexia, weight loss, and chronic abdominal pain.

In some embodiments, a singular dendrimer complex composition can simultaneously treat, and/or diagnose multiple symptoms at two distinct locations of a human body including the gastrointestinal track and the central nervous system. For example, the dendrimer complex composition, including a dendrimer linked to a therapeutic, prophylactic or diagnostic agent, can treat the gastrointestinal area via enteral administration whilst selectively targeting to microglia and astrocytes after absorption into the blood stream. Microglia and astrocytes play a key role in the pathogenesis of NEC.

e. Glaucoma and Vision Disorders

Glaucoma is one of the leading causes of blindness in the world. Despite the diverse therapeutic possibilities, new and better treatments for glaucoma are highly desirable. Elevated intraocular pressure results from blockage in the flow of fluid that helps the eye maintain its rigid shape and represents a primary risk factor for glaucoma development and progression. Normally this clear fluid, called the aqueous humor, circulates between the front of the lens and the back of the comea. In people with elevated intraocular pressure the outflow of fluid from the anterior chamber of the eye becomes restricted, causing pressure to build up like water behind a dam. Increased pressure in the eye contributes to glaucoma by decreasing the flow of nutrients to the optic nerve.

Therefore, the compositions may be administered to a patient in need thereof to lower the intraocular pressure (IOP) and provide neuroprotection. For example, a composition containing a THC and/or THC analog may be administered reduce IOP while a composition containing HU-211 may be administered to provide protection to nerves. In some forms, the compositions can be administered to a patient in need thereof to protect or rescue the optic nerve from damage and/or restore its blood supply.

f. Sleep Disruption and Sleep Disorders

CB1 receptors located in the pons and basal forebrain may be involved in sleep induction. This process is possibly related to activation of cholinergic neurons located in the basal forebrain and pons via CB1 receptors, assisting in the induction of sleep. CB1 receptors enhances the activation of the serotonergic system yielding a potential regulatory role in the sleep-wake cycle.

Therefore, the compositions may be administered to a patient in need thereof to improve sleep architecture. In some forms, the compositions may increase total sleep time, decrease wake after sleep onset, decrease slow wave sleep, and/or reduce, or increase latency to REM sleep as needed, and/or increase sleepiness in patients suffering from insomnia. In some forms, the compositions may be administered to increase alertness in a patient e.g., a patient suffering from narcolepsy.

### C. Dosage and Effective Amounts

In some in vivo approaches, the dendrimer complexes are administered to a subject in a therapeutically effective amount. The term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease or disorder, and the treatment being affected.

Generally, the dose of the compositions can be about 0.001 to about 100 mg/kg body weight of the subject being treated, from about 0.01 to about 100 mg/kg body weight, from about 0.1 mg/kg to about 10 mg/kg, and from about 0.5 mg to about 5 mg/kg body weight. The subjects are typically mammals, most preferably, humans.

For example, dendrimer complex compositions can be in an amount effective to deliver one or more active agents to cells at or nearby the site of inflammation, particularly inflammation of the central nervous system, or inflammation of the eye. Therefore, in some embodiments, the dendrimer complex compositions including one or more active agent are in an amount effective to ameliorate inflammation in a subject. In a preferred embodiment, the effective amount of dendrimer complex compositions does not induce significant cytotoxicity in the cells of a subject compared to an untreated control subject. Preferably, the amount of dendrimer complex compositions is effective to prevent or reduce inflammation and/or further associated symptoms of a disease or disorder in a subject compared to an untreated control.

In general, the timing and frequency of administration will be adjusted to balance the efficacy of a given treatment or diagnostic schedule with the side-effects of the given delivery system. Exemplary dosing frequencies include continuous infusion, single and multiple administrations such as hourly, daily, or weekly dosing.

In some embodiments, dosages are administered once, twice, or three times daily, or every other day, two days, three days, four days, five days, or six days to a human. In some embodiments, dosages are administered about once or twice every week, every two weeks, or every three weeks. In some embodiments, dosages are administered about once or twice every month, every two months, every three months, every four months, every five months, or every six months.

It will be understood by those of ordinary skill that a dosing regimen can be any length of time sufficient to treat the disorder in the subject. The term “chronic” means that the length of time of the dosage regimen can be hours, days, weeks, months, or possibly years.

In some embodiments, the regimen includes one or more cycles of a round of therapy followed by a drug holiday (e.g., no drug). The round of the therapy can be, for example, and of the administrations discussed above. Likewise, the drug holiday can be 1, 2, 3, 4, 5, 6, or 7 days: or 1, 2, 3, 4 weeks, or 1, 2, 3, 4, 5, or 6 months.

The dendrimer complexes can be administered in combination with one or more additional therapeutically active agents, which are known to be capable of treating conditions or diseases discussed above.

### D. Controls

The effect of dendrimer complex compositions can be compared to a control. Suitable controls are known in the art and include, for example, untreated cells or an untreated subject. In some embodiments, the control is untreated tissue from the subject that is treated, or from an untreated subject. Preferably the cells or tissue of the control are derived from the same tissue as the treated cells or tissue. In some embodiments, an untreated control subject suffers from, or is at risk from the same disease or condition as the treated subject.

### E. Combination Therapies

The dendrimer complex compositions can be administered alone, or in combination with one or more additional active agent(s), as part of a therapeutic or prophylactic treatment regime. The dendrimer complex compositions can be administered on the same day, or a different day than the second active agent. For example, compositions including dendrimer complex compositions can be administered on the first, second, third, or fourth day, or combinations thereof.

The term “combination” or “combined” is used to refer to either concomitant, simultaneous, or sequential administration of two or more agents. Therefore, the combinations can be administered either concomitantly (e.g., as an admixture), separately but simultaneously (e.g., via separate intravenous lines into the same subject), or sequentially (e.g., one of the compounds or agents is given first followed by the second).

### VI. Kits

The compositions can be packaged in kit. The kit can include a single dose or a plurality of doses of a composition including one or more cannabinoids associated with or conjugated to a dendrimer (e.g., one or more hydroxyl-terminated PAMAM dendrimers or glucose dendrimers as described in the Examples), and instructions for administering the compositions. Specifically, the instructions direct that an effective amount of the dendrimer composition be administered to an individual with a particular disease/disorder as indicated. The composition can be formulated as described above with reference to a particular treatment method and can be packaged in any convenient manner.

The present invention will be further understood by reference to the following non-limiting examples.

### Example 1: Synthesis of Hydroxyl-Polyamidoamine (PAMAM-OH) Dendrimer Cannabinoid (CBD) Conjugates

The synthesis of PAMAM-OH-CBD conjugates is achieved using a variety of linking chemistries and linkers (both cleavable and non-cleavable). Briefly, the surface hydroxyl groups on PAMAM-OH are modified with a linker to bring a complementary group on the surface that can further react with the complimentary group on the drug linker. Also, the drug of interest is modified with the linker to enable a reaction between a complimentary functional group with a dendrimer-linker.

The linker on the drug is attached via cleavable or non-cleavable linkages. Non-limiting examples of cleavable linkages include esterase sensitive ester bond, glutathione sensitive disulfide bond, phosphatase-sensitive phosphodiester bond, triglycyl peptide linker (CX) capable of lysosomal release, acid cleavable hydrazine linker, and so on. Non-limiting examples of non-cleavable linkages include ether or amide bonds. Amino acids, peptides, polyethylene glycol (n=2-15), and hydrocarbon chains are non-limiting examples of suitable linkers.

The dendrimer-CBD conjugate are designed to (1) provide site specific-targeting by directing the drugs to activated microglia/macrophages expressing the CB2 receptors, thereby decreasing dosage amounts, enhancing efficacy, and reducing side effects related to free drugs, (2) improve water solubility of cannabinoids compared to free drugs by 10-100 fold; (3) circumvent the systemic and dose-related side effects of free CBD drugs via sustained intracellular release at the target site; and (4) reduce or eliminate the unwanted psychotropic effects associated with CB1 agonists by confining dendrimer-CB1 agonist conjugates to peripheral circulation using higher generation dendrimers.

Non-limiting examples of dendrimer-cannabinoid conjugates where CBDs and CBD derivatives are conjugated on the dendrimer surface using various linkers and linking chemistries are described in the following examples.

### Example 2: Synthesis of Dendrimer-Cannabidiol Conjugates Synthesis of Dendrimer-Cannabidiol Conjugate Via Click Chemistry with Enzyme Sensitive Ester Linkage

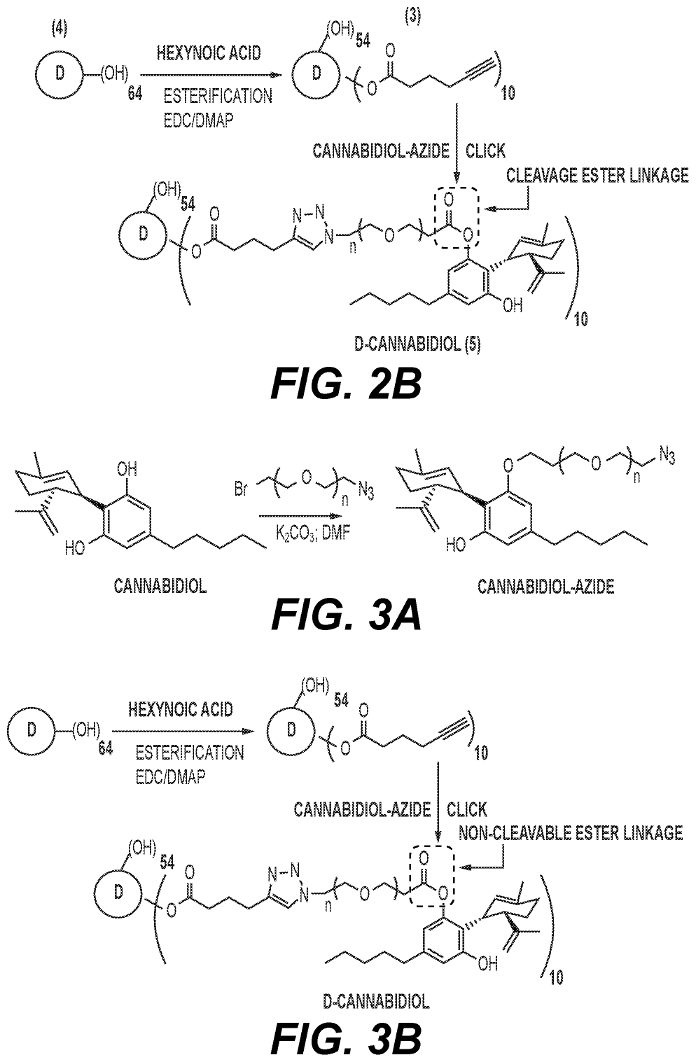

The synthesis of a dendrimer-cannabidiol is achieved using highly efficient copper (I) catalyzed alkyne-azide click (CuAAC) chemistry. The synthesis begins with the modification of Cannabidiol to attach an orthogonal linker with azide terminal through cleavable ester bond (FIGS. 2A and 2B). The purpose of the azide group is to participate in CuAAC reaction with the alkyne functions on the surface of the dendrimer. The hydroxyl group in cannabidiol (1) may be reacted with N3-(OCH2CH₂)ₙ—COOH (2) in the presence of N-(3-dimethylaminopropyl)-N′-ethylcarbodiimide hydrochloride (EDC) and 4-(dimethylamino)pyridine (DMAP), as coupling agents. The crude product is purified using column chromatography to obtain Cannabidiol-azide (FIG. 2A).

On the other hand, the dendrimer can be modified to attach a linker bearing complimentary alkyne groups (FIG. 2B). The hydroxyl-terminated PAMAM dendrimer (D-OH; 4) is reacted with hexynoic acid in the presence of the coupling agents EDC and DMAP to obtain partially alkyne-terminated dendrimer (5) with ten (10) linkers attached (FIG. 2B). Finally, the CuAAC click reaction is performed between dendrimer (5) and Cannabidiol-azide (3) to obtain D-Cannabidiol conjugate with 10 drug molecules attached. The structure confirmation and the purity of all the intermediates and the final conjugate are achieved by the 1^{H} NMR and HPLC respectively.

Synthesis of Dendrimer-Cannabidiol Conjugate Via Click Chemistry with a Non-Cleavable Ester Linkage.

The dendrimer-cannabidiol may also be obtained by click chemistry with a non-cleavable ester linker (FIGS. 3A and 3B). The synthesis begins with the modification of Cannabidiol to attach an orthogonal linker with azide terminal through a non-cleavable ester bond (FIG. 3A). The hydroxyl group in cannabidiol (6) may be reacted with N3-(OCH2CH₂)ₙ—Br (7) in the presence of potassium carbonate (K₂CO₃) and dimethylformamide (DMF), as coupling agents. The product is purified using column chromatography to obtain Cannabidiol-azide (8) as shown in FIG. 3A.

The dendrimer is also modified to attach a linker bearing complimentary alkyne groups (FIG. 3B). The hydroxyl-terminated PAMAM dendrimer (D-OH; 9) is reacted with hexynoic acid in the presence of the coupling agents EDC and DMAP to obtain partially alkyne-terminated dendrimer (10) with ten (10) linkers attached (FIG. 3B). Finally, the CuAAC click reaction is performed between dendrimer (9) and Cannabidiol-azide (3) to obtain D-Cannabidiol conjugate with 10 drug molecules attached. The final conjugate is purified. The structure confirmation and the purity of all the intermediates and the final conjugate are achieved by the 1^{H} NMR and HPLC respectively.

### Example 3: Synthesis of Dendrimer-HU-308 Conjugates

Synthesis of Dendrimer-HU-308 Conjugates Via an Amidation Reaction with Phosphatase-Cleavable Phosphodiester Linkage

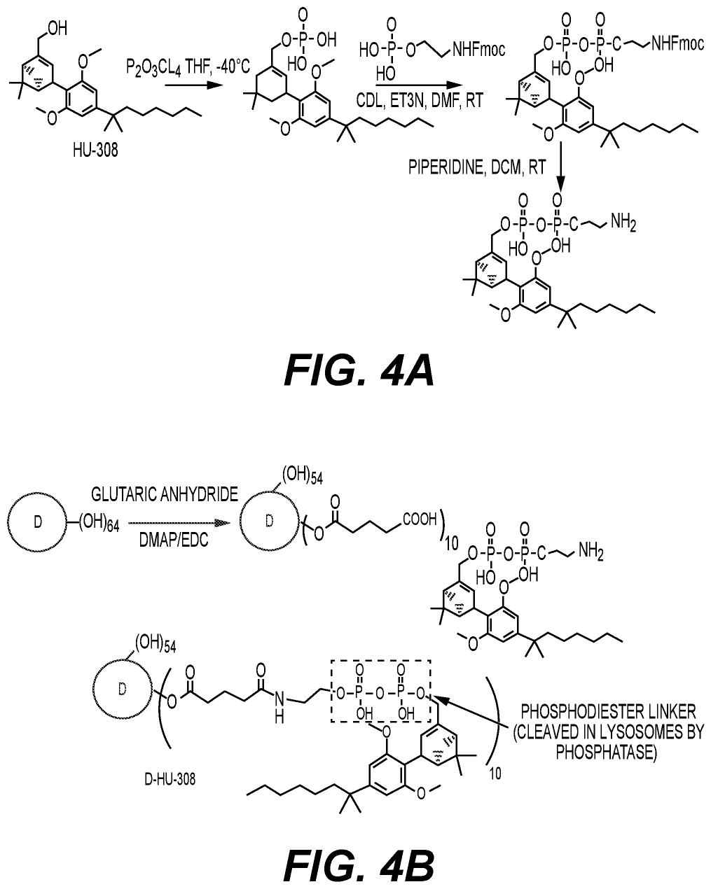

A dendrimer-HU-308 conjugate may be achieved via an amidation reaction with a phosphatase-cleavable phosphodiester linker (FIGS. 4A and 4B). The synthesis begins with the modification of HU-308 to attach a phosphatediester linker. As shown in FIG. 4A, the hydroxyl group in HU-308 (1) is reacted with diphosphoryl chloride (P₂O₃Cl₄) and tetrahydrofuran (THF) at −40° C. to produce compound 1, which is then reacted with FmocHN-CH2CH2-H2PO4 in the presence of triethylamine (Et3N), 1,1′-Carbonyldiimidazole (CDI), and DMF at room temperature to produce compound 2. Then, compound 2 is reacted with DCM and piperidine at room temperature to obtain compound 3 (FIG. 4A).

As shown in FIG. 4B, D-OH is reacted with glutaric anhydride in DMF and DMAP to produce compound 4, which is then reacted with compound 3 to produce the D-HU-308 conjugate containing the phosphatase-cleavable phosphodiester linker.

Synthesis of Dendrimer-HU-308 Conjugate Via Click Reaction with Ester Linkage

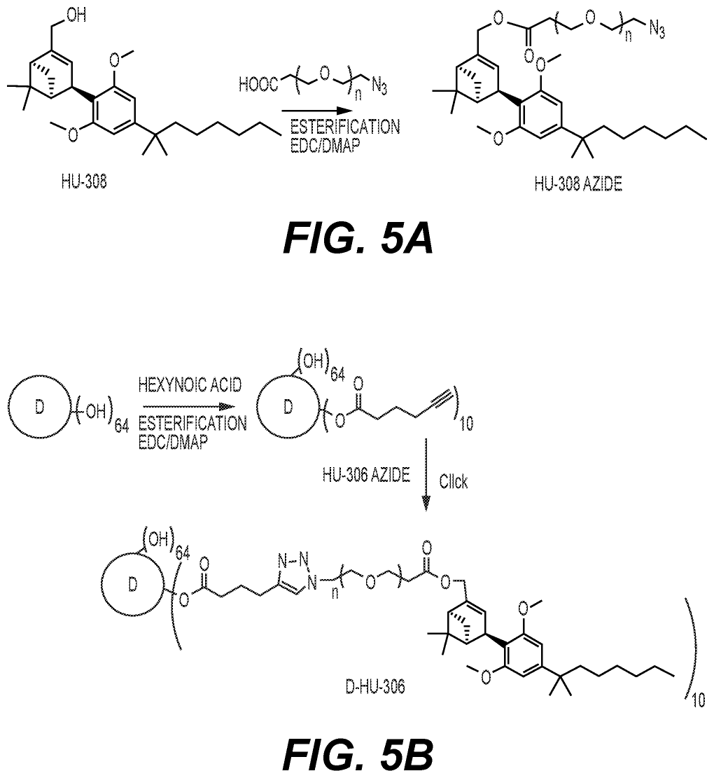

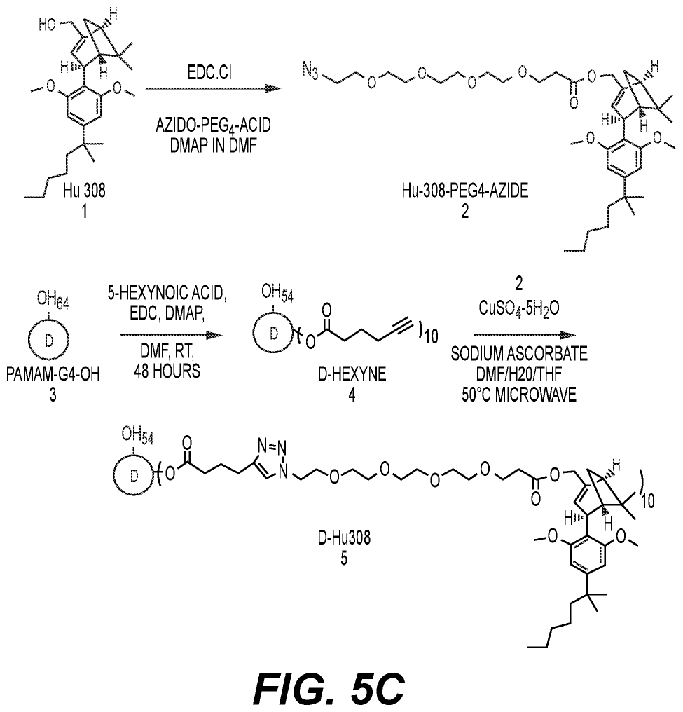

The synthesis of dendrimer-HU-308 may also be achieved by modifying HU-308 to attach an orthogonal linker with azide terminal through a cleavable ester bond (FIGS. 5A and 5B). The hydroxyl group in HU-308 may be reacted with N3-(OCH2CH2)ₙ—COOH in the presence of EDC and DMAP, as coupling agents to obtain HU-308-azide (FIG. 5A). As shown in FIG. 5B, D-OH is reacted with hexynoic acid in the presence of the coupling agents EDC and DMAP to obtain partially alkyne-terminated dendrimer with ten (10) linkers attached (FIG. 5B). Finally, the click reaction is performed between dendrimer and HU-308-azide to obtain D-HU-308 conjugate with 10 drug molecules attached. The structure confirmation and the purity of all the intermediates and the final conjugate are achieved by the 1^{H} NMR and HPLC respectively. FIG. 5C is a schematic of the conjugation of the HU-308 to a PAMAM-G4-OH dendrimer.

Synthesis of Dendrimer-HU-308 Conjugate Via Click Reaction with Glutathione Sensitive Disulfide Linkage

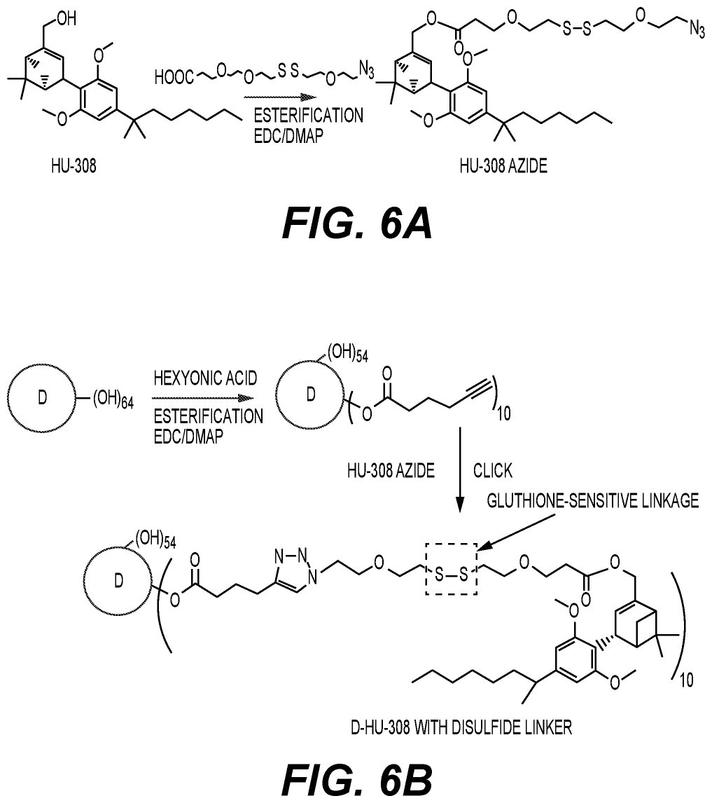

The dendrimer-HU-308 may also be synthesized via a click reaction with a glutathione sensitive disulfide linker (FIGS. 6A and 6B). The hydroxyl group in HU-308 may be reacted with N3-SS-PEG2-acid in the presence of EDC and DMAP, as coupling agents to obtain HU-308-azide (FIG. 6A).

On the other hand, D-OH is reacted with hexynoic acid in the presence of the coupling agents EDC and DMAP to obtain partially alkyne-terminated dendrimer with ten (10) linkers attached (FIG. 6B). Finally, the click reaction is performed between the dendrimer and HU-308-azide to obtain D-HU-308 conjugate with 10 drug molecules attached (FIG. 6B). The structure confirmation and the purity of all the intermediates and the final conjugate are achieved by the 1^{H} NMR and HPLC respectively.

### Example 4: Synthesis of Dendrimer-Tetrahydrocannabinol Conjugates

Synthesis of Dendrimer-Tetrahydrocannabinol Conjugate Via Click Reaction with Esterase Sensitive Ester Linkage.

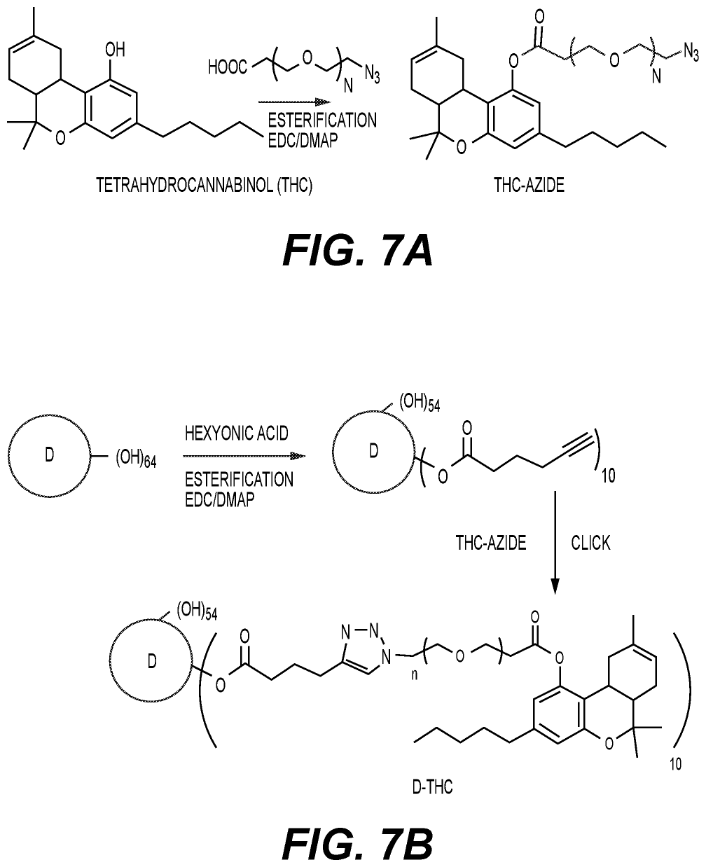

The synthesis of a dendrimer-tetrahydrocannabinol (D-THC) conjugate may be achieved via click reaction with a esterase sensitive ester linker (FIGS. 7A and 7B). As shown in FIG. 7A, the hydroxyl group in THC may be reacted with N3-(OCH2CH2)ₙ—COOH in the presence of EDC and DMAP, as coupling agents to obtain THC-azide. Then, the D-OH is reacted with hexynoic acid in the presence of the coupling agents EDC and DMAP to obtain partially alkyne-terminated dendrimer with ten (10) linkers attached (FIG. 7B). Finally, the click reaction is performed between the dendrimer and THC-azide to obtain D-THC conjugate with 10 drug molecules attached. The structure confirmation and the purity of all the intermediates and the final conjugate are achieved by the 1H NMR and HPLC respectively.

Synthesis of Dendrimer-Tetrahydrocannabinol Conjugate Via Click Reaction with Triglycyl Peptide Linkage for Lysosomal Release.

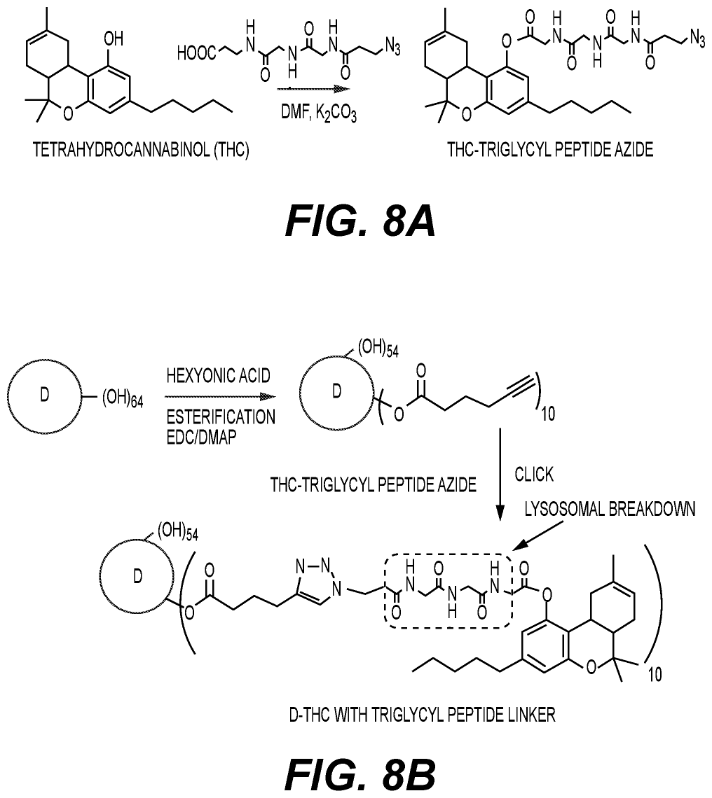

The synthesis of dendrimer-tetrahydrocannabinol (D-THC) conjugate may also be achieved via click reaction with a triglycyl peptide linker (FIGS. 8A and 8B). The triglycyl peptide linker permits lysosomal mediated cleavage of the dendrimer-THC conjugate. The hydroxyl group in THC may be reacted with triglycyl peptide azide in the presence of DMF and K₂CO₃, as coupling agents to obtain THC-triglycyl peptide azide (FIG. 8A). As shown in FIG. 8B, D-OH is reacted with hexynoic acid in the presence of the coupling agents EDC and DMAP to obtain partially alkyne-terminated dendrimer with ten (10) linkers attached. Finally, the click reaction is performed between the dendrimer and THC-triglycyl peptide azide to obtain D-THC conjugate with a triglycyl peptide linker and 10 drug molecules attached. The structure confirmation and the purity of all the intermediates and the final conjugate are achieved by the 1H NMR and HPLC respectively.

### Example 5: Synthesis of Dendrimer-Anandamide Conjugates

Synthesis of Dendrimer-Anandamide Conjugate Via Click Reaction with Esterase Sensitive Ester Linkage.

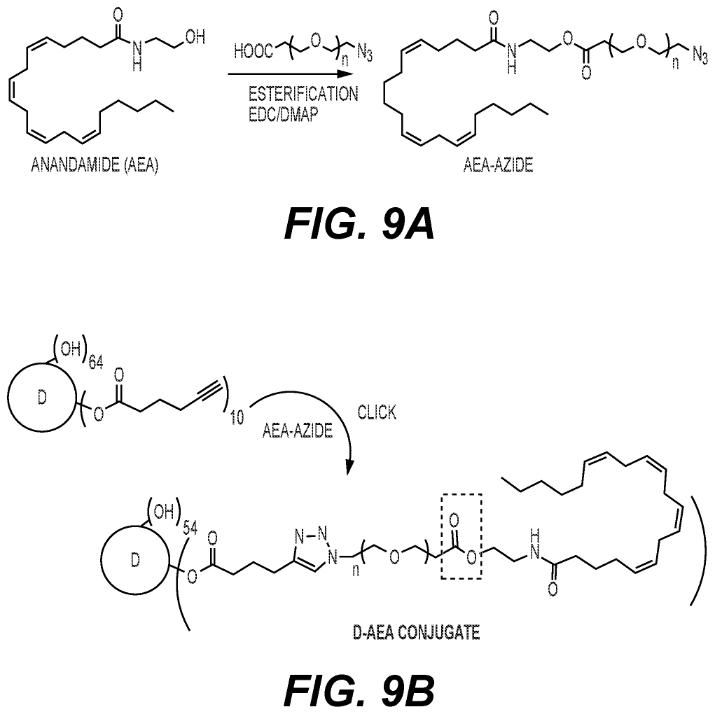

A dendrimer-anandamide (D-AEA) conjugate may be synthesized via a click reaction with an esterase sensitive ester linker (FIGS. 9A and 9B). The hydroxyl group in AEA may be reacted with N3-(OCH2CH₂)ₙ—COOH in the presence of EDC and DMAP, as coupling agents to obtain AEA-azide (FIG. 9A). AEA-azide is then conjugated to the dendrimer (D-OH) via click reaction to obtain the D-AEA conjugate as shown in FIG. 9B.

Synthesis of Dendrimer-Anandamide Conjugate with Non-Cleavable Ether Linkage.

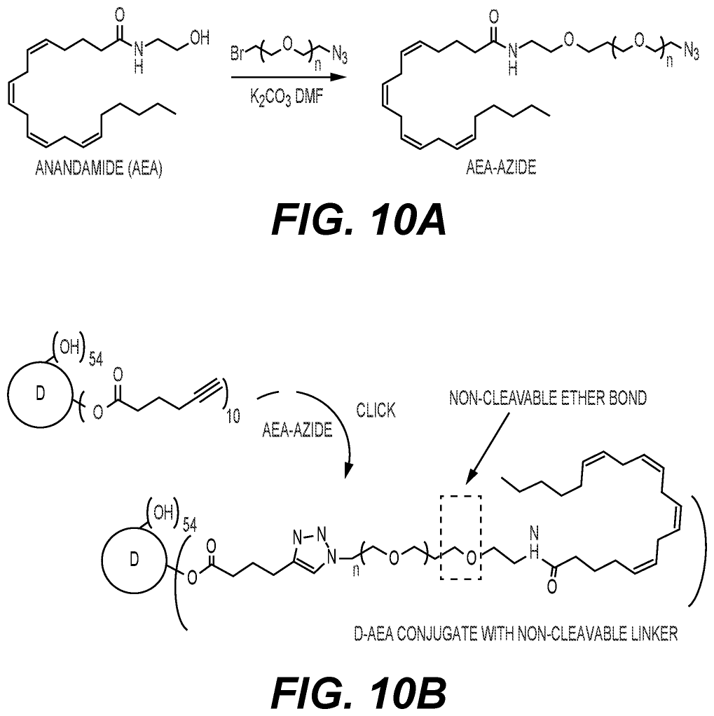

The synthesis of a dendrimer-anandamide (D-AEA) conjugate may also be achieved via click reaction with a non-cleavable ether linker (FIGS. 10A and 10B). The hydroxyl group in AEA may be reacted with N3-(OCH2CH2)ₙ-Br in the presence of K₂CO₃ and DMF, as coupling agents to obtain AEA-azide (FIG. 10A). AEA-azide is conjugated to the dendrimer (D-OH) via click reaction to obtain the D-AEA conjugate with a non-cleavable linker as shown in FIG. 10B.

### Example 6: Synthesis of Dendrimer-2-Arachidonoylglycerol (D-2-AG) Conjugate Via Click Reaction with Esterase Sensitive Ester Linkage

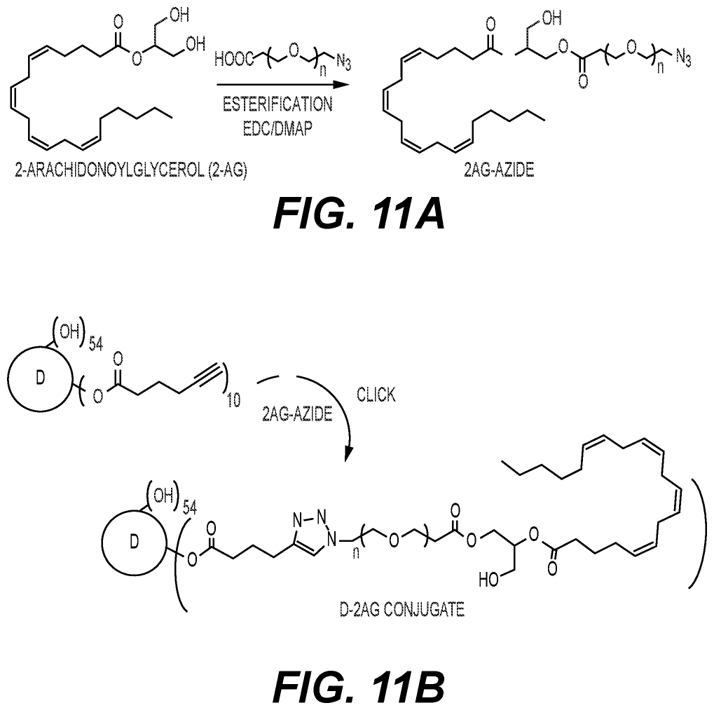

A dendrimer-2-arachidonoylglycerol (D-2-AG) conjugate may be synthesized via click reaction with a non-cleavable an esterase sensitive ester linkage (FIGS. 11A and 11B). The hydroxyl group in 2-AG may be reacted with N3-(OCH2CH2)ₙ—COOH in the presence of EDC and DMAP, as coupling agents to obtain 2-AG-azide (FIG. 11A). 2-AG-azide is conjugated to the dendrimer (D-OH) via click reaction to obtain the D-2-AG conjugate with an esterase sensitive ester linker as shown in FIG. 11B.

### Example 7: Synthesis and Binding of Cannabinoid Drugs with Hydroxyl-Terminated PAMAM, Glucose Dendrimers, and Hydroxylated Bis MPA Dendrimers

This methodology is targeted to specific receptors on cells such as neurons, microglia, macrophages and other cells. These receptors can be anywhere in the body. For example, CB1/CB2 receptors are present throughout the body in different organs, including the brain. Conjugates as described herein bind to these receptors. Therefore, the ‘intrinsic cellular targeting’ of these dendrimers, for example, targeting of hydroxyl dendrimer to microglia and/or macrophages, and targeting of glucose dendrimers to neurons, are secondary to the binding to and action on the specific receptors.

Targeting of specific receptors can be used to achieve desired results. In one embodiment the conjugates are targeted to CB1 and can cause agonism, antagonism, reverse or inverse agonism. In another embodiment, conjugates are targeted to CB2 and can cause agonism, antagonism, or reverse agonism, or a combination of activity against CB1/CB2, TRPV1, and other receptors.

When combined with the cellular targeting capability of glucose dendrimers (injured neurons (primary), microglia/macrophages (secondary), or hydroxyl dendrimers (microglia/macrophages), the conjugates can be targeted more specifically to the specific cannabinoid receptors on specific cells in specific organs.

The dendrimer conjugates not only assist in targeting but improve water solubility more than 200-fold as compared to the drug not bound to the dendrimer. This greatly facilitates formulation and delivery.

The following example using tryptamine (as a non-limiting example) demonstrates efficacy of the conjugates for selective targeting, alteration of binding affinity, and Hu-308 for water solubility.

### Material and Methods

Unless stated otherwise, reactions were performed in flame dried glassware under a positive pressure of nitrogen using dry solvents.

Commercial grade reagents and anhydrous solvents were purchased from chemical suppliers and used without further purification. 1-Ethyl-3-(3-dimethylaminopropyl) carbodiimide (EDC.HCl), N, N-diisopropylethylamine (DIPEA), 4-(dimethylamino)pyridine (DMAP) trifluoracetic acid (TFA), anhydrous dichloromethane (DCM), N,N′-dimethylformamide (DMF) were purchased from Sigma-Aldrich (St. Louis, MO, USA). Cyanine 5 (Cy5)-mono-NHS ester was purchased from Amersham Bioscience-GE Healthcare. Deuterated solvents dimethylsulfoxide (DMSO-d6), water (D₂O), and Chloroform (CDCl₃) were purchased from Cambridge Isotope Laboratories Inc. (Andover, MA). Ethylenediamine-core polyamidoamine (PAMAM) dendrimer, generation 4.0, hydroxy surface (G4-OH; diagnostic grade; consisting of 64 hydroxyl end-groups), methanol solution (13.75% w/w) was purchased from Dendritech Inc. (Midland, MI, USA). Dialysis membranes were purchased from Spectrum Laboratories Inc. (Rancho Dominguez, CA, USA). Hu308, Tryptamine, 1-(2-amino-1-(4-methoxyphenyl)ethyl)cyclohexanol, Nor-ketamine, 5-hydroxy tryptamine, psilocybin analog, psilocyn analog and cannabidiol drugs were purchased from Cayman Chemicals.

### Synthesis of Hydroxyl-PAMAM Dendrimer Drug Conjugates.

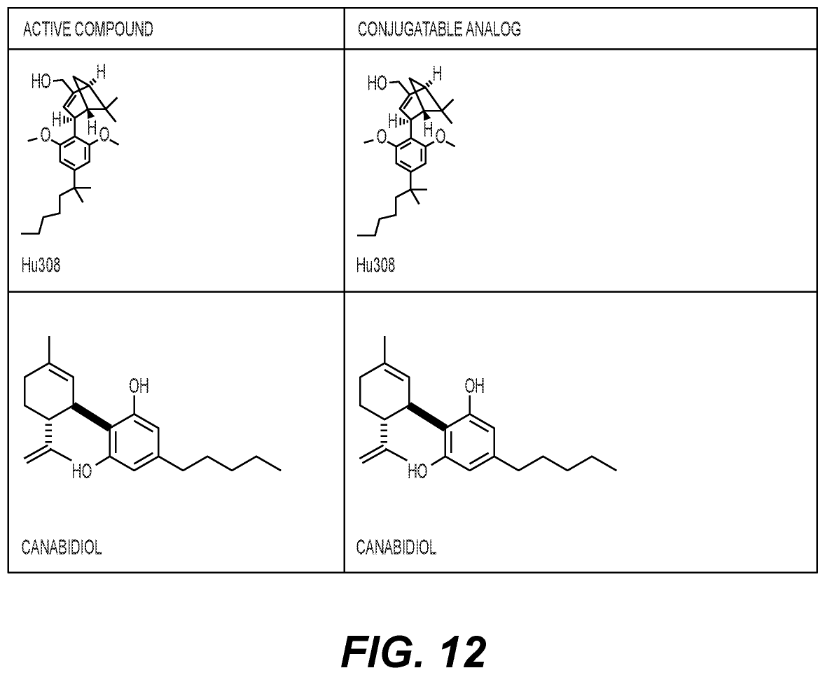

The PAMAM-G4-OH (D4-OH) dendrimer composed of about 64 terminal hydroxyl groups was used for the synthesis. After each synthetic step, the product was purified via dialysis in DMF for 24 h to eliminate small molecule impurities followed by water dialysis to remove DMF. ¹H NMR (in DMSO-d6 and D20) and analytical HPLC were used to confirm the intermediates and final product formation and purity. The mono-functional D4-OH was functionalized with alkyne group by treatment of 5-hexynoic acid under standard esterification conditions using EDC.HCl and 4-DMAP in DMF for 36 h at room temperature to yield the D-hexyne bifunctional dendrimer. The number of alkyne groups on dendrimer surface was chosen to be kept at ˜10-15 to maintain the overall water solubility of the conjugate. The crude product was dialyzed by 1 kDa membrane against ultrapure water for 24 h to remove low molecular weight impurities via selective diffusion across the semi-permeable dialysis membrane. The ¹H NMR and analytical HPLC were used to confirm the product formation and purity of the intermediates and final products. These are shown in FIG. 12A-12C.

### Synthesis of D-Hexyne

A solution of PAMAM G4-OH 1 (10.00 g, 0.7 mmol) in DMF (50 mL) was treated with 5-Hexynoic acid (1.40 g, 12.6 mmol), DMAP (2.41 g, 12.6 mmol) and stirred at room temperature for 5 min. Then EDC.HCl (1.54 g, 12.6 mmol) was added in portions to the reaction mixture over the period of 5 min. The reaction mixture was stirred at room temperature for 36 h. The crude product was transferred to 1 kD MW cut-off cellulose dialysis tubing and dialyzed against DMF 12 h followed by water for 24 h. The aqueous layer was frozen and lyophilized to yield D-hexyne as a hygroscopic white solid (75% yield). ¹H NMR (500 MHz, DMSO-d6) 8.21-7.57 (m, internal amide H), 4.71 (s, GABA amide H, 50H), 4.01 (t, 22-24 CH₂), 3.5-2.1 (m, dendrimer CH₂) 1.71-1.59 (m, 22-24 CH₂). HPLC C18 retention time 4 min: purity ˜99%.

### Synthesis and Purification of Hu308-Azide

This method can be used for other cannabinoids. A solution of Hu308 in anhydrous dimethylformamide (DMF) was treated with hexanoic acid-PEG4-Azide in anhydrous DMF, DMAP and EDC. The reaction was stirred under N2 at room temperature for 24 h. The reaction was monitored using thin-layer chromatography and upon completion crude reaction mixture was concentrated, taken up in saturated sodium bicarbonate and extracted with DCM, then the organic layer was subsequently washed with saturated sodium bicarbonate solution, followed by saturated ammonium chloride solution and finally with brine. The organic layer was then dried with anhydrous sodium sulfate, filtered, and concentrated in vacuo. The crude product was dried under rotovap and purified with a CombiFlash chromatography over silica gel 40% ethyl acetate/hexane gradient as eluent. The desired Hu308-PEG4-azide was obtained as a colorless oil.

### Synthesis of Dendrimer-Drug Conjugate.

The solution of D-Hexyne and drug-azide in DMF (5 mL) was treated with copper sulfate pentahydrate (CuSO₄·5H₂O) and sodium ascorbate in water. The reaction mixture was stirred and heated for 10 h at 50° C. in a microwave synthesizer. On completion, the reaction mixture was dialyzed against DMF in 1 KDa cut-off cellulose dialysis tubing. To this solution, EDTA (50 μL, 0.5M) solution was added for copper removal by chelation. The DMF dialysis was followed by water dialysis overnight. The drug loading is calculated by proton integration where peaks corresponding to dendrimer and drug are compared.

TABLE 2
Physical properties of D-drug conjugates.

| | | Water | | |
|---|---|---|---|---|
| | | Solubility of | | |
| | | dendrimer- | | |
| | | drug | | |
| Dendrimer | Water | conjugates | | |
| drug | Solubility | (based on | Number of | |
| conjugate | Free drug* | free drug) | attachments | DLS/nm |
| D-Hu308 | <0.1 mg/ml | 20 mg/mL | ~10-11 | 4.9 ± 0.2 |

### G2-Glucose Dendrimer (GD2)—Drug Conjugate

Generation 2 glucose dendrimer (GD2) consists of 24 glucose molecules (96 surface hydroxyl groups) used for conjugation. Glucose dendrimers primarily are made of glucose moieties comprised of the central core of Di-pentaerythritol and one or more branching units of monosaccharide glucose molecules. Unlike hydroxyl-terminated PAMAM dendrimer, glucose dendrimers primarily are taken up by injured neurons and found to specifically target hyperexcitable neurons in both culture and in vivo mouse model.

### Synthesis and Characterization of Glucose Dendrimer(GD2).

The GD synthesis was begun by reacting hexapropargylated core with AB4, β-D-glucose-PEG4-azide building via click reaction to obtain generation 1 glucose dendrimer (GD1). The OH groups on GD1 were propargylated to obtain GD1-Acetylene24, which was reacted with β-D-glucose-PEG4-azide to obtain generation 2 (GD2) with 24 glucose moieties, providing 96 surface hydroxyl groups. Further the Cy5 fluorescent tag was attached on GD2 by propargylation of ˜2-3 hydroxyl groups to bring alkyne containing GD2 dendrimer. The GD intermediates and final products were purified using dialysis and characterized using ¹H NMR. The physicochemical properties of GD2 dendrimer were also evaluated (Table 3).

TABLE 3
Physiochemical Properties of GD2

| Size | 4.4 | nm |
|---|---|---|
| Zeta Potential | −6.0 | |
| Number of Glucose | 24 | |
| HPLC purity | >99% | |
| Molecular weight | 12800 | Da |

### Synthesis and Characterization of GD2-Drug Conjugates

Hu308 drugs were conjugated to the GD2-Hexynoic acid dendrimer using click chemistry strategy. The linker attached drug moieties were conjugated to glucose dendrimer using Cu(I) catalyzed click (CuAAC) reaction in the presence of catalytic amount of CuSO₄·5H₂O and sodium ascorbate to obtain GD2-drug conjugate. The traces of copper were removed by dialyzing with ethylenediaminetetraacetic acid (EDTA). The final GD2-drug conjugates were characterized by NMR and HPLC.

TABLE 4
Physical properties of GD2-drug conjugates

| | | Water Solubility | |
|---|---|---|---|
| | Water | dendrimer-drug | |
| Dendrimer drug | Solubility | conjugates | Number of |
| conjugate | Free drug* | (based on drug) | attachments |
| GD-Hu308 | <0.1 mg/ml | 25 mg/mL | ~10 |

### Results

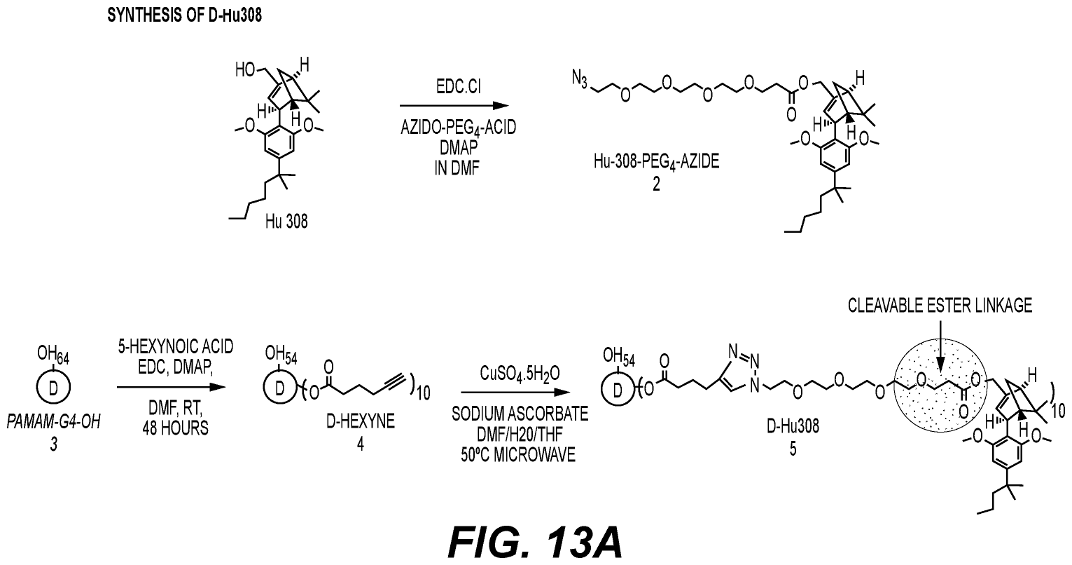

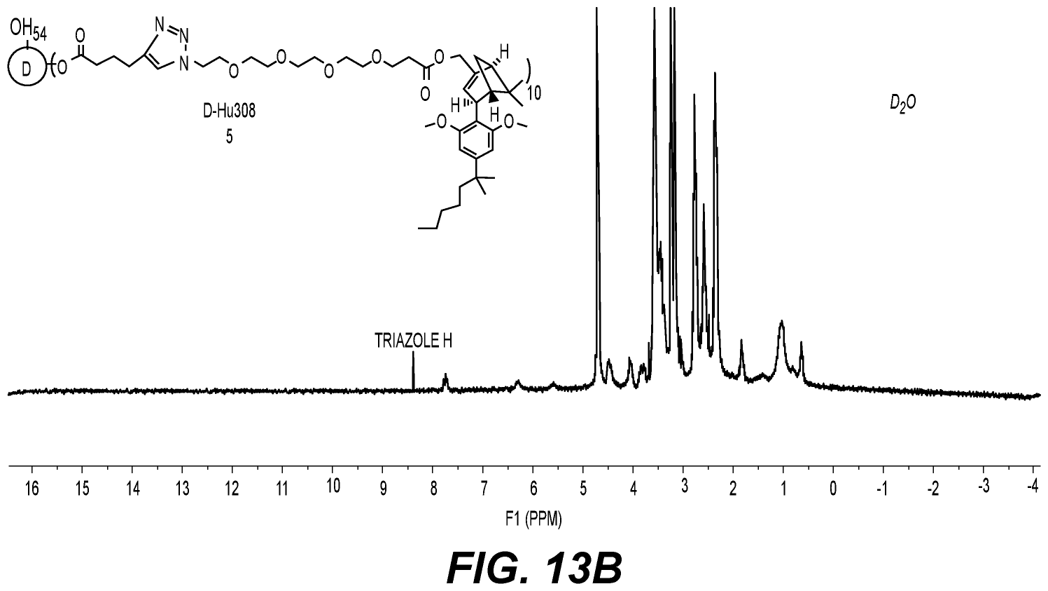

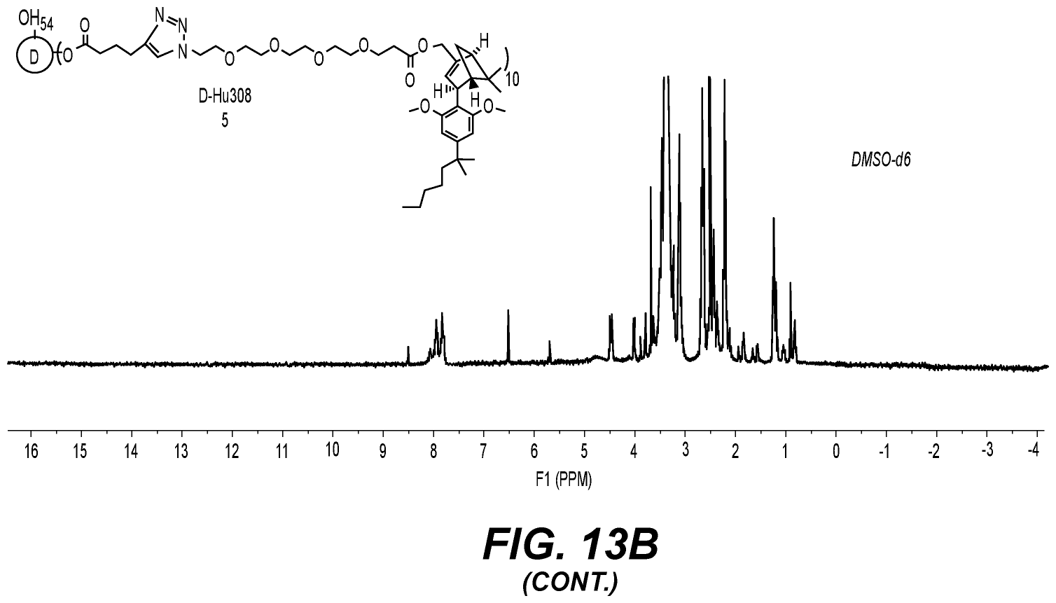

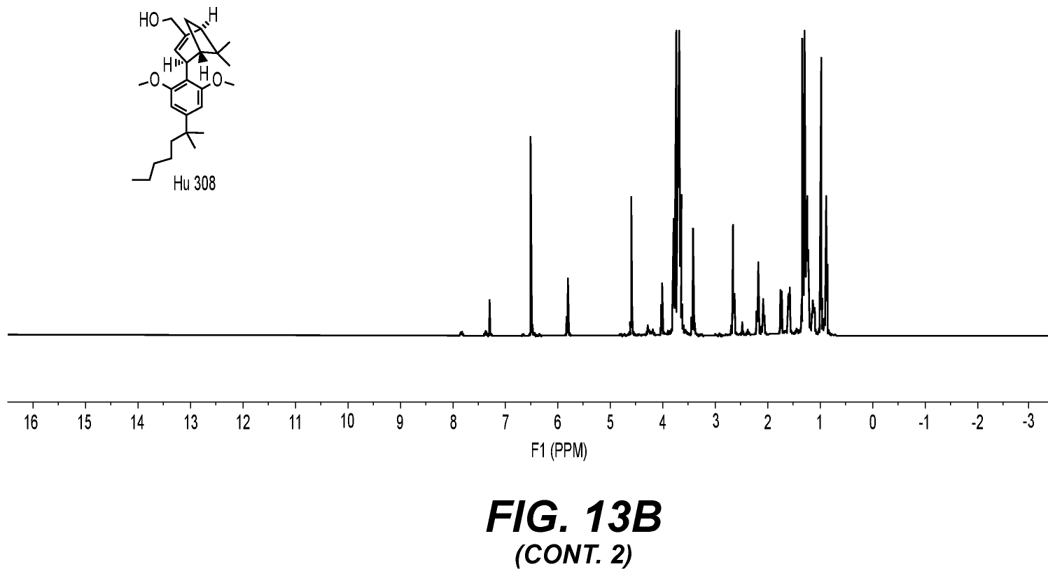

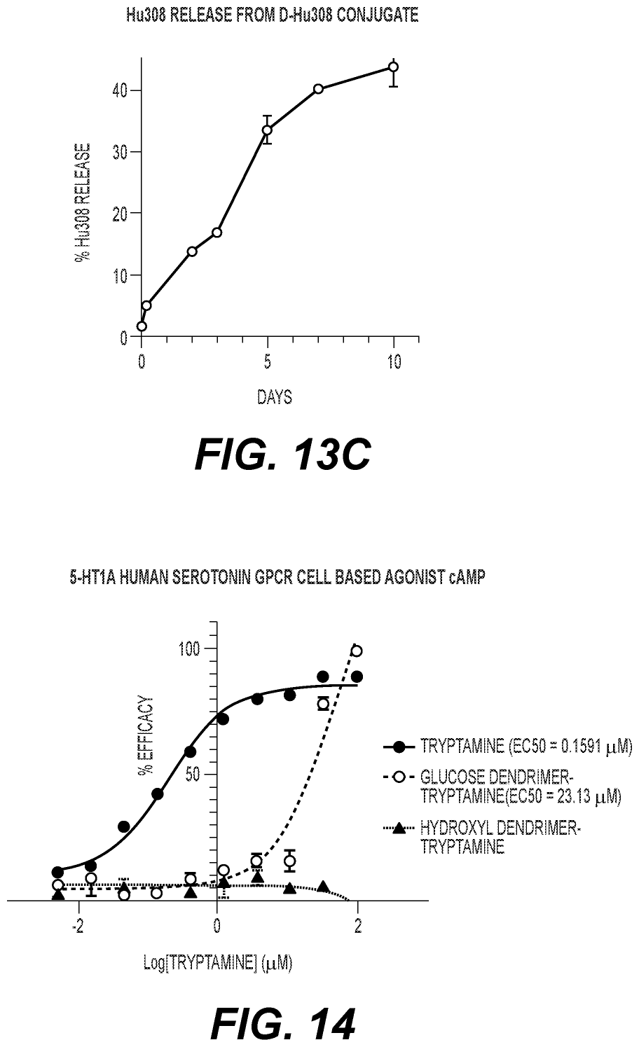

The compounds are shown in FIG. 12. FIG. 13A-13B show the synthesis and structures. FIG. 13C show the binding and release of the compounds.

FIG. 14 is a graph of % efficiency of binding in the 5-HT1A human serotonin GPCR cell based agonist cAMP assay versus log of tryptamine in μM.

Both hydroxyl and GD dendrimers have OH surface groups. When tryptamine was conjugated to these dendrimers with the same linking chemistry, very different affinities were seen to serotonin and other receptors. FIG. 13C. Binding assays showed that the conjugates had less affinity than free drug in cell-based binding assays (indicative of in vivo efficacies). The lower affinity may enable less tight binding and may enable modulation of the undesirably strong effects of the drugs on this receptor. Second, the hydroxyl dendrimer conjugate was not active, but the glucose dendrimer conjugate was active. This is not expected and may be due to the differential internal structure of the glucose and hydroxyl dendrimers. The drug may fold into the hydrophobic core of the hydroxyl dendrimer but may open outwards in the hydrophilic interior of the glucose dendrimer.

The conjugates can be active with or without releasing the drug, and can be engineering to release or not release the drug using appropriate linking chemistry described here. Binding affinities measure the activity of the intact conjugates. For example, Hu-308 conjugates release the drugs over a period of time (FIG. 13C) and are active both in the conjugated and the released forms. The cannabidiol (CBD) conjugates can be engineered to not release the drug and be active in the conjugated form.

### Example 8. Localization of Dendrimer-Hu308 in Peripheral Nerve Injury/Sciatic Nerve Injury

### Materials and Methods

Animals that underwent sciatic nerve ligation were administered by intravenous injection of dendrimer-Hu308 tagged with the fluorescent dye Cy5 after ten days. These were perfused and imaged after 24 hours. Microglia on the injured side are known to overexpress the CB2 receptor that modulates neuropathic pain.

### Results

In a rodent model of chronic neuropathic pain induced by injury to the sciatic nerve, dendrimer conjugated to the CB2 receptor agonist Hu308 binds CB2 receptors that are overexpressed in microglia around the site of injury. Hu308 binds the intracellular portion of the CB2R and the dendrimer-Hu308 is seen to localize at a high concentration inside the glia, enabling intracellular binding of the CB2 receptor.

Hydroxyl-terminated dendrimer-Hu308-Cy5 localizes in microglia only around the area of sciatic nerve injury. These microglia are known to overexpress CB2R after injury. Other the other hand, glucose-dendrimer GD tagged with Cy5 localized in injured neurons along the nerve and in neurons and microglia in the dorsal root ganglion on the same side as the injury in the acute period immediately after injury. GD2-Cy5 localized in neurons near the ligation site and in neurons and glia in dorsal root ganglion. This indicates that GD-Hu308 can bind the CB2 receptors on injured neurons and glia immediately after peripheral nerve injury.

Modifications and variations of the present invention will be apparent to those skilled in the art and are intended to come within the scope of the appended claims.

1. A composition comprising dendrimers, preferably hydroxyl-terminated dendrimers, sugar-terminated dendrimers, and/or sugar-based dendrimers, conjugated to at least one natural cannabis derivative or synthetic cannabinoid compound, optionally where the conjugation is covalent.

2. The compositions of claim 1, where the compound is covalently conjugated by a covalent link between a modified or unmodified surface group or interior groups of the dendrimer, including an ester, amide, disulfide, or ether linkage.

3. The composition of claim 1, wherein the covalent link between the dendrimer and cannabinoid compound is cleaved following administration.

4. The composition of claim 1 where the dendrimer is a polyamidoamine (PAMAM) dendrimer of generation 1-generation 7 (such as generation 1, generation 2, generation 3, generation 4, generation 5, generation 6, or generation 7), preferably with between greater than 40 and 100% of the surface groups being hydroxylated.

5. The composition of claim 1 wherein the dendrimer is a generation 1-generation 7 PAMAM dendrimer (such as generation 1, generation 2, generation 3, generation 4, generation 5, generation 6, or generation 7), optionally with end groups selected from the group consisting of COOH, NH₂, and acetamide.

6. The composition of claim 1 where the dendrimer is a generation 1-Generation 7 PAMAM dendrimer (such as generation 1, generation 2, generation 3, generation 4, generation 5, generation 6, or generation 7), modified by a sugar moiety, wherein the sugar group is selected from the group consisting of glucose, galactose, mannose, and fructose.

7. The composition of claim 1 where the dendrimer is a dendritic polymer, optionally a hyperbranched polymer.

8. The composition of claim 1 where the dendrimer is a PAMAM dendrimer (such as a hydroxyl-terminated PAMAM dendrimer) of generation 1-generation 7, preferably with greater than 50% of the functional groups in the form of hydroxyl (OH) groups.

9. The compositions of claim 1 where the dendrimer is a glucose dendrimer preferably generation 1, generation 2, or generation 3 glucose dendrimers, preferably made of glucose and optionally ethylene glycol building blocks, with >10 surface glucose moieties.

10. The compositions of claim 1 where the dendrimer is a galactose dendrimer, made of galactose and optionally ethylene glycol building blocks, with >10 surface sugar moieties.

11. The compositions of claim 1 where the dendrimer is a sugar dendrimer, made of sugar and optionally ethylene glycol building blocks, with >10 surface sugar moieties, optionally where the sugar building blocks are mannose or galactose.

12. The composition of claim 1 where the cannabinoid is cannabidiol (CBD) or a CBD-analog.

13. The composition of claim 1 where the cannabinoid is tetrahydrocannabinol (Δ9-THC) or an analog thereof.

14. The composition of claim 1 where the cannabinoid is a CB1 agonist, preferably selected from the group consisting of arachidonylcyclopropylamide (ACPA) and methanandamide.

15. The composition of claim 1 where the cannabinoid is a CB1 antagonist, preferably selected from the group consisting of rimonabant, AM251, and AM281.

16. The composition of claim 1 where the cannabinoid is a CB2 agonist, preferably wherein the CB2 agonist is selected from the group consisting of HU-308, JWH-133, AM1241, GW405833, and GW842166X.

17. The composition claim 1 where the cannabinoid is a CB1 and/or CB2 agonist, wherein the cannabinoid is selected from the group consisting of anandamide, Δ9-THC, and 2-Arachidonoyl glycerol.

18. The composition of claim 1 where the cannabinoid is a TRPV1 agonist, preferably CBD.

19. The composition of claim 1 where the drug is a CB1 antagonist or CB2 agonist, preferably tetrahydrocannabivarin (THCV) and THCV-analogs.

20. The composition of claim 1 wherein the cannabinoid is a CB1 antagonist, or a CB2 antagonist.

21. The composition of claim 1, wherein the dendrimer-cannabinoid compositions have at least a 10-fold increase in drug solubility in comparison to the free cannabinoid.

22. The composition of claim 1 where the dendrimer-cannabinoid conjugates further comprise pharmaceutically acceptable excipients and optionally non-cannabinoid active agents.

23. The composition of claim 1, where the conjugate is confined to the peripheral circulation by the use of higher generation dendrimer, preferably generation 4, 5, or 6 PAMAM dendrimer, generation 2, 3, or higher glucose dendrimer, or functionalized with PEG.

24. The composition of claim 1, wherein the at least one natural cannabis derivative or synthetic cannabinoid compound is conjugated to the dendrimers via a spacer comprising a hydrocarbon (such as an alkylene), a diethylene glycol moiety, oligoethylene glycol chain, a triazole moiety, or a combination thereof, preferably wherein the spacer comprises a triazole moiety.

25. A pharmaceutical composition comprising the composition of claim 1, and a pharmaceutically acceptable carrier or excipient.

26. The pharmaceutical composition of claim 25, in a form selected from hydrogels, nanoparticle, microparticles, suspensions, powders, tablets, capsules, creams, solutions, or a combination thereof.

27. A method of administering the dendrimer-cannabinoid conjugates of claim 1 to an individual in need thereof, preferably administered via a route selected from the group consisting of intranasal, intravenous, oral, subcutaneous, inhalable, intraperitoneal, topical, and intrathecal.

28. The method of claim 27, wherein the individual has an ocular disorder, preferably wherein the dendrimer-cannabinoid conjugates are administered via intravitreal, suprachoroidal, subconjunctival, topical, or systemic administration.

29. The method of claim 27 comprising treating an individual with peripheral nerve pain such as peripheral neuropathy or chemotherapy induced pain.

30. The method of claim 27 comprising treating an individual in need of treatment of nervous disorders, including epilepsy and seizures, where the cannabinoid is cannabidiol (CBD) or a CBD-analog.

31. The method of claim 27 comprising treating an individual in need thereof for obesity-related metabolic disorders, mental illness, liver fibrosis, or nicotine addiction, with a CB1 antagonist, preferably selected from the group consisting of rimonabant, AM251, and AM281.

32. The method of claim 27 comprising treating an individual in need thereof for symptoms of arthritis, diabetic nephropathy, renal interstitial disease, fibrosis, multiple sclerosis, irritable bowel syndrome, autoimmune disorders, traumatic brain injury, acute pain, chronic inflammatory pain, neuropathic pain, ocular pain, or inflammatory disorders where the cannabinoid is a CB2 agonist, preferably wherein the CB2 agonist is selected from the group consisting of HU-308, JWH-133, AM1241, GW405833, and GW842166X.

33. The method of claim 27 comprising treating an individual in need thereof for symptoms of Neuropathic pain, Cancer-related pain, Alzheimer's Disease, Parkinson's Disease, HIV-associated neurological disorders, wherein the conjugate is a dendrimer-CB1/CB2 agonist conjugate, preferably wherein the cannabinoid is selected from the group consisting of anandamide, Δ9-THC, and 2-Arachidonoyl glycerol.

34. The method of claim 27, comprising treating an individual in need of treatment of glaucoma via IOP lowering or neuroprotection, where the cannabinoid is selected from a phytocannabinoid, synthetic cannabinoid, endocannabinoid, and combinations thereof.

35. A method of treating an individual in need thereof for symptoms of depression, addiction, renal disorders (e.g., AKI), diabetic neuropathy, hepatic fibrosis, hepatic encephalopathy or epilepsy comprising administering dendrimer-CB1 antagonist/CB2 agonist conjugates, preferably tetrahydrocannabivarin (THCV) and THCV-analogs.

36. The method of claim 27 comprising targeting of the compositions to neurons, or neurons in the disease-affected regions enabled by conjugation of the CB1 agonists or antagonists to glucose dendrimers which target injured neurons.

37. The method of claim 27 comprising targeting peripheral neurons without appreciable uptake into the brain, by administering through the higher generation and/or surface functionalized dendrimers the cannabinoids.

38. A method of targeting therapeutic, prophylactic or diagnostic agent to neurons in a disease or disorder-affected region of an individual comprising administering the agent with a CB1 agonist or antagonist conjugated to a dendrimer, preferably hydroxyl-terminated dendrimers, sugar-terminated dendrimers, and/or sugar-based dendrimers.

39. The method of claim 38 wherein the dendrimer is a PAMAM dendrimer.

40. A method of targeting therapeutic, prophylactic or diagnostic agent to neurons in a disease or disorder-affected region of an individual comprising administering the agent to reactive microglia/macrophages for intracellular delivery of the agent, wherein the agent is administered with a conjugate of CB2 agonists or antagonists to hydroxyl-terminated PAMAM or glucose dendrimers.

41. The method of claim 38 wherein the agent is selected from the group consisting of non-opioids, non-cannabinoids, or cannabinoids, either conjugated or non-conjugated to the dendrimer.

42. The method of claim 38 administered in combination with radiation therapy, exercise, or other co-treatments.

43. The method of claim 38 wherein the agent is an imaging or therapeutic agent for PET, MRI or radiation.
